(19) United States
(12) Patent Application Publication
(10) Pub. No.: US 2023/0044248 A1
(43) Pub. Date: Feb. 9, 2023

(54) COMBINED INHIBITION OF PD-1, TGFBeta AND ATM TOGETHER WITH RADIOTHERAPY FOR THE TREATMENT OF CANCER

(71) Applicants: ARES TRADING S.A., Aubonne (CH); GLAXOSMITHKLINE INTELLECTUAL PROPERTY (NO. 4) LTD., Brentford Middlesex (GB)

(72) Inventors: Yan LAN, Billerica, MA (US); Adam S. LAZORCHAK, Billerica, MA (US)

(73) Assignees: ARES TRADING S.A., Aubonne (CH); GLAXOSMITHKLINE INTELLECTUAL PROPERTY (NO. 4) LTD., Brentford Middlesex (GB)

(21) Appl. No.: 17/770,420

(22) PCT Filed: Nov. 2, 2020
(86) PCT No.: PCT/EP2020/080682

§ 371 Date: Apr. 20, 2022

Related U.S. Application Data
(60) Provisional application No. 62/929,379, filed on Nov. 1, 2019.

(51) Int. Cl. C07K 16/28; A61P 35/02; C07K 16/22

(52) U.S. Cl. CPC C07K 16/2827; A61P 35/02; C07K 16/22; C07K 2317/565; C07K 2317/76

(57) ABSTRACT

The present invention relates to combination therapies useful for the treatment of cancer. In particular, the invention relates to the combined use of a PD-1 inhibitor, a TGFβ inhibitor, an ATM inhibitor and radiation to treat cancer.

### FIELD OF INVENTION

The present invention relates to the treatment of cancer. In particular, the invention relates to a combination of compounds for inhibiting PD-1, TGFβ and ATM for use in treating cancer together with radiotherapy.

### BACKGROUND OF THE INVENTION

Although radiation therapy is the standard of care to treat many different cancer types, treatment resistance remains a major concern. Mechanisms of resistance to radiation therapy are varied and complex, and include changes in DNA damage response pathways (DDR), modulation of immune cell functions, and increased levels of immunosuppressive cytokines like transforming growth factor beta (TGFβ). Strategies to combat resistance include combining radiation therapy with treatments that target these mechanisms.

DDR inhibitors are promising combination partners for radiation therapy. Radiation therapy kills cancer cells by damaging DNA, leading to activation of DDR pathways as cells attempt to repair the damage. Although DDR pathways are redundant in normal cells, one or more pathways is often lost during malignant progression, resulting in cancer cells relying more heavily on the remaining pathways and increasing the potential for genetic errors. This makes cancer cells uniquely vulnerable to treatment with DDR inhibitors. Since DNA double-strand breaks (DSBs) are considered the major cause of radiation-induced cell death, DDR inhibitors targeting DSB repair mechanisms may be particularly beneficial when used in combination with radiation therapy. Indeed, inhibitors of ATM, a serine/threonine kinase, involved in DSB repair, have demonstrated efficacy in sensitizing cancer cells to radiation therapy in preclinical models (T. Fuchss et al.: “Selective ATM Kinase Inhibitor M3541: A Clinical Candidate Drug with Strong Anti-Tumor Activity in Combination with Radiotherapy”; presented at AACR Annual Meeting 2018; Apr. 14-18, 2018).

Treatments targeting immunosuppressive pathways such as TGFβ and programmed death ligand 1 (PD-L1)/programmed death 1 (PD-1) are also each being investigated alone or in combination with radiation therapy. The cytokine TGFβ has a physiological role in maintaining immunological self-tolerance, but in cancer, can promote tumor growth and immune evasion through effects on innate and adaptive immunity. The immune checkpoint mediated by PD-L1/PD-1 signaling dampens T cell activity and is exploited by cancer to suppress anti-tumor T cell responses. Radiation induces expression of both PD-L1 and TGF-β, which may contribute to radiation resistance.

WO 2015/118175 describes a bifunctional fusion protein composed of the extracellular domain of the tumor growth factor beta receptor type II (TGFβRII) to function as a TGF-β “trap” fused to a human IgG1 antibody blocking PD-L1. Specifically, the protein is a heterotetramer, consisting of the two immunoglobulin light chains of an anti-PD-L1 antibody, and two heavy chains each comprising a heavy chain of the anti-PD-L1 antibody genetically fused via a flexible glycine-serine linker to the extracellular domain of the human TGFβRII (see FIG. 1). This fusion molecule is designed to target two major mechanisms of immunosuppression in the tumor microenvironment.

There remains a need to develop novel therapeutic options for the treatment of cancers. Furthermore, there is a need for therapies having greater efficacy than existing therapies. Preferred combination therapies of the present invention show greater efficacy than treatment with either therapeutic agent alone.

### SUMMARY OF THE INVENTION

Each of the embodiments described below can be combined with any other embodiment described herein not inconsistent with the embodiment with which it is combined. Furthermore, each of the embodiments described herein envisions within its scope pharmaceutically acceptable salts of the chemical compounds described herein. Accordingly, the phrase “or a pharmaceutically acceptable salt thereof” is implicit in the description of all chemical compounds described herein. Embodiments within an aspect as described below can be combined with any other embodiments not inconsistent within the same aspect or a different aspect.

The present invention arises out of the discovery that treatment outcome of radiotherapy of a subject having a cancer can be improved by further treating the subject with a combination of compounds which inhibit PD-1, TGFβ and ATM. Thus, in a first aspect, the present invention provides a PD-1 inhibitor, a TGFβ inhibitor and an ATM inhibitor for use in a method of treating a cancer in a subject, for use in inhibiting tumor growth or progression in a subject who has malignant tumors, for use in inhibiting metastasis of malignant cells in a subject, for use in decreasing the risk of metastasis development and/or metastasis growth in a subject, and for use in inducing tumor regression in a subject who has malignant cells, wherein the use comprises administering said compounds to the subject in combination with radiotherapy. The present invention also provides said PD-1 inhibitor, TGFβ inhibitor and/or ATM inhibitor for the manufacture of a medicament for the aforementioned uses, as well as methods of treatment concerning the aforementioned uses. Preferably, the PD-1 inhibitor and the TGFβ inhibitor are fused. The combination treatment together with radiotherapy results in an objective response, preferably a complete response or partial response in the subject. In some embodiments, the cancer is identified as PD-L1 positive cancerous disease.

The cancer is preferably selected from carcinoma, lymphoma, leukemia, blastoma, and sarcoma.

The PD-1 inhibitor, TGFβ inhibitor, ATM inhibitor and radiotherapy can be administered in a first-line, second-line or higher-line treatment of the cancer. In some embodiments, the cancer is resistant or became resistant to prior cancer therapy. The combination therapy of the invention can also be used in the treatment of a subject with the cancer who has been previously treated with one or more chemotherapies or underwent radiotherapy but failed with such previous treatment.

In a preferred embodiment, the subject to be treated is human.

Preferably, the PD-1 inhibitor and the TGFβ inhibitor are fused. More preferably, the fusion molecule is an anti-PD-L1/TGFβ Trap. Most preferably, the amino acid sequence of anti-PD-L1/TGFβ Trap is identical to the amino acid sequence of bintrafusp alfa.

In a further aspect, the invention also relates to a method for advertising treatment with a PD-1 inhibitor, a TGFβ inhibitor, an ATM inhibitor and radiotherapy in combination comprising promoting, to a target audience, the use of the combination for treating a subject with a cancer, e.g., based on PD-L1 expression in samples, preferably tumor samples, taken from the subject. The PD-L1 expression can be determined by immunohistochemistry, e.g., using one or more primary anti-PD-L1 antibodies.

Provided herein is also a pharmaceutical composition comprising a PD-1 inhibitor, a TGFβ inhibitor, an ATM inhibitor and at least a pharmaceutically acceptable excipient or adjuvant, wherein the PD-1 inhibitor and TGFβ inhibitor are preferably fused. The PD-1 inhibitor, the TGFβ inhibitor and the ATM inhibitor are provided in a single or separate unit dosage forms.

In a further aspect, the invention relates to a kit comprising a PD-1 inhibitor, a TGFβ inhibitor, an ATM inhibitor and a package insert comprising instructions for using said compounds together with radiotherapy to treat or delay progression of a cancer in a subject. In a further aspect, the invention relates to a kit comprising a PD-1 inhibitor and a package insert comprising instructions for using the PD-1 inhibitor, a TGFβ inhibitor, and an ATM inhibitor together with radiotherapy to treat or delay progression of a cancer in a subject. In a further aspect, the invention relates to a kit comprising a TGFβ inhibitor and a package insert comprising instructions for using the TGFβ inhibitor, a PD-1 inhibitor, and an ATM inhibitor together with radiotherapy to treat or delay progression of a cancer in a subject. In a further aspect, the invention relates to a kit comprising an ATM inhibitor and a package insert comprising instructions for using the ATM inhibitor, a PD-1 inhibitor, and a TGFβ inhibitor together with radiotherapy to treat or delay progression of a cancer in a subject. In a further aspect, the invention relates to a kit comprising an anti-PD-L1/TGFβ Trap and a package insert comprising instructions for using the anti-PD-L1/TGFβ Trap and an ATM inhibitor together with radiotherapy to treat or delay progression of a cancer in a subject. The compounds of the kit may be comprised in one or more containers. The instructions can state that the medicaments are intended for use in treating a subject having a cancer that tests positive for PD-L1 expression by an immunohistochemical (IHC) assay.

### BRIEF DESCRIPTION OF THE FIGURES

FIG. 1 shows the amino acid sequence of bintrafusp alfa. (A) SEQ ID NO: 8 represents the heavy chain sequence of bintrafusp alfa. The CDRs having the amino acid sequences of SEQ ID NOs: 1, 2 and 3 are underlined. (B) SEQ ID NO: 7 represents the light chain sequence of bintrafusp alfa. The CDRs having the amino acid sequences of SEQ ID NOs: 4, 5 and 6 are underlined.

FIG. 2 shows an exemplary structure of an anti-PD-L1/TGFβ Trap.

FIG. 3: The combination of bintrafusp alfa, radiotherapy (RT), and Compound A increased antitumor activity compared with the dual combination of bintrafusp alfa and RT. BALB/c mice were inoculated intramuscularly (i.m.) with 0.5×10⁵ 4T1 cells (day −7) and treated (n=10 mice/group) with isotype control (400 μg i.v.; day 0, 2, 4)+vehicle control (0.2 mL, orally [per os; p.o.], day 0-10), bintrafusp alfa (492 μg i.v.; day 0, 2, 4)+RT (8 Gy, day 0-3), or bintrafusp alfa+RT+Compound A (100 mg/kg, p.o, day 0-10). Tumor volumes were measured twice weekly and presented as (A) mean±SEM or (B) individual tumor volumes. P-values were calculated by two-way RM ANOVA with Tukey's or Sidak's post-test. (C) For survival analysis, mice were sacrificed when tumor volumes reached ≈2000 mm³ and median survival times were calculated.

FIG. 4: The combination of bintrafusp alfa, RT, and Compound A increased antitumor activity compared with the dual combination of bintrafusp alfa and RT, regardless of Compound A dosing schedule. BALB/c mice were inoculated intramuscularly (i.m.) with 0.5×10⁵ 4T1 cells (day −7) and treated (n=10 mice/group) with isotype control (400 μg i.v.; day 0, 2, 4)+vehicle control (0.2 mL, p.o., day 0-17), bintrafusp alfa (492 μg i.v.; day 0, 2, 4)+RT (8 Gy, day 0-3), or bintrafusp alfa+RT+Compound A (100 mg/kg, p.o, days 0-3, days 0-10, or day 0-17). Tumor volumes were measured twice weekly and presented as (A) mean±SEM or (B) individual tumor volumes. P-values were calculated by two-way RM ANOVA with Tukey's post-test. (C) For survival analysis, mice were sacrificed when tumor volumes reached ≈2000 mm³ and median survival times were calculated.

FIG. 5: The combination of bintrafusp alfa, RT, and Compound A increased antitumor activity compared with dual combinations and monotherapies. BALB/c mice were inoculated intramuscularly (i.m.) with 0.5×10⁵ 4T1 cells (day −7) and treated (n=10 mice/group) with isotype control (400 μg i.v.; day 0, 2, 4)+vehicle control (0.2 mL, p.o., day 0-4), bintrafusp alfa (492 μg i.v.; day 0, 2, 4)+vehicle control, RT (8 Gy, day 0-3)+isotype control+vehicle control, Compound A (100 mg/kg, p.o, days 0-4)+isotype control, bintrafusp alfa+RT, bintrafusp alfa+Compound A, RT+Compound A, or bintrafusp alfa+RT+Compound A. Tumor volumes were measured twice weekly and presented as (A) mean±SEM or (B) individual tumor volumes. P-values were calculated by two-way RM ANOVA with Tukey's post-test. (C) For survival analysis, mice were sacrificed when tumor volumes reached ≈2000 mm³ and median survival times were calculated.

FIG. 6: Bintrafusp alfa+Compound A+RT shows equal or better anti-tumor efficacy and survival at lower doses of RT in the 4T1 model relative to bintrafusp alfa+RT. Balb/c mice were inoculated with 4T1 cells (0.5×10⁶) in the right thigh muscle (day −7). At day 0, when average tumor volumes reached 150 mm³, mice were treated (n=10 mice/group) with isotype control (400 μg i.v.; Days 0, 2, 4), Bintrafusp alfa (492 μg/mouse i.v.; Days 0, 2, 4)+RT (8Gy, QDx4), or bintrafusp alfa+Compound A (100 mg/kg p.o., QDx4)+RT (8Gy or 6Gy or 4Gy or 2Gy, QDx4). Tumor volumes were measured twice weekly and presented as mean±SEM. P-values were calculated by two-way ANOVA with Sidack's or Tukey's post-test. P-values comparing median survival were calculated with the Log-rank (Mantel-Cox) test.

FIG. 7: Bintrafusp alfa+Compound A+RT increased the anti-tumor efficacy and survival in the MC38 model relative to the respective monotherapies and dual combination therapies. C57BL/6 mice were intramuscularly inoculated with MC38 cells (0.25×10⁵) in the right thigh muscle (day −7). At day 0, when average tumor volumes reached 50 mm³, mice were treated (n=10 mice/group) with isotype control (133 μg i.v.; Day 0), bintrafusp alfa (164 μg i.v.; Day 0), Compound A (100 mg/kg p.o.; QDx4), RT (1.8Gy; QDx4), bintrafusp alfa+RT, bintrafusp alfa+Compound A, RT+Compound A or bintrafusp alfa+Compound A+RT. Tumor volumes were measured twice weekly and presented as mean±SEM or as individual tumor volumes. P-values were calculated by two-way ANOVA with Tukey's or Sidak's post-test.

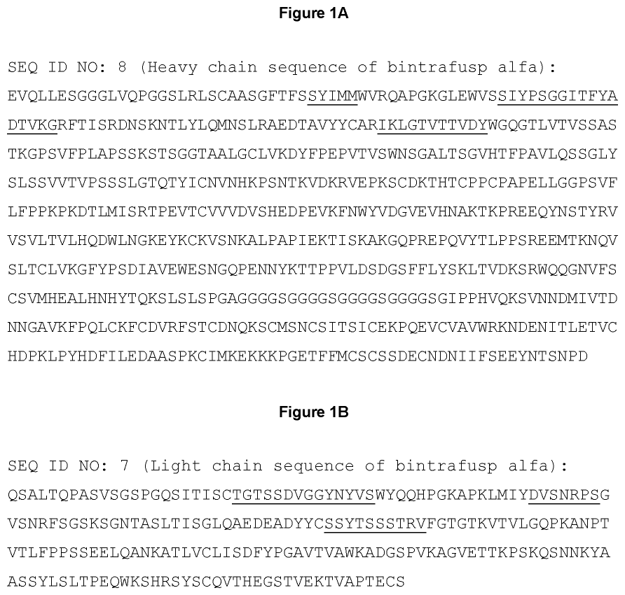

### DETAILED DESCRIPTION OF THE INVENTION

### Definitions

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

“A”, “an”, and “the” include plural referents unless the context clearly dictates otherwise. Thus, for example, reference to an antibody refers to one or more antibodies or at least one antibody. As such, the terms “a” (or “an”), “one or more”, and “at least one” are used interchangeably herein.

“About” when used to modify a numerically defined parameter (e.g., the dose of a PD-1 inhibitor, a TGFβ inhibitor or an ATM inhibitor, or the length of treatment time with a combination therapy described herein) means that the parameter may vary by as much as 10% below or above the stated numerical value for that parameter. For example, a dose of about 10 mg/kg may vary between 9 mg/kg and 11 mg/kg.

“Administering” or “administration of” a drug to a patient (and grammatical equivalents of this phrase) refers to direct administration, which may be administration to a patient by a medical professional or may be self-administration, and/or indirect administration, which may be the act of prescribing a drug, e.g., a physician who instructs a patient to self-administer a drug or provides a patient with a prescription for a drug is administering the drug to the patient.

“Antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also, unless otherwise specified, any antigen-binding fragment or antibody fragment thereof that competes with the intact antibody for specific binding, as well as any protein comprising such antigen-binding fragment or antibody fragment thereof, including fusion proteins (e.g., antibody-drug conjugates, an antibody fused to a cytokine or an antibody fused to a cytokine receptor), antibody compositions with poly-epitopic specificity, and multi-specific antibodies (e.g., bispecific antibodies).

“Antigen-binding fragment” of an antibody or “antibody fragment” comprises a portion of an intact antibody, which is still capable of antigen binding. Antigen-binding fragments include, for example, Fab, Fab′, F(ab′)₂, Fd, and Fv fragments, domain antibodies (dAbs, e.g., shark and camelid antibodies), fragments including complementarity determining regions (CDRs), single chain variable fragment antibodies (scFv), single-chain antibody molecules, multi-specific antibodies formed from antibody fragments, maxibodies, nanobodies, minibodies, intrabodies, diabodies, triabodies, tetrabodies, v-NAR and bis-scFv, linear antibodies (see e.g., U.S. Pat. No. 5,641,870, Example 2; Zapata et al. (1995) Protein Eng. 8HO: 1057), and polypeptides that contain at least a portion of an immunoglobulin that is sufficient to confer specific antigen binding to the polypeptide. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment, which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments were originally produced as pairs of Fab′ fragments which have hinge cysteines between them. Other chemical couplings of antibody fragments are also known.

“Antibody-dependent cell-mediated cytotoxicity” or “ADCC” refers to a form of cytotoxicity in which secreted Ig bound onto Fc receptors (FcRs) present on certain cytotoxic cells (e.g., natural killer (NK) cells, neutrophils, and macrophages) enable these cytotoxic effector cells to bind specifically to an antigen-bearing target cell and subsequently kill the target cell with cytotoxins. The antibodies arm the cytotoxic cells and are required for killing of the target cell by this mechanism. The primary cells for mediating ADCC, the NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991).

“Anti-PD-L1 antibody” or “anti-PD-1 antibody” means an antibody, or an antigen-binding fragment thereof, that blocks binding of PD-L1 expressed on a cancer cell to PD-1. In any of the treatment methods, medicaments and uses of the present invention in which a human subject is being treated, the anti-PD-L1 antibody specifically binds to human PD-L1 and blocks binding of human PD-L1 to human PD-1. In any of the treatment methods, medicaments and uses of the present invention in which a human subject is being treated, the anti-PD-1 antibody specifically binds to human PD-1 and blocks binding of human PD-L1 to human PD-1. The antibody may be a monoclonal antibody, human antibody, humanized antibody or chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen-binding fragment is selected from the group consisting of Fab, Fab′-SH, F(ab′)2, scFv and Fv fragments.

“Anti-PD-L1/TGFβ Trap” refers to a fusion molecule of the PD-1 inhibitor and the TGFβ inhibitor comprising (1) an antibody or a fragment thereof capable of binding PD-L1 and inhibiting the interaction between PD-1 and PD-L1 and (2) the extracellular domain of TGFβRII or a fragment thereof capable of binding TGFβ and inhibiting the interaction between TGFβ and a TGFβ receptor.

“ATM inhibitor” as used herein refers to a molecule that inhibits the ATM signaling pathway, preferably by inhibiting the activity of the ATM kinase. Preferably, the ATM inhibitor is Compound A, or a pharmaceutically acceptable salt thereof.

“Biomarker” generally refers to biological molecules, and quantitative and qualitative measurements of the same, that are indicative of a disease state. “Prognostic biomarkers” correlate with disease outcome, independent of therapy. For example, tumor hypoxia is a negative prognostic marker—the higher the tumor hypoxia, the higher the likelihood that the outcome of the disease will be negative. “Predictive biomarkers” indicate whether a patient is likely to respond positively to a particular therapy. E.g., HER2 profiling is commonly used in breast cancer patients to determine if those patients are likely to respond to Herceptin (trastuzumab, Genentech). “Response biomarkers” provide a measure of the response to a therapy and so provide an indication of whether a therapy is working. For example, decreasing levels of prostate-specific antigen generally indicate that anti-cancer therapy for a prostate cancer patient is working. When a marker is used as a basis for identifying or selecting a patient for a treatment described herein, the marker can be measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of a biomarker in a clinical setting is a clear indication that this parameter was used as a basis for initiating, continuing, adjusting and/or ceasing administration of the treatments described herein.

“Blood” refers to all components of blood circulating in a subject including, but not limited to, red blood cells, white blood cells, plasma, clotting factors, small proteins, platelets and/or cryoprecipitate. This is typically the type of blood which is donated when a human patient gives blood. Plasma is known in the art as the yellow liquid component of blood, in which the blood cells in whole blood are typically suspended. It makes up about 55% of the total blood volume. Blood plasma can be prepared by spinning a tube of fresh blood containing an anti-coagulant in a centrifuge until the blood cells fall to the bottom of the tube. The blood plasma is then poured or drawn off. Blood plasma has a density of approximately 1025 kg/m³ or 1.025 kg/I.

“Cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, acute myeloid leukemia, multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma, cervical cancer, brain cancer, stomach cancer, bladder cancer, hepatoma, breast cancer, colon carcinoma, biliary tract cancer, and head and neck cancer. “Chemotherapy” is a therapy involving a chemotherapeutic agent, which is a chemical compound useful in the treatment of cancer. Examples of chemotherapeutic agents include alkylating agents such as thiotepa and cyclophosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide, and trimethylolomelamine; acetogenins (especially bullatacin and bullatacinone); delta-9-tetrahydrocannabinol (dronabinol); beta-lapachone; lapachol; colchicines; betulinic acid; a camptothecin (including the synthetic analogue topotecan (CPT-11 (irinotecan), acetylcamptothecin, scopolectin, and 9-aminocamptothecin); bryostatin; pemetrexed; callystatin; CC-1065 (including its adozelesin, carzelesin, and bizelesin synthetic analogues); podophyllotoxin; podophyllinic acid; teniposide; cryptophycins (particularly, cryptophycin 1 and cryptophycin 8); dolastatin; duocarmycin (including the synthetic analogues KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Nicolaou et al. (1994) Angew. Chem Intl. Ed. Engl. 33: 183); dynemicin including dynemicin A; an esperamicin; as well as neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, doxorubicin HCl liposome injection, and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites such as methotrexate, gemcitabine, tegafur, capecitabine, an epothilone, and 5-fluorouracil (5-FU); folic acid analogues such as denopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, and trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially, T-2 toxin, verracurin A, roridin A, and anguidine); urethan; vindesine; dacarbazine; mannomustine; mitobronitol; mitolactol; pipobroman; gacytosine; arabinoside (“Ara-C”); thiotepa; taxoids, e.g., paclitaxel, albumin-engineered nanoparticle formulation of paclitaxel, and doxetaxel; chloranbucil; 6-thioguanine; mercaptopurine; methotrexate; platinum analogs such as cisplatin and carboplatin; vinblastine; platinum; etoposide (VP-16); ifosfamide; mitoxantrone; vincristine; oxaliplatin; leucovovin; vinorelbine; novantrone; edatrexate; daunomycin; aminopterin; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine and prednisolone, or FOLFOX, an abbreviation for a treatment regimen with oxaliplatin combined with 5-FU and leucovovin.

“Clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity, or side effect.

“Combination” as used herein refers to the provision of a first active modality in addition to one or more further active modalities (wherein one or more active modalities may be fused). Contemplated within the scope of the combinations described herein, are any regimen of combination modalities or partners (i.e., active compounds, components or agents), such as a combination of a PD-1 inhibitor, a TGFβ inhibitor and an ATM inhibitor, encompassed in single or multiple compounds and compositions. It is understood that any modalities within a single composition, formulation or unit dosage form (i.e., a fixed-dose combination) must have the identical dose regimen and route of delivery. It is not intended to imply that the modalities must be formulated for delivery together (e.g., in the same composition, formulation or unit dosage form). The combined modalities can be manufactured and/or formulated by the same or different manufacturers. The combination partners may thus be, e.g., entirely separate pharmaceutical dosage forms or pharmaceutical compositions that are also sold independently of each other. Preferably, the TGFβ inhibitor is fused to the PD-1 inhibitor and therefore encompassed within a single composition and having an identical dose regimen and route of delivery.

“Combination therapy”, “in combination with” or “in conjunction with” as used herein denotes any form of concurrent, parallel, simultaneous, sequential or intermittent treatment with at least two distinct treatment modalities (i.e., compounds, components, targeted agents or therapeutic agents). As such, the terms refer to administration of one treatment modality before, during, or after administration of the other treatment modality to the subject. The modalities in combination can be administered in any order. The therapeutically active modalities are administered together (e.g., simultaneously in the same or separate compositions, formulations or unit dosage forms) or separately (e.g., on the same day or on different days and in any order as according to an appropriate dosing protocol for the separate compositions, formulations or unit dosage forms) in a manner and dosing regimen prescribed by a medical care taker or according to a regulatory agency. In general, each treatment modality will be administered at a dose and/or on a time schedule determined for that treatment modality. Optionally, four or more modalities may be used in a combination therapy. Additionally, the combination therapies provided herein may be used in conjunction with other types of treatment. For example, other anti-cancer treatment may be selected from the group consisting of chemotherapy, surgery, radiotherapy (radiation) and/or hormone therapy, amongst other treatments associated with the current standard of care for the subject.

“Complete response” or “complete remission” refers to the disappearance of all signs of cancer in response to treatment. This does not always mean the cancer has been cured.

“Comprising”, as used herein, is intended to mean that the compositions and methods include the recited elements, but not excluding others. “Consisting essentially of”, when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

“Dose” and “dosage” refer to a specific amount of active or therapeutic agents for administration. Such amounts are included in a “dosage form,” which refers to physically discrete units suitable as unitary dosages for human subjects and other mammals, each unit containing a predetermined quantity of active agent calculated to produce the desired onset, tolerability, and therapeutic effects, in association with one or more suitable pharmaceutical excipients such as carriers.

“Enhancing T-cell function” means to induce, cause or stimulate a T-cell to have a sustained or amplified biological function, or renew or reactivate exhausted or inactive T-cells. Examples of enhancing T-cell function include: increased secretion of y-interferon from CD8+ T-cells, increased proliferation, increased antigen responsiveness (e.g., viral, pathogen, or tumor clearance) relative to such levels before the intervention. In one embodiment, the level of enhancement is as least 50%, alternatively 60%, 70%, 80%, 90%, 100%, 120%, 150%, 200%. The manner of measuring this enhancement is known to one of ordinary skill in the art.

“Fc” is a fragment comprising the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment, which contains a complete antigen-recognition and antigen-binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen-binding specificity to the antibody. However, even a single variable domain (or half of an Fv comprising only three HVRs specific for an antigen) has the ability to recognize and bind antigen, although at a lower affinity than the entire binding site.

“Human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (see e.g., Hoogenboom and Winter (1991), JMB 227: 381; Marks et al. (1991) JMB 222: 581). Also available for the preparation of human monoclonal antibodies are methods described in Cole et al. (1985) Monoclonal Antibodies and Cancer Therapy, Alan R. Liss, page 77; Boerner et al. (1991), J. Immunol 147(l): 86; van Dijk and van de Winkel (2001) Curr. Opin. Pharmacol 5: 368). Human antibodies can be prepared by administering the antigen to a transgenic animal that has been modified to produce such antibodies in response to antigenic challenge but whose endogenous loci have been disabled, e.g., immunized xenomice (see e.g., U.S. Pat. Nos. 6,075,181; and 6,150,584 regarding XENOMOUSE technology). See also, for example, Li et al. (2006) PNAS USA, 103: 3557, regarding human antibodies generated via a human B-cell hybridoma technology.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit, or non-human primate having the desired specificity, affinity and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and no more than 3 in the L chain. The humanized antibody optionally will also comprise at least a portion of an immunoglobulin constant region (Fc), typically that of a human immunoglobulin. For further details, see e.g., Jones et al. (1986) Nature 321: 522; Riechmann et al. (1988), Nature 332: 323; Presta (1992) Curr. Op. Struct. Biol. 2: 593; Vaswani and Hamilton (1998), Ann. Allergy, Asthma & Immunol. 1: 105; Harris (1995) Biochem. Soc. Transactions 23: 1035; Hurle and Gross (1994) Curr. Op. Biotech. 5: 428; and U.S. Pat. Nos. 6,982,321 and 7,087,409.

“Immunoglobulin” (Ig) is used interchangeably with “antibody” herein. The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intra-chain disulfide bridges. Each H chain has, at the N-terminus, a variable domain (V_{H}) followed by three constant domains (C_{H}) for each of the α and γ chains and four C_{H} domains for p and E isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8^{th} Edition, Sties et al. (eds.), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes.

There are five classes of immunoglobulins: IgA, IgD, IgE, IgG and IgM, having heavy chains designated α, δ, ε, γ and μ, respectively. The γ and a classes are further divided into subclasses on the basis of relatively minor differences in the C_{H} sequence and function, e.g., humans express the following subclasses: IgG1, IgG2A, IgG2B, IgG3, IgG4, IgA1, and IgK1.

“Infusion” or “infusing” refers to the introduction of a drug-containing solution into the body through a vein for therapeutic purposes. Generally, this is achieved via an intravenous (IV) bag.

“Isolated” refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues. For example, an “isolated antibody” is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. The “isolated antibody” includes the antibody in-situ within recombinant cells since at least one component of the antibody's natural environment will not be present. Ordinarily, however, an isolated polypeptide or antibody will be prepared by at least one purification step.

“Metastatic” cancer refers to cancer which has spread from one part of the body (e.g., the lung) to another part of the body.

“Monoclonal antibody”, as used herein, refers to an antibody obtained from a population of substantially homogeneous antibodies, i.e., the individual antibodies comprising the population are identical except for possible naturally occurring mutations and/or post-translation modifications (e.g., isomerizations and amidations) that may be present in minor amounts. Monoclonal antibodies are highly specific, being directed against a single antigenic site. In contrast to polyclonal antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture and uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein (1975) Nature 256: 495; Hongo et al. (1995) Hybridoma 14 (3): 253; Harlow et al. (1988) Antibodies: A Laboratory Manual (Cold Spring Harbor Laboratory Press, 2^{nd} ed.; Hammerling et al. (1981) In: Monoclonal Antibodies and T-Cell Hybridomas 563 (Elsevier, N.Y.), recombinant DNA methods (see e.g., U.S. Pat. No. 4,816,567), phage-display technologies (see e.g., Clackson et al. (1991) Nature 352: 624; Marks et al. (1992) JMB 222: 581; Sidhu et al. (2004) JMB 338(2): 299; Lee et al. (2004) JMB 340(5): 1073; Fellouse (2004) PNAS USA 101(34): 12467; and Lee et al. (2004) J. Immunol. Methods 284(1-2): 119), and technologies for producing human or human-like antibodies in animals that have parts or all of the human immunoglobulin loci or genes encoding human immunoglobulin sequences (see e.g., WO 1998/24893; WO 1996/34096; WO 1996/33735; WO 1991/10741; Jakobovits et al. (1993) PNAS USA 90: 2551; Jakobovits et al. (1993) Nature 362: 255; Bruggemann et al. (1993) Year in Immunol. 7: 33; U.S. Pat. Nos. 5,545,807; 5,545,806; 5,569,825; 5,625,126; 5,633,425; and U.S. Pat. No. 5,661,016; Marks et al. (1992) Bio/Technology 10: 779; Lonberg et al. (1994) Nature 368: 856; Morrison (1994) Nature 368: 812; Fishwild et al. (1996) Nature Biotechnol. 14: 845; Neuberger (1996), Nature Biotechnol. 14: 826; and Lonberg and Huszar (1995), Intern. Rev. Immunol. 13: 65-93). The monoclonal antibodies herein specifically include chimeric antibodies (immunoglobulins) in which a portion of the heavy and/or light chain is identical to or homologous to corresponding sequences in antibodies derived from a particular species or belonging to a particular antibody class or subclass, while the remainder of the chain(s) is (are) identical to or homologous to corresponding sequences in antibodies derived from another species or belonging to another antibody class or subclass, as well as fragments of such antibodies, so long as they exhibit the desired biological activity (see e.g., U.S. Pat. No. 4,816,567; Morrison et al. (1984) PNAS USA, 81: 6851).

“Objective response” refers to a measurable response, including complete response (CR) or partial response (PR).

“Partial response” refers to a decrease in the size of one or more tumors or lesions, or in the extent of cancer in the body, in response to treatment.

“Patient” and “subject” are used interchangeably herein to refer to a mammal in need of treatment for a cancer. Generally, the patient is a human diagnosed or at risk for suffering from one or more symptoms of a cancer. In certain embodiments a “patient” or “subject” may refer to a non-human mammal, such as a non-human primate, a dog, cat, rabbit, pig, mouse, or rat, or animals used, e.g., in screening, characterizing, and evaluating drugs and therapies.

“PD-1 inhibitor” as used herein refers to a molecule that inhibits the PD-1 pathway, preferably by inhibiting the interaction of PD-1 axis binding partners, such as PD-L1 and PD-1. Possible effects of such inhibition include the removal of T-cell dysfunction resulting from signaling on the PD-1 signaling axis. Preferably, the PD-1 inhibitor binds to PD-L1 or PD-1 to inhibit the interaction between these molecules, such as an anti-PD-1 antibody or an anti-PD-L1 antibody. Preferably, the PD-1 inhibitor is a PD-L1 antibody and, more preferably, such antibody is fused to the TGFβ inhibitor, such as the anti-PD-L1/TGFβ Trap molecule.

“PD-L1 expression” as used herein means any detectable level of expression of PD-L1 protein on the cell surface or of PD-L1 mRNA within a cell or tissue. PD-L1 protein expression may be detected with a diagnostic PD-L1 antibody in an IHC assay of a tumor tissue section or by flow cytometry. Alternatively, PD-L1 protein expression by tumor cells may be detected by PET imaging, using a binding agent (e.g., antibody fragment, affibody and the like) that specifically binds to PD-L1. Techniques for detecting and measuring PD-L1 mRNA expression include RT-PCR and real-time quantitative RT-PCR.

“PD-L1 positive” cancer, including a “PD-L1 positive” cancerous disease, is one comprising cells, which have PD-L1 present at their cell surface. The term “PD-L1 positive” also refers to a cancer that produces sufficient levels of PD-L1 at the surface of cells thereof, such that an anti-PD-L1 antibody has a therapeutic effect, mediated by the binding of the said anti-PD-L1 antibody to PD-L1. Methods of detecting a biomarker, such as PD-L1 for example, on a cancer or tumor, are routine in the art and are contemplated herein. Non-limiting examples include immunohistochemistry (IHC), immunofluorescence and fluorescence activated cell sorting (FACS). Several approaches have been described for quantifying PD-L1 protein expression in IHC assays of tumor tissue sections. The ratio of PD-L1 positive cells is oftentimes expressed as a Tumor Proportion Score (TPS) or a Combined Positive Score (CPS). The TPS describes the percentage of viable tumor cells with partial or complete membrane staining (e.g., staining for PD-L1). The CPS is the number of PD-L1 staining cells (tumor cells, lymphocytes, macrophages) divided by the total number of viable tumor cells, multiplied by 100. For instance, in some embodiments, “PD-L1 high” refers to 80% PD-L1 positive tumor cells as determined by the PD-L1 Dako IHC 73-10 assay, or tumor proportion score (TPS) 50% as determined by the Dako IHC 22C3 PharmDx assay. Both IHC 73-10 and IHC 22C3 assays select a similar patient population at their respective cutoffs. In certain embodiments, Ventana PD-L1 (SP263) assay, which has high concordance with 22C3 PharmDx assay (see Sughayer et al., Appl. Immunohistochem. Mol. Morphol., (2018)), can also be used for determining the PD-L1 expression level. Another assay for determining PD-L1 expression in cancers is the Ventana PD-L1 (SP142) assay. In some embodiments, a cancer is counted as PD-L1 positive if at least 1%, at least 5%, at least 25%, at least 50%, at least 75% or at least 80% of the tumor cells show PD-L1 expression.

“Pharmaceutically acceptable” indicates that the substance or composition must be compatible chemically and/or toxicologically, with the other ingredients comprising a formulation, and/or the mammal being treated therewith. “Pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof.

“Recurrent” cancer is one which has regrown, either at the initial site or at a distant site, after a response to initial therapy, such as surgery. A locally “recurrent” cancer is cancer that returns after treatment in the same place as a previously treated cancer.

“Reduction” of a symptom or symptoms (and grammatical equivalents of this phrase) refers to decreasing the severity or frequency of the symptom(s), or elimination of the symptom(s).

“Serum” refers to the clear liquid that can be separated from clotted blood. Serum differs from plasma, the liquid portion of normal unclotted blood containing the red and white cells and platelets. Serum is the component that is neither a blood cell (serum does not contain white or red blood cells) nor a clotting factor. It is the blood plasma not including the fibrinogens that help in the formation of blood clots. It is the clot that makes the difference between serum and plasma.

“Single-chain Fv”, also abbreviated as “sFv” or “scFv”, are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see e.g., Pluckthun (1994), In: The Pharmacology of Monoclonal Antibodies, vol. 113, Rosenburg and Moore (eds.), Springer-Verlag, New York, pp. 269.

By “substantially identical” is meant (1) a polypeptide exhibiting at least 75%, desirably 85%, 90%, or 95%, and more desirably 99% amino acid sequence identity to a reference amino acid sequence or (2) a polypeptide that differs in not more than 40% of its amino acid positions, desirably in not more than 30% of its amino acid positions, and more desirably in not more than 20% of its amino acid positions from the amino acid sequence of a reference amino acid sequence and wherein a difference in an amino acid position is any of a substitution, deletion or addition of an amino acid. The length of comparison sequences for determining the degree of sequence identity will generally be at least 20 or 25 contiguous amino acids, more desirably at least 50, 75, 90, 100, 150, 200, 250, 300, or 350 contiguous amino acids, and most desirably the full-length amino acid sequence.

“Suitable for therapy” or “suitable for treatment” shall mean that the patient is likely to exhibit one or more desirable clinical outcomes as compared to patients having the same cancer and receiving the same therapy but possessing a different characteristic that is under consideration for the purpose of the comparison. In one aspect, the characteristic under consideration is a genetic polymorphism or a somatic mutation (see e.g., Samsami et al. (2009) J Reproductive Med 54(1): 25). In another aspect, the characteristic under consideration is the expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, may exhibit more than one more desirable clinical outcomes as compared to patients having the same cancer and receiving the same therapy but not possessing the characteristic. As defined herein, the patient is considered suitable for the therapy. In another such aspect, a patient possessing a characteristic may exhibit one or more desirable clinical outcomes but simultaneously exhibit one or more less desirable clinical outcomes. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Systemic” treatment is a treatment, in which the drug substance travels through the bloodstream, reaching and affecting cells all over the body.

“TGFβ inhibitor” as used herein refers to a molecule that inhibits the TGFβ pathway, preferably by inhibiting the interaction between a TGFβ and a TGFβ receptor (TGFβR). Preferably, the TGFβ inhibitor binds to TGFβ or a TGFβR to inhibit the interaction between these molecules. Preferably, the TGFβ inhibitor comprises the extracellular domain of a TGFβRII, or a fragment of TGFβRII capable of binding TGFβ. Preferably, such TGFβ inhibitor is fused to the PD-1 inhibitor, e.g., as an anti-PD-L1/TGFβ Trap.

By “TGFβRII” or “TGFβ Receptor II” is meant a polypeptide having the wild-type human TGFβ Receptor Type 2 Isoform A sequence (e.g., the amino acid sequence of NCBI Reference Sequence (RefSeq) Accession No. NP_001020018 (SEQ ID NO: 9)), or a polypeptide having the wild-type human TGFβ Receptor Type 2 Isoform B sequence (e.g., the amino acid sequence of NCBI RefSeq Accession No. NP_003233 (SEQ ID NO: 10)) or having a sequence substantially identical to the amino acid sequence of SEQ ID NO: 9 or of SEQ ID NO: 10 that retains at least 25%, 50%, 75%, 90%, 95%, or 99% of the TGFβ-binding activity of the wild-type sequence. The polypeptide of expressed TGFβRII lacks the signal sequence.

By a “fragment of TGFβRII capable of binding TGFβ” is meant any portion of NCBI RefSeq Accession No. NP_001020018 (SEQ ID NO: 9) or of NCBI RefSeq Accession No. NP_003233 (SEQ ID NO: 10), or a sequence substantially identical to SEQ ID NO: 9 or SEQ ID NO: 10 that is at least 20 (e.g., at least 30, 40, 50, 60, 70, 80, 90, 100, 110, 120, 130, 140, 150, 160, 175, or 200) amino acids in length that retains at least some of the TGFβ-binding activity (e.g., at least 25%, 50%, 75%, 90%, 95%, or 99%) of the wild-type receptor or of the corresponding wild-type fragment. Typically, such fragment is a soluble fragment. In some embodiments, the fragment of TGFβRII is selected from the group consisting of SEQ ID NO: 11, SEQ ID NO: 12 and SEQ ID NO: 13.

“TGFβ expression” as used herein means any detectable level of expression of TGFβ protein or TGFβ mRNA within a cell or tissue. TGFβ protein expression may be detected with a diagnostic TGFβ antibody in an IHC assay of a tumor tissue section or by flow cytometry. Alternatively, TGFβ protein expression by tumor cells may be detected by PET imaging, using a binding agent (e.g., antibody fragment, affibody and the like) that specifically binds to TGFβ. Techniques for detecting and measuring TGFβ mRNA expression include RT-PCR and real-time quantitative RT-PCR.

“TGFβ positive” cancer, including a “TGFβ positive” cancerous disease, is one comprising cells, which secrete TGFβ. The term “TGFβ positive” also refers to a cancer that produces sufficient levels of TGFβ in the cells thereof, such that an TGFβ inhibitor has a therapeutic effect.

“Therapeutically effective amount” of a PD-1 inhibitor, a TGFβ inhibitor, an ATM inhibitor, or radiotherapy, in each case of the invention, refers to an amount effective, at dosages and for periods of time necessary, that, when administered to a patient with a cancer, will have the intended therapeutic effect, e.g., alleviation, amelioration, palliation, or elimination of one or more manifestations of the cancer in the patient, or any other clinical result in the course of treating a cancer patient. A therapeutic effect does not necessarily occur by administration of one dose, and may occur only after administration of a series of doses. Thus, a therapeutically effective amount may be administered in one or more administrations. Such therapeutically effective amount may vary according to factors such as the disease state, age, sex, and weight of the individual, and the ability of a PD-1 inhibitor, a TGFβ inhibitor, an ATM inhibitor, or radiotherapy to elicit a desired response in the individual. A therapeutically effective amount is also one in which any toxic or detrimental effects of a PD-1 inhibitor, a TGFβ inhibitor, an ATM inhibitor, or radiotherapy are outweighed by the therapeutically beneficial effects.

“Treating” or “treatment of” a condition or patient refers to taking steps to obtain beneficial or desired results, including clinical results. For purposes of this invention, beneficial or desired clinical results include, but are not limited to, alleviation, amelioration of one or more symptoms of a cancer; diminishment of extent of disease; delay or slowing of disease progression; amelioration, palliation, or stabilization of the disease state; or other beneficial results. It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors.

“Unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

“Variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

As used herein, a plurality of items, structural elements, compositional elements, and/or materials may be presented in a common list for convenience. However, these lists should be construed as though each member of the list is individually identified as a separate and unique member.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

### Descriptive Embodiments

Therapeutic Combination and Method of Use Thereof

The present invention arose in part from the surprising discovery of a combination benefit for an ATM inhibitor, a PD-1 inhibitor, a TGFβ inhibitor, and radiotherapy. Treatment schedule and doses were designed to reveal potential synergies. Pre-clinical data demonstrated a synergy of the ATM inhibitor, particularly Compound A, in combination with the PD-1 inhibitor and the TGFβ inhibitor, particularly fused as the bintrafusp alfa molecule, together with radiotherapy.

Thus, in one aspect, the present invention provides the use of a PD-1 inhibitor, a TGFβ inhibitor, an ATM inhibitor, and radiotherapy in a method for treating a cancer in a subject in need thereof, comprising administering to the subject the PD-1 inhibitor, TGFβ inhibitor, ATM inhibitor, and radiotherapy. It shall be understood that a therapeutically effective amount of the PD-1 inhibitor, TGFβ inhibitor, ATM inhibitor and radiotherapy is applied in each method of treatment. Preferably, the PD-1 inhibitor is fused to the TGFβ inhibitor. More preferably, such fusion molecule is an anti-PD-L1/TGFβ Trap, e.g., an anti-PD-L1/TGFβ Trap wherein the light chain sequences and the heavy chain sequences respectively correspond to SEQ ID NO: 7 and SEQ ID NO: 8, SEQ ID NO: 15 and SEQ ID NO: 17, or SEQ ID NO: 15 and SEQ ID NO: 18. Most preferably, the light chain sequences and the heavy chain sequences of anti-PD-L1/TGFβ Trap correspond to SEQ ID NO: 7 and SEQ ID NO: 8. respectively.

The PD-1 inhibitor preferably inhibits the interaction between PD-1 and at least one of its ligands, such as PD-L1 or PD-L2, and thereby inhibits the PD-1 pathway, e.g., the immunosuppressive signal of PD-1. The PD-1 inhibitor may bind to PD-1 or one of its ligands, such as PD-L1. In a preferred embodiment, the PD-1 inhibitor inhibits the interaction between PD-1 and PD-L1. In some embodiments, the PD-1 inhibitor is an anti-PD-1 antibody or an anti-PD-L1 antibody capable of inhibiting the interaction between PD-1 and PD-L1. In some embodiments, the anti-PD-1 antibody or anti-PD-L1 antibody is selected from the group consisting of pembrolizumab, nivolumab, avelumab, atezolizumab, durvalumab, spartalizumab, camrelizumab, sintilimab, tislelizumab, toripalimab, cemiplimab, and an antibody wherein the light chains and the heavy chains of the antibody correspond to SEQ ID NO: 7 and SEQ ID NO: 16, or to SEQ ID NO: 15 and SEQ ID NO: 14, respectively, or an antibody that competes for binding with any of the antibodies of this group. In some embodiments, the anti-PD-1 antibody or anti-PD-L1 antibody is one that is still capable of binding to PD-1 or PD-L1 and which amino acid sequence is substantially identical to the sequence of one of the antibodies selected from the group consisting of pembrolizumab, nivolumab, avelumab, atezolizumab, durvalumab, spartalizumab, camrelizumab, sintilimab, tislelizumab, toripalimab, cemiplimab, and an antibody wherein the light chains and the heavy chains of the antibody correspond to SEQ ID NO: 7 and SEQ ID NO: 16, or to SEQ ID NO: 15 and SEQ ID NO: 14, respectively.

In some embodiments, the PD-1 inhibitor is an anti-PD-L1 antibody, wherein each of the light and heavy chain sequences have greater than or equal to 80% sequence identity, such as greater than or equal to 90% sequence identity, greater than or equal to 95% sequence identity, greater than or equal to 99% sequence identity, or 100% sequence identity with the amino acid sequence of the heavy and light chains of the antibody moiety of bintrafusp alfa and wherein the PD-1 inhibitor is still capable of binding to PD-L1. In some embodiments, the PD-1 inhibitor is an anti-PD-L1 antibody, wherein each of the light and heavy chain sequences have greater than or equal to 80% sequence identity, such as greater than or equal to 90% sequence identity, greater than or equal to 95% sequence identity, greater than or equal to 99% sequence identity, or 100% sequence identity with the amino acid sequence of the heavy and light chains of the antibody moiety of bintrafusp alfa and wherein the CDRs are fully identical with the CDRs of bintrafusp alfa. In some embodiments, the PD-1 inhibitor is an anti-PD-L1 antibody with an amino acid sequence with not more than 50, not more than 40, not more than 25, or not more than 10 amino acid residues different from each of the heavy and light chain sequences of the antibody moiety of bintrafusp alfa and wherein the PD-1 inhibitor is still capable of binding to PD-L1. In some embodiments, the PD-1 inhibitor is an anti-PD-L1 antibody with an amino acid sequence with not more than 50, not more than 40, not more than 25, or not more than 10 amino acid residues different from each of the heavy and light chain sequences of the antibody moiety of bintrafusp alfa and wherein the CDRs are fully identical with the CDRs of bintrafusp alfa.

Preferably, the PD-1 inhibitor is an anti-PD-L1 antibody capable of inhibiting the interaction between PD-1 and PD-L1. In some embodiments, the anti-PD-L1 antibody comprises a heavy chain, which comprises three CDRs having amino acid sequences of SEQ ID NO: 19 (CDR1), SEQ ID NO: 20 (CDR2) and SEQ ID NO: 21 (CDR3), and a light chain, which comprises three CDRs having amino acid sequences of SEQ ID NO: 22 (CDR1), SEQ ID NO: 23 (CDR2) and SEQ ID NO: 24 (CDR3). In preferred embodiments, the anti-PD-L1 antibody comprises a heavy chain, which comprises three CDRs having amino acid sequences of SEQ ID NOs: 1, 2 and 3, and a light chain, which comprises three CDRs having amino acid sequences of SEQ ID NOs: 4, 5 and 6. In some embodiments, the light chain variable region and the heavy chain variable region of the anti-PD-L1 antibody comprise SEQ ID NO: 25 and SEQ ID NO: 26, respectively.

Preferably, the light chains and the heavy chains of the anti-PD-L1 antibody correspond to SEQ ID NO: 7 and SEQ ID NO: 16, or to SEQ ID NO: 15 and SEQ ID NO: 14, respectively. Preferably, the TGFβ inhibitor is capable of inhibiting the interaction between TGFβ and a TGFβ receptor; such as a TGFβ receptor, a TGFβ ligand- or receptor-blocking antibody, a small molecule inhibiting the interaction between TGFβ binding partners, and an inactive mutant TGFβ ligand that binds to the TGFβ receptor and competes for binding with endogenous TGFβ. Preferably, the TGFβ inhibitor is a soluble TGFβ receptor (e.g., a soluble TGFβ receptor II or Ill) or a fragment thereof capable of binding TGFβ. More preferably, the TGFβ inhibitor is an extracellular domain of human TGFβ receptor II (TGFβRII), or fragment thereof capable of binding TGFβ. In some embodiments, the TGFβRII corresponds to the wild-type human TGF-β Receptor Type 2 Isoform A sequence (e.g. the amino acid sequence of NCBI Reference Sequence (RefSeq) Accession No. NP_001020018 (SEQ ID NO: 9)), or the wild-type human TGF-β Receptor Type 2 Isoform B sequence (e.g., the amino acid sequence of NCBI RefSeq Accession No. NP_003233 (SEQ ID NO: 10)). Preferably, the TGFβ inhibitor comprises or consists of a sequence corresponding to SEQ ID NO: 11 or a fragment thereof capable of binding TGFβ. For instance, the TGFβ inhibitor may correspond to the full-length sequence of SEQ ID NO: 11. Alternatively, it may have an N-terminal deletion. For instance, the N-terminal 26 or less amino acids of SEQ ID NO: 11 may be deleted, such as 14-21 or 14-26 N-terminal amino acids. In some embodiments, the N-terminal 14, 19 or 21 amino acids of SEQ ID NO: 11 are deleted. Preferably, the TGFβ inhibitor comprises or consists of a sequence selected from the group consisting of SEQ ID NO: 11, SEQ ID NO: 12 and SEQ ID NO: 13. Preferably, the TGFβ inhibitor has at least 80%, preferably 90%, more preferably 95%, sequence identity to the full-length amino acid sequence of any one of SEQ ID NO: 11, SEQ ID NO: 12 and SEQ ID NO: 13 and is capable of binding TGFβ. In another preferred embodiment, the TGFβ inhibitor has at least 80% sequence identity to the full-length amino acid sequence of SEQ ID NO: 11 and is capable of binding TGFβ. In a preferred embodiment, the TGFβ inhibitor has an amino acid sequence that does not differ in more than 25 amino acids from SEQ ID NO: 11 and is capable of binding TGFβ, wherein a difference can be a substitution, a deletion or an addition of an amino acid. In some embodiments, the TGFβ inhibitor has an amino acid sequence that is substantially identical to a sequence selected from the group consisting of SEQ ID NO: 11, SEQ ID NO: 12 and SEQ ID NO: 13. In some embodiments, the TGFβ inhibitor has an amino acid sequence that is substantially identical to SEQ ID NO: 11.

In some embodiments, the TGFβ inhibitor is a protein that is substantially identical, e.g., has at least 90% sequence identity, to the amino acid sequence of the TGFβR of bintrafusp alfa and is still capable of binding TGFβ. In some embodiments, the TGFβ inhibitor is a protein with an amino acid sequence with not more than 50, not more than 40, not more than 25, or not more than 10 amino acid residues different from the TGFβR of bintrafusp alfa that is still capable of binding TGFβ. In some embodiments, the TGFβ inhibitor has 100-160 amino acid residues or 110-140 amino acid residues. In some embodiments, the amino acid sequence of the TGFβ inhibitor is selected from the group consisting of a sequence corresponding to positions 1-136 of the TGFβR of bintrafusp alfa, a sequence corresponding to positions 20-136 of the TGFβR of bintrafusp alfa and a sequence corresponding to positions 22-136 of the TGFβR of bintrafusp alfa.

In some embodiments, the TGFβ inhibitor is selected from the group consisting of lerdelimumab, XPA681, XPA089, LY2382770, LY3022859, 1D11, 2G7, AP11014, A-80-01, LY364947, LY550410, LY580276, LY566578, SB-505124, SD-093, SD-208, SB-431542, ISTH0036, ISTH0047, galunisertib (LY2157299 monohydrate, a small molecule kinase inhibitor of TGF-β RI), LY3200882 (a small molecule kinase inhibitor TGF-β RI disclosed by Pei et al. (2017) CANCER RES 77(13 Suppl):Abstract 955), metelimumab (an antibody targeting TGF-β1, see Colak et al. (2017) TRENDS CANCER 3(1):56-71), fresolimumab (GC-1008; an antibody targeting TGF-β1 and TGF-β2), XOMA 089 (an antibody targeting TGF-β1 and TGF-β2; see Mirza et al. (2014) INVESTIGATIVE OPHTHALMOLOGY & VISUAL SCIENCE 55:1121), AVID200 (a TGF-β1 and TGF-β3 trap, see Thwaites et al. (2017) BLOOD 130:2532), Trabedersen/AP12009 (a TGF-β2 antisense oligonucleotide, see Jaschinski et al. (2011) CURR PHARM BIOTECHNOL. 12(12):2203-13), Belagen-pumatucel-L (a tumor cell vaccine targeting TGF-β2, see, e.g., Giaccone et al. (2015) EUR J CANCER 51(16):2321-9), TGB-β pathway targeting agents described in Colak et al. (2017), supra, including Ki26894, SD208, SM16, IMC-TR1, PF-03446962, TEW-7197, and GW788388.

Preferably, the PD-1 inhibitor and the TGFβ inhibitor are fused. In some embodiments, the fusion molecule is one of the PD-1 inhibitor and TGFβ inhibitor fusion proteins disclosed in WO 2015/118175, WO 2018/205985, WO 2020/014285 or WO 2020/006509. Preferably, the fusion molecule is an anti-PD-L1/TGFβ Trap molecule. Preferably, the N-terminal end of the sequence of the TGFβRII or the fragment thereof is fused to the C-terminal end of each heavy chain sequence of the antibody or fragment thereof. Preferably, the antibody or the fragment thereof and the extracellular domain of TGFβRII or the fragment thereof are genetically fused via a linker sequence. In some embodiments, the linker sequence is a short, flexible peptide. In a preferred embodiment, the linker sequence is (G₄S)ₓG, wherein x is 3-6, preferably 4-5, most preferably 4.

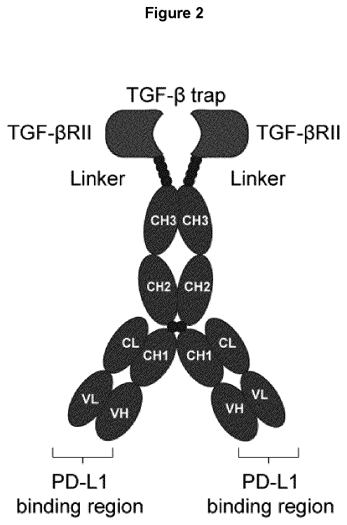

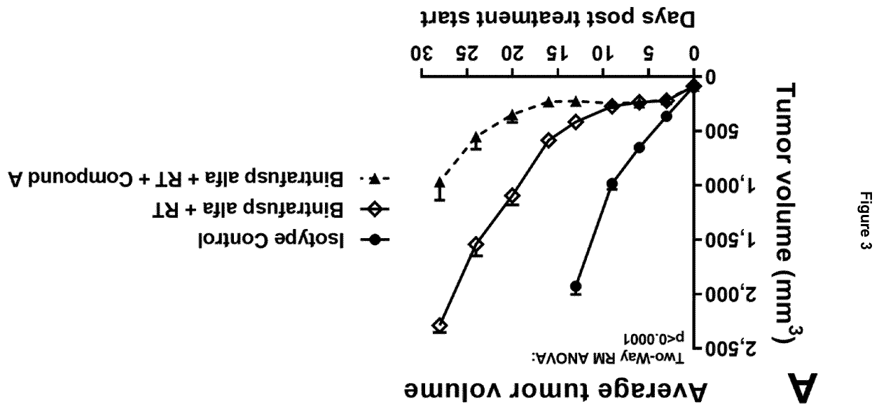

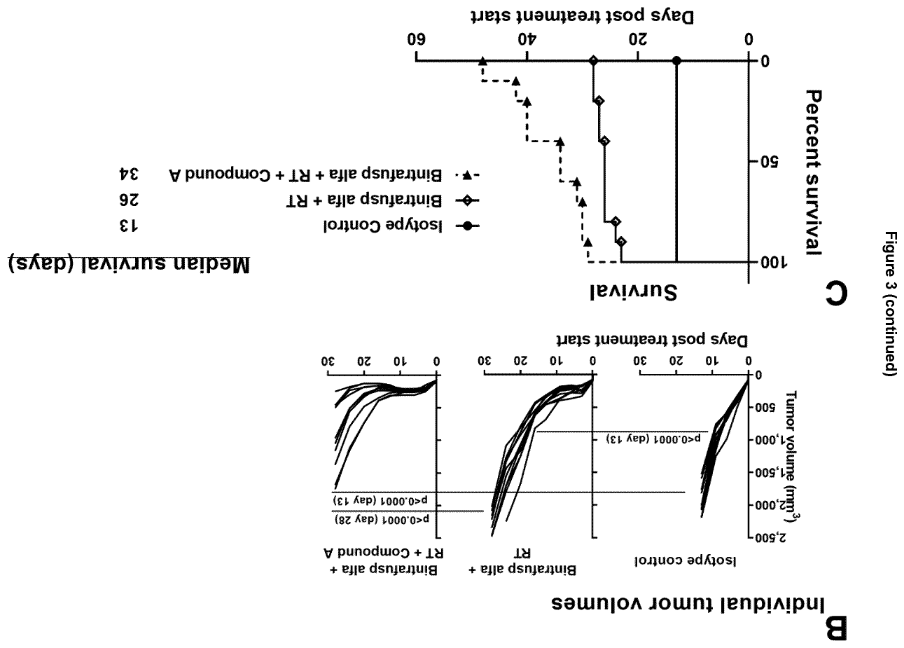

An exemplary anti-PD-L1/TGFβ Trap is shown in FIG. 2. The depicted heterotetramer consists of the two light chain sequences of the anti-PD-L1 antibody, and two sequences each comprising a heavy chain sequence of the anti-PD-L1 antibody which C-terminus is genetically fused via a linker sequence to the N-terminus of the extracellular domain of the TGFβRII or the fragment thereof.

In a preferred embodiment, the extracellular domain of TGFβRII or the fragment thereof of the anti-PD-L1/TGFβ Trap has an amino acid sequence that does not differ in more than 25 amino acids from SEQ ID NO: 11 and is capable of binding TGFβ, wherein a difference can be a substitution, a deletion or an addition of an amino acid. In some embodiments, the anti-PD-L1/TGFβ Trap is one of the anti-PD-L1/TGFβ Trap molecules disclosed in WO 2015/118175, WO 2018/205985. For instance, anti-PD-L1/TGFβ Trap may comprise the light chains and heavy chains of SEQ ID NO: 1 and SEQ ID NO: 3 of WO 2015/118175, respectively. In another embodiment, anti-PD-L1/TGFβ Trap is one of the constructs listed in Table 2 of WO 2018/205985, such as construct 9 or 15 thereof. In other embodiments, anti-PD-L1/TGFβ Trap is a heterotetramer, consisting of two light chain sequences each corresponding to SEQ ID NO: 12 of WO 2018/205985 and two sequences each comprising the heavy chain sequence corresponding to SEQ ID NO: 11 of WO 2018/205985 fused via a linker sequence (G₄S)ₓG to the TGFβRII extracellular domain sequence corresponding to SEQ ID NO: 14 (wherein “x” of the linker sequence is 4) or SEQ ID NO: 15 (wherein “x” of the linker sequence is 5) of WO 2018/205985. In another embodiment, the anti-PD-L1/TGFβ Trap is SHR1701. In a further embodiment, the PD-1 inhibitor and TGFβ inhibitor fusion protein is one of the fusion molecules disclosed in WO 2020/006509. In one embodiment, the PD-1 inhibitor and TGFβ inhibitor fusion protein is Bi-PLB-1, Bi-PLB-2 or Bi-PLB-1.2 disclosed in WO 2020/006509. In one embodiment, the PD-1 inhibitor and TGFβ inhibitor fusion protein is Bi-PLB-1.2 disclosed in WO 2020/006509. In one embodiment, the PD-1 inhibitor and TGFβ inhibitor fusion protein comprises SEQ ID NO:128 and SEQ ID NO:95 disclosed in WO 2020/006509. In some embodiments, the PD-1 inhibitor and TGFβ inhibitor fusion protein comprises heavy chain and light chain sequences respectively corresponding to the light chain sequences and the heavy chain sequences selected from the group consisting of: (1) SEQ ID NO: 7 and SEQ ID NO: 8 of the present disclosure, (2) SEQ ID NO: 15 and SEQ ID NO: 17 of the present disclosure, (3) SEQ ID NO: 15 and SEQ ID NO: 18 of the present disclosure and (4) SEQ ID NO:128 and SEQ ID NO:95 disclosed in WO 2020/006509. In some embodiments, the PD-1 inhibitor and TGFβ inhibitor fusion protein is still capable of binding PD-L1 and TGFβ and it comprises heavy chain and light chain sequences that are respectively substantially identical, e.g., have at least 90% sequence identity, to the light chain sequences and the heavy chain sequences selected from the group consisting of: (1) SEQ ID NO: 7 and SEQ ID NO: 8 of the present disclosure, (2) SEQ ID NO: 15 and SEQ ID NO: 17 of the present disclosure, (3) SEQ ID NO: 15 and SEQ ID NO: 18 of the present disclosure and (4) SEQ ID NO:128 and SEQ ID NO:95 disclosed in WO 2020/006509. In some embodiments, the amino acid sequence of the light chain sequences and the heavy chain sequences of the PD-1 inhibitor of the PD-1 inhibitor and TGFβ inhibitor fusion protein are respectively not more than 50, not more than 40, not more than 25, or not more than 10 amino acid residues different from the light chain sequences and the heavy chain sequences of the antibody moiety of bintrafusp alfa and the CDRs are fully identical with the CDRs of bintrafusp alfa and/or the PD-1 inhibitor is still capable of binding to PD-L1. In some embodiments, the amino acid sequence of the anti-PD-L1/TGFβ Trap is substantially identical, e.g., has at least 90% sequence identity, to the amino acid sequence of bintrafusp alfa and is capable of binding to PD-L1 and TGF-β.

In some embodiments, the amino acid sequence of the light chain sequences and the heavy chain sequences of the anti-PD-L1/TGFβ Trap are respectively selected from the group consisting of: (1) SEQ ID NO: 7 and SEQ ID NO: 8, (2) SEQ ID NO: 15 and SEQ ID NO: 17, and (3) SEQ ID NO: 15 and SEQ ID NO: 18. Preferably, the amino acid sequence of anti-PD-L1/TGFβ Trap is identical to the amino acid sequence of bintrafusp alfa. In some embodiments, the anti-PD-L1/TGFβ Trap is bintrafusp alfa.

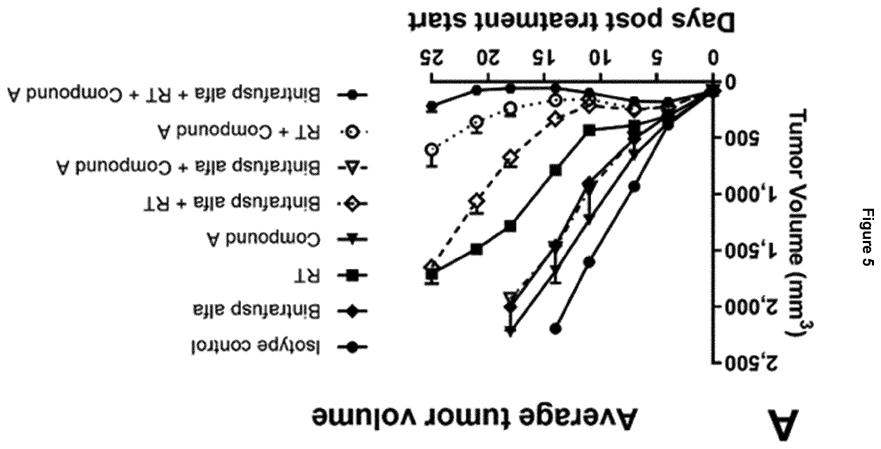

In a particular embodiment, the PD-1 inhibitor and TGFβ inhibitor fusion protein is one of the fusion molecules disclosed in WO 2020/014285 that binds both PD-1 and TGF-β, e.g. as depicted in FIG. 4 therein or as described in Example 1, including those identified in Tables 2-9, as specified in table 16, therein, and in particular a fusion protein that binds both PD-1 and TGF-β and comprising a sequence that is substantially identical, e.g., has at least 90% sequence identity, to SEQ ID NO:15 or SEQ ID NO:296 and a sequence that is substantially identical, e.g., has at least 90% sequence identity, to SEQ ID NO:16, SEQ ID NO:143, SEQ ID NO:144, SEQ ID NO:145, SEQ ID NO:294 or SEQ ID NO:295 therein. In an embodiment, the PD-1 inhibitor and TGFβ inhibitor fusion protein comprises SEQ ID NO:15 and SEQ ID NO:16 of WO 2020/014285. In an embodiment, the PD-1 inhibitor and TGFβ inhibitor fusion protein comprises SEQ ID NO:15 and SEQ ID NO:143 of WO 2020/014285. In an embodiment, the PD-1 inhibitor and TGFβ inhibitor fusion protein comprises SEQ ID NO:15 and SEQ ID NO:144 of WO 2020/014285. In an embodiment, the PD-1 inhibitor and TGFβ inhibitor fusion protein comprises SEQ ID NO:15 and SEQ ID NO:145 of WO 2020/014285. In an embodiment, the PD-1 inhibitor and TGFβ inhibitor fusion protein comprises SEQ ID NO:15 and SEQ ID NO:294 of WO 2020/014285. In an embodiment, the PD-1 inhibitor and TGFβ inhibitor fusion protein comprises SEQ ID NO:15 and SEQ ID NO:295 of WO 2020/014285. In an embodiment, the PD-1 inhibitor and TGFβ inhibitor fusion protein comprises SEQ ID NO:296 and SEQ ID NO:16 of WO 2020/014285. In an embodiment, the PD-1 inhibitor and TGFβ inhibitor fusion protein comprises SEQ ID NO:296 and SEQ ID NO:143 of WO 2020/014285. In an embodiment, the PD-1 inhibitor and TGFβ inhibitor fusion protein comprises SEQ ID NO:296 and SEQ ID NO:144 of WO 2020/014285. In an embodiment, the PD-1 inhibitor and TGFβ inhibitor fusion protein comprises SEQ ID NO:296 and SEQ ID NO:145 of WO 2020/014285. In an embodiment, the PD-1 inhibitor and TGFβ inhibitor fusion protein comprises SEQ ID NO:296 and SEQ ID NO:294 of WO 2020/014285. In an embodiment, the PD-1 inhibitor and TGFβ inhibitor fusion protein comprises SEQ ID NO:296 and SEQ ID NO:295 of WO 2020/014285. In a further embodiment, the PD-1 inhibitor and TGFβ inhibitor fusion protein is one of the fusion molecules disclosed in WO 2020/006509. In one embodiment, the PD-1 inhibitor and TGFβ inhibitor fusion protein is Bi-PB-1, Bi-PB-2 or Bi-PB-1.2 disclosed in WO 2020/006509. In one embodiment, the PD-1 inhibitor and TGFβ inhibitor fusion protein is Bi-PB-1.2 disclosed in WO 2020/006509. In one embodiment, the PD-1 inhibitor and TGFβ inhibitor fusion protein comprises SEQ ID NO:108 and SEQ ID NO:93 disclosed in WO 2020/006509.

Preferably, the ATM inhibitor inhibits the ATM kinase and has an IC₅₀ below 1 mM, more preferably below 100 μM, more preferably below 1 μM, more preferably below 100 nM and most preferably below 10 nM. Preferably, the ATM inhibitor possesses a specificity for inhibiting ATM over other kinases, preferably ATR, of at least 10-fold, more preferably at least 100-fold, most preferably at least 1000-fold, as measured by the ratio of IC₅₀ for ATM over IC₅₀ for other kinases.

Assays for measuring the IC₅₀ of an ATM inhibitor are well known to the person skilled in the art. The IC₅₀ may, for instance, be determined with the aid of the following biochemical ATM kinase assay. The assay consists of two steps: the enzymatic reaction and the detection step. Firstly, ATM protein and the test substance are incubated at different concentrations with addition of substrate protein p53 and ATP. ATM mediates the phosphorylation of p53 at several positions, including at amino acid S15. The amount of phosphorylated p53 is determined with the aid of specific antibodies and the TR-FRET technique. The enzymatic ATM assay is carried out as TR-FRET (HTRFTM, Cisbio Bioassays) based 384-well assay. In the first step, purified human recombinant ATM (human ATM, full length, GenBank ID NM_000051, expressed in a mammal cell line) is incubated in assay buffer for 15 minutes with the ATM inhibitor in various concentrations and without test substance as negative or neutral control. The assay buffer comprises 25 mM HEPES pH 8.0, 10 mM Mg(CH₃COO)₂, 1 mM MnCl₂, 0,1% BSA and 0,01% Brij 35, 5 mM dithiothreitol (DTT). The test-substance solutions are dispensed into the microtitre plates using an ECHO 555 (Labcyte). In the second step, purified human recombinant cmyc-labelled p53 (human p53, full length, GenBank ID BC003596, expressed in Sf21 insect cells) and ATP are added, and the reaction mixture is incubated at 22° C. for 30-35 minutes. The pharmacologically relevant assay volume is 5 μl. The final concentrations in the assay during incubation of the reaction mixture are 0.3-0.4 nM ATM, 50-75 nM p53 and 10 μM ATP. The enzymatic reaction is stopped by addition of EDTA. The formation of phosphorylated p53 as the result of the ATM-mediated reaction in the presence of ATP is detected via specific antibodies [labelled with the fluorophorene europium (Eu) as donor and d2 as acceptor (Cisbio Bioassays)] which enable FRET. 2 μl of antibody-containing stop solution (12.5 mM HEPES pH 8.0, 125 mM EDTA, 30 mM sodium chloride, 300 mM potassium fluoride, 0.1006% Tween-20, 0.005% Brij 35, 0.21 nM anti-phospho-p53(ser15)-Eu antibody and 15 nM anti-cmyc-d2 antibody) are added to the reaction mixture. After incubation, usually for 2 hours (between 1.5 and 15 h), for signal development, the plates are analysed in a plate reader (EnVision, PerkinElmer) using TRF mode (and with laser excitation). After excitation of the donor europium at a wavelength of 340 nm, the emitted fluorescence light both of the acceptor d2 at 665 nm and also of the donor Eu at 615 nm is measured. The amount of phosphorylated p53 is directly proportional to the quotient of the amounts of light emitted, i.e. the relative fluorescence units (RFU) at 665 nm and 615 nm. The measurement data are processed by means of Genedata Screener software. IC₅₀ determinations are carried out, in particular, by fitting a dose/action curve to the data points by means of nonlinear regression analysis.
IC₅₀=half-maximum inhibitory concentration
ATP=adenosine triphosphate
TR-FRET=time-resolved fluorescence resonance energy transfer
HTRF=homogeneous time resolved fluorescence
HEPES=2-(4-(2-hydroxyethyl)-1-piperazinyl)ethanesulfonic acid
Mg(CH₃COO)₂=magnesium acetate
MnCl₂=manganese(II) chloride
BSA=bovine serum albumin
EDTA=ethylenediamine tetraacetate
TRF=time resolved fluorescence

The ATM inhibitor may be selected from the following group:

| Compound | CAS-No.: | Chemical structure |
|---|---|---|
| KU-55933 | 587871-26-9 | 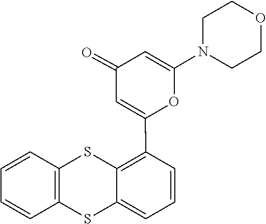 [SMILES: O=c1cc(-c2cccc3c2Sc2ccccc2S3)oc(N2CCOCC2)c1] |
| KU-60019 | 925701-46-8 | 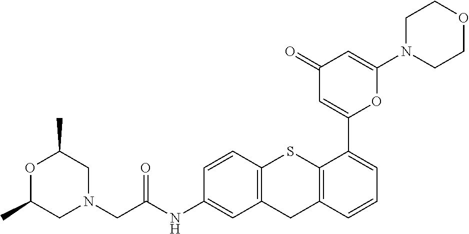 [SMILES: C[C@@H]1CN(CC(=O)Nc2ccc3c(c2)Cc2cccc(-c4cc(=O)cc(N5CCOCC5)o4)c2S3)C[C@H](C)O1] |
| Wortmannin | 19545-26-7 | 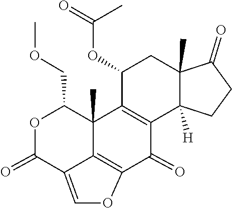 [SMILES: COC[C@H]1OC(=O)c2coc3c2[C@@]1(C)C1=C(C3=O)[C@@H]2CCC(=O)[C@@]2(C)C[C@H]1OC(C)=O] |
| CP-466722 | 1080622-86-1 | 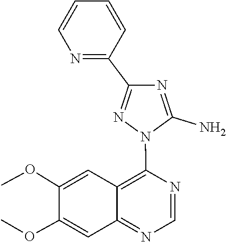 |
| Torin 2 | 1223001-51-1 | 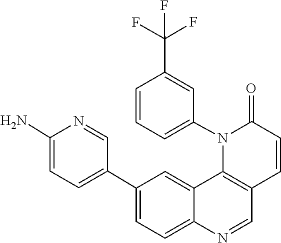 [SMILES: Nc1ccc(-c2ccc3ncc4ccc(=O)n(-c5cccc(C(F)(F)F)c5)c4c3c2)cn1] |
| CGK733 | 905973-89-9 | 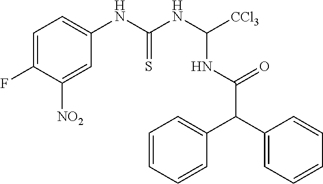 |
| ATM Inhibitor-1 | 2135639-94-8 | 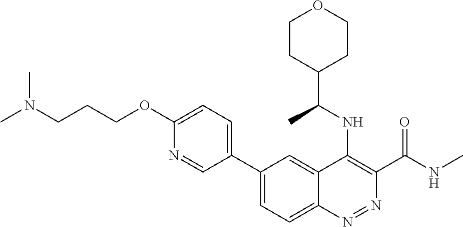 |
| AZD1390 | 2089288-03-7 | 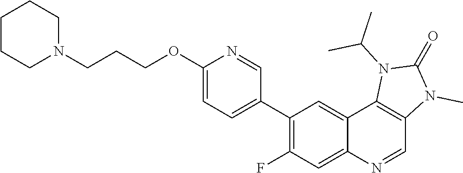 [SMILES: CC(C)n1c(=O)n(C)c2cnc3cc(F)c(-c4ccc(OCCCN5CCCCC5)nc4)cc3c21] |
| KU-59403 | 845932-30-1 | 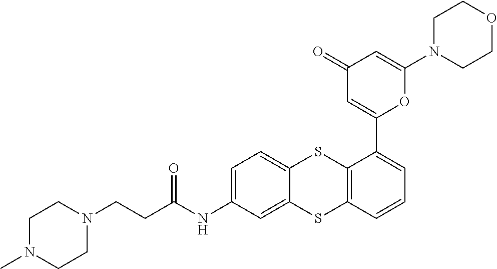 [SMILES: CN1CCN(CCC(=O)Nc2ccc3c(c2)Sc2cccc(-c4cc(=O)cc(N5CCOCC5)o4)c2S3)CC1] |
| AZD0156 | 1821428-35-6 | 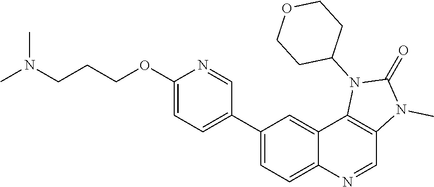 |

In some embodiments, the ATM inhibitor is an imidazo[4,5-c]quinoline derivative. In some embodiments, the ATM inhibitor is a compound of formula (I)

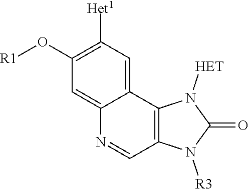

(I)
where
R1 denotes methyl,
R3 denotes methyl or H,
A in each case independently denotes unbranched or branched alkyl having 1, 2, 3, 4, 5, 6, 7, 8, 9 or 10 C atoms, where, independently of one another, 1, 2, 3, 4, 5, 6 or 7 H atoms may be replaced by Hal,
Het¹ is selected from the group consisting of pyridinyl, pyrimidinyl, pyrazolyl, triazolyl, imidazolyl, bezimidazolyl, imidazo[4,5-b]pyridinyl, and benzodiazolyl, each of which may be unsubstituted or mono-, di- or trisubstituted, independently of one another, by Hal, A, CN, —(CY₂)ₚ—OY, —(CY₂)ₚ—NYY, —(CY₂)ₚ—COOY, —(CY₂)ₚ—CO—NYY, —(CY₂)ₚ—NY—COY, —Het² and/or —SO₂—Het²,
Het² denotes a monocyclic saturated heterocycle having 2, 3, 4, 5, 6 or 7 C atoms and 1, 2, 3 or 4 N, O and/or S atoms, which may be unsubstituted or monosubstituted by A,
HET denotes a 5- or 6-membered aromatic heterocycle having 1, 2 or 3 N atoms and optionally an O atom or S atom, where this heterocycle is linked to the N atom of the skeleton via the ring C atom, and is selected from the group consisting of pyridinyl, pyrimidinyl, pyrazolyl, thiazolyl, imidazolyl; pyrrolo[3,2-c]pyridinyl, pyrrolo[2,3-b]pyridinyl and quinolinyl; and where this heterocycle may be unsubstituted or substituted by one, two or three substituents, which are selected, independently of one another, from the group consisting of: Hal, A, Het², CN, —(CY₂)ₚ—OY, —(CY₂)ₚ—OZ, —(CY₂)ₚ—O-Het², —(CY₂)ₚ—O—(CY₂)ₜ-Het², —(CY₂)ₚ—O—(CY₂)ₜ—NYY, —(CY₂)ₚ—O—(CY₂)ₜ—OY, —(CY₂)ₚ—O—(CY₂)ₜ—POAA, —(CY₂)ₚ—NYY, —(CY₂)ₚ—COOY, —(CY₂)ₚ—CO—NYY, —(CY₂)ₚ—NY—COY, —SO₂—Het², CyA, —(CY₂)ₚ—O—(CY₂)ₜ—SO₂—Y, —(CY₂)ₚ—NY—SO₂—Y, and —(CY₂)ₚ—SO₂—Y,
Y denotes H or A,
Z denotes unbranched or branched alkenyl having 2, 3, 4, 5, 6, 7, 8, 9 or 10 C atoms, where, independently of one another, 1, 2, 3, 4, 5, 6 or 7 H atoms may be replaced by Hal,
CyA denotes cycloalkyl having 3, 4, 5, 6, 7 or 8 ring C atoms which is unsubstituted or mono- or polysubstituted, independently of one another, by Hal, A, CN, —(CY₂)ₚ—OY, —(CY₂)ₚ—NYY, —(CY₂)ₚ—COOY, —(CY₂)ₚ—CO—NYY and/or —(CY₂)ₚ—NY—COY,
Hal denotes F, Cl, Br or I, and
p denotes 0, 1, 2, 3, 4, 5 or 6
t denotes 1, 2, 3, 4, 5 or 6,
and/or pharmaceutically acceptable salt thereof.

In certain exemplary embodiments, HET may be substituted by one, two, three or more substituents which are selected, independently of one another, from the group consisting of: F, Cl, methyl, ethyl, propyl, isopropyl, methoxy, ethoxy, propoxy, piperazinyl, tetrahydro-pyranyl, —CN, 2-methoxyethoxy, 2-hydroxyethoxy, fluoromethoxy, difluoromethoxy, N-methylcarbamoyl (—C(═O)—NH—CH₃), 2-methylaminoethoxy, 1-methyl-azetidin-3-ylmethoxy, trideuteriomethoxy, trifluoromethoxy, methylsulfonylmethoxy, methylsulfonyl, cyclopropyl, allyloxy, piperazinyl, and azetidinyloxy.

In some exemplary embodiments, HET is selected from the following 5- or 6-membered monocyclic aromatic heterocycles: pyridin-2-yl, pyridin-4-yl, 5-allyloxy-3-fluoropyridin-2-yl, 5-(azetidin-3-yloxy)-3-fluoropyridin-2-yl, 5-chloro-3-fluoropyridin-2-yl, 3-cyclopropylpyridin-4-yl, 3-cyclopropyl-5-fluoropyridin-4-yl, 3,5-difluoropyridin-2-yl, 3,5-difluoropyridin-4-yl, 5-difluoro-methoxy-3-fluoropyridin-2-yl, 3-difluoromethoxy-5-fluoropyridin-4-yl, 5-ethoxy-3-fluoropyridin-2-yl, 3-fluoro-5-(1-methylazetidin-3-ylmethoxy)pyridin-2-yl, 3-fluoro-5-methoxypyridin-4-yl, 3-fluoro-5-methoxypyridin-2-yl, 3-fluoro-5-fluoromethoxypyridin-2-yl, 3-fluoro-5-fluoromethoxypyridin-4-yl, 3-fluoropyridin-2-yl, 3-fluoro-5-methylsulfonylmethoxypyridin-2-yl, 3-fluoro-5-methylsulfonylpyridin-2-yl 3-fluoro-5-(2-methylamino-ethoxy)pyridin-2-yl, 3-fluoro-5-methylpyridin-4-yl, 3-fluoro-5-methylpyridin-2-yl, 3-fluoropyridin-4-yl, 3-fluoropyridin-2-yl, 3-fluoro-5-piperazin-1-ylpyridin-2-yl, 3-chloro-pyridin-4-yl, 3-ethylpyridin-4-yl, 3-ethyl-5-fluoropyridin-4-yl, 3-ethyl-5-methylpyridin-4-yl, 5-fluoropyridin-2-yl, 3-methylpyridin-4-yl, 3-methoxypyridin-4-yl, 2-cyano-pyridin-4-yl, 3-cyanopyridin-4-yl, 3-cyanopyridin-6-yl, 3-cyano-5-fluoropyridin-4-yl, 3-fluoro-5-(2-methoxyethoxy)pyridin-2-yl, 3-fluoro-5-(2-hydroxyethoxy)pyridin-2-yl, 3-fluoro-5-(trideuteriomethoxy)pyridin-4-yl, 3-fluoro-5-(trideuteriomethoxy)pyridin-2-yl, 5-fluoro-3-(N-methylcarbamoyl)pyridin-6-yl, 5-fluoropyrimidin-2-yl, 5-fluoropyrimidin-4-yl, pyrimidin-2-yl, pyrimidin-5-yl, 1 H-pyrazol-4-yl, 1-ethyl-3-methyl-1 H-pyrazol-4-yl, 1,2-dimethyl-1 H-pyrazol-4-yl, 1,3-dimethyl-1H-pyrazol-4-yl, 1-methyl-1H-pyrazol-4-yl, 1-(tetrahydropyran-4-yl)-1 H-pyrazol-4-yl, 2-methyl-2H-pyrazol-3-yl, 3-methyl-1H-pyrazol-4-yl, 2-methylthiazol-4-yl, 3,5-dimethyl-1 H-pyrazol-4-yl, 3-fluoro-1-methylpyrazol-4-yl, thiazol-2-yl, and 1-methyl-1H-imidazolyl.

In exemplary embodiments, Het¹ is unsubstituted or substituted by one or two substituents selected, independently of one another, from A, which may be unsubstituted or mono- or polysubstituted by Hal, in particular F, or from —OY, —NYY, Hal, and -Het², particularly preferably methyl, ethyl, amino, methoxy, fluoromethyl, difluoromethyl, fluorine, azetidinyl.

For instance, Het¹ may be selected from: 1H-pyrazol-4-yl, 2H-pyrazol-3-yl, 1H-pyrazol-3-yl, 1-methyl-1H-pyrazol-4-yl, 3-methyl-1H-pyrazol-4-yl, 5-methyl-1H-pyrazol-3-yl, 4-methyl-1H-pyrazol-3-yl, 1-fluoromethyl-1H-pyrazol-4-yl, 1-difluoromethyl-1 H-pyrazol-4-yl, 1,3-dimethyl-1 H-pyrazol-4-yl, 1-ethyl-1H-pyrazol-4-yl, 1-ethyl-3-methyl-1H-pyrazolyl, 3-fluoro-1-methyl-1H-pyrazol-4-yl, 3-amino-1H-pyrazol-5-yl, 2H-1,2,3-triazol-4-yl, 3H-1,2,3-triazol-4-yl-, 1-methyl-1H-1,2,3-triazol-4-yl, 2-methyl-2H-1,2,3-triazol-4-yl, 2-amino-1H-imidazol-4-yl, 6-methoxypyridin-3-yl, 1-(azetidin-3-yl)-3-methyl-1H-pyrazol-4-yl, 2-methyl-3H-benzimidazol-5-yl, and 2-methyl-1 H-imidazo[4,5-b]pyridin-6-yl.

In a further exemplary embodiment, the ATM inhibitor is a compound of formula (II)

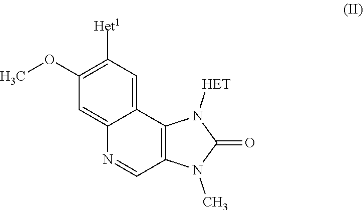

where
Het¹ is pyrazolyl, which may be unsubstituted or mono-, di- or trisubstituted, independently of one another, by Hal or A,
A in each case independently denotes unbranched or branched alkyl having 1, 2, 3, 4, 5 or 6 C atoms, where, independently of one another, 1, 2, 3, 4, or 5 H atoms may be replaced by Hal,
Hal denotes F, Cl, Br or I,
HET is pyridinyl, which is unsubstituted or substituted as described above for HET in formula (I), such as pyridin-4-yl, and may, for instance, be selected from 3-difluoromethoxy-5-fluoropyridin-4-yl, 3-fluoro-5-methoxypyridin-4-yl, 3-fluoro-5-fluoromethoxypyridin-4-yl, and 3-fluoro-5-(trideuteriomethoxy)pyridin-4-yl,
or pharmaceutically acceptable salt thereof.

In exemplary embodiments of a compound of Formula (II), Het¹ is selected from 1H-pyrazol-4-yl, 2H-pyrazol-3-yl, 1 H-pyrazol-3-yl, 1-methyl-1H-pyrazol-4-yl, 3-methyl-1H-pyrazol-4-yl, 5-methyl-1H-pyrazol-3-yl, 4-methyl-1H-pyrazol-3-yl, 1-fluoromethyl-1 H-pyrazol-4-yl, 1-difluoromethyl-1H-pyrazol-4-yl, 1,3-dimethyl-1 H-pyrazol-4-yl, 1-ethyl-1 H-pyrazol-4-yl, 1-ethyl-3-methyl-1H-pyrazolyl, and 3-fluoro-1-methyl-1 H-pyrazol-4-yl.

Preferably, the ATM inhibitor is any one selected from the group consisting of the compounds of claim 6 of WO 2012/028233 A1 or the compounds of claim 18 of WO 2016/155884 A1. More preferably, the ATM inhibitor is 3-Fluoro-4-[7-methoxy-3-methyl-8-(1-methyl-1 H-pyrazol-4-yl)-2-oxo-2,3-dihydroimidazo[4,5-c]-quinolin-1-yl]benzonitrile or 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one (also referred to as “Compound A”). Most preferably, the ATM inhibitor is Compound A. Compound A is described in detail in WO 2016/155884 A1 (designated as compound 4 in Table 2).

Surprisingly, it was discovered in due course that Compound A exists in the form of two atropisomers:

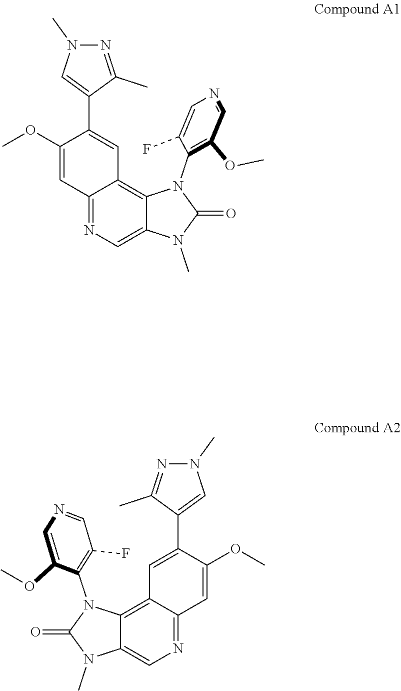

wherein the bold and dashed sections denote the partial rotation of the pyridine ring out of the plane in which the tricyclic ring is situated.

As apparent from the formulae above, Compound A1 is 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one and Compound A2 is 8-(1,3-Dimethyl-1H-pyrazol-4-yl)-1-(Ra)-(3-fluoro-5-methoxy-pyridin-4-yl)-7-methoxy-3-methyl-1,3-dihydro-imidazo[4,5-c]quinolin-2-one.

Compounds A1 and A2 can be obtained starting from Compound A using chromatography on a chiral stationary phase (see, e.g., Chiral Liquid Chromatography; W. J. Lough, Ed. Chapman and Hall, New York, (1989); Okamoto, “Optical resolution of dihydropyridine enantiomers by high-performance liquid chromatography using phenylcarbamates of polysaccharides as a chiral stationary phase”, J. of Chromatogr. 513:375-378, (1990)). Compounds 1 and 2 can be isolated by chromatography on chiral stationary phase, for example, a Chiralpak IC column (5 mm, 150×4.6 mm I.D.) e.g., using isocratic elution with a mobile phase containing: H₂O/ACN 50/50 v/v (using, for instance, a flow of 1.00 ml/min; UV measurement at 260 nm; T_{c} and Tₛ: 25±5° C., S_{conc} 0.20 mg/ml; injected volume 10 ml).

As an alternative to the above chromatographic method for separating Compound A into Compounds A1 and A2, preparative supercritical fluid chromatography may be used, involving for instance: Chiralpak AS-H (20 mm×250 mm, 5 μm) column; isocratic elution (20:80 ethanol:CO₂ with 0.1% v/v NH₃), BPR (back-pressure reg.): about 100 bar above atmospheric pressure; a column temperature of 40° C., a flow rate of 50 ml/min, an injection volume of 2500 μl (125 mg) and a detector wavelength of 265 nm.

In some embodiments, Compound A is Compound A2. In preferred embodiments, Compound A is Compound A1.

Unless otherwise specified, the terms “Compound A, A1 and A2” also include pharmaceutically acceptable salts thereof. It is understood that although the methods described herein may refer to formulations, doses and dosing regimens/schedules of Compound A, such formulations, doses and/or dosing regimens/schedules are equally applicable to any pharmaceutically acceptable salt of Compound A. Accordingly, in some embodiments, a dose or dosing regimen for a pharmaceutically acceptable salt of Compound A, or a pharmaceutically acceptable salt thereof, is selected from any of the doses or dosing regimens for Compound A as described herein. Possible dosages of Compound A are described in WO 2016/155884 A1. In an embodiment of the invention, Compound A, A1 or A2 is administered at a dose of 5 mg to 1 g per dosage unit, for instance between 10 and 750 mg per dosage unit, such as between 20 and 500 mg per dosage unit, such as 25, 50, 75, 100, 125, 150, 175, 200, 225, 250, 275, 300, 325 or 350 mg per unit. For instance, Compound A, A1 or A2 may be administered at dosages of about 1 to 750 mg per day, e.g. 20 to 500 mg per day, such as 25 to 400 mg day. A biologically efficacious dose for Compound A1 has been estimated to be in the range of 25 to 350 mg once daily.

3-Fluoro-4-[7-methoxy-3-methyl-8-(1-methyl-1 H-pyrazol-4-yl)-2-oxo-2,3-dihydroimidazo[4,5-c]-quinolin-1-yl]benzonitrile may be administered, for instance, at dosages of between 50 and 400 mg per day, such as 50, 100, 200, 300 or 400 mg/day.

A pharmaceutically acceptable salt may involve the inclusion of another molecule, such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In some embodiments, the radiotherapy is part of a chemoradiotherapy (CRT). The chemotherapeutic agent can be etoposide, doxorubicin, topotecan, irinotecan, fluorouracil, gemcitabine, paclitaxel, a platin, an anthracycline, and a combination thereof.

The radiotherapy can be a treatment given with electrons, photons, protons, alfa-emitters, other ions, radio-nucleotides, boron capture neutrons and combinations thereof. In some embodiments, the radiotherapy comprises about 35-70 Gy/20-35 fractions.

In one embodiment, the therapeutic combination of the invention is used in the treatment of a human subject. In one embodiment, the PD-1 inhibitor targets human PD-L1. The main expected benefit in the treatment with the therapeutic combination is a gain in risk/benefit ratio for these human patients.

In one embodiment, the cancer is identified as a PD-L1 positive cancerous disease. According to the invention, the cancer is preferably considered to be PD-L1 positive if between at least 0.1% and at least 10% of the cells of the cancer have PD-L1 present at their cell surface, more preferably between at least 0.5% and 5%, most preferably at least 1%. In one embodiment, the PD-L1 expression is determined by immunohistochemistry (IHC).

In certain embodiments, the invention provides for the treatment of diseases, disorders, and conditions characterized by excessive or abnormal cell proliferation. Such diseases include a proliferative or hyperproliferative disease. Examples of proliferative and hyperproliferative diseases include cancer and myeloproliferative disorders.

In another embodiment, the cancer is selected from carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, acute myeloid leukemia, multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma, cervical cancer, brain cancer, stomach cancer, bladder cancer, hepatoma, breast cancer, colon carcinoma, biliary tract cancer, and head and neck cancer. The disease or medical disorder in question may preferably be selected from any of those disclosed in WO2015118175, WO2018029367, WO2018208720, PCT/US18/12604, PCT/US19/47734, PCT/US19/40129, PCT/US19/36725, PCT/US19/732271, PCT/US19/38600, PCT/EP2019/061558.

In various embodiments, the method of the invention is employed as a first, second, third or later line of treatment. A line of treatment refers to a place in the order of treatment with different medications or other therapies received by a patient. First line therapy regimens are treatments given first, whereas second or third line therapy is given after the first line therapy or after the second line therapy, respectively. Therefore, first line therapy is the first treatment for a disease or condition. In patients with cancer, first line therapy, sometimes referred to as primary therapy or primary treatment, can be surgery, chemotherapy, radiation therapy, or a combination of these therapies. Typically, a patient is given a subsequent chemotherapy regimen (second or third line therapy), either because the patient did not show a positive clinical outcome or only showed a sub-clinical response to a first or second line therapy or showed a positive clinical response but later experienced a relapse, sometimes with disease now resistant to the earlier therapy that elicited the earlier positive response.

In some embodiments, the method of the invention is applied in a later line of treatment, particularly a second line or higher treatment of the cancer. There is no limitation to the prior number of therapies provided that the subject underwent at least one round of prior cancer therapy. The round of prior cancer therapy refers to a defined schedule/phase for treating a subject with, e.g., one or more chemotherapeutic agents, radiotherapy or chemoradiotherapy, and the subject failed with such previous treatment, which was either completed or terminated ahead of schedule. One reason could be that the cancer was resistant or became resistant to prior therapy. The current standard of care (SoC) for treating cancer patients often involves the administration of toxic and old chemotherapy regimens. Such SoC is associated with high risks of strong adverse events that are likely to interfere with the quality of life (such as secondary cancers). In one embodiment, the combined administration of the PD-1 inhibitor, TGFβ inhibitor, and ATM inhibitor may be as effective and better tolerated than the SoC in patients with cancer.

As the modes of action of the PD-1 inhibitor, TGFβ inhibitor, and ATM inhibitor are different, it is thought that the likelihood that administration of the therapeutic treatment of the invention may lead to enhanced immune-related adverse events (irAE) is small.

In one embodiment, the method of the invention is a second line or higher treatment of a cancer selected from the group of pre-treated relapsing metastatic NSCLC, unresectable locally advanced NSCLC, pre-treated SCLC ED, SCLC unsuitable for systemic treatment, pre-treated relapsing (recurrent) or metastatic SCCHN, recurrent SCCHN eligible for re-irradiation, and pre-treated microsatellite status instable low (MSI-L) or microsatellite status stable (MSS) metastatic colorectal cancer (mCRC). SCLC and SCCHN are particularly systemically pre-treated. MSI-L/MSS mCRC occurs in 85% of all mCRC.

In one embodiment, the cancer exhibits microsatellite instability (MSI). Microsatellite instability (“MSI”) is or comprises a change in the DNA of certain cells (such as tumor cells) in which the number of repeats of microsatellites (short, repeated sequences of DNA) is different from the number of repeats that was contained in the DNA from which it was inherited. Microsatellite instability arises from a failure to repair replication-associated errors due to a defective DNA mismatch repair (MMR) system. This failure allows persistence of mismatch mutations all over the genome, but especially in regions of repetitive DNA known as microsatellites, leading to increased mutational load. It has been demonstrated that at least some tumors characterized by a high microsatellite instability (MSI-H) status have improved responses to certain anti-PD-1 agents (Le et al. (2015) N. Engl. J. Med. 372(26):2509-2520; Westdorp et al. (2016) Cancer Immunol. Immunother. 65(10): 1249-1259).

In some embodiments, a cancer has a MSI-H status. In some embodiments, a cancer has a low microsatellite instability (MSI-L) status. In some embodiments, a cancer has a microsatellite stable (MSS) status. In some embodiments microsatellite instability status is assessed by a next generation sequencing (NGS)-based assay, an immunohistochemistry (IHC)-based assay, and/or a PCR-based assay. In some embodiments, microsatellite instability is detected by NGS. In some embodiments, microsatellite instability is detected by IHC. In some embodiments, microsatellite instability is detected by PCR.

In some embodiments, the cancer is associated with a high tumor mutation burden (TMB). In some embodiments, the cancer is associated with high TMB and MSI-H. In some embodiments, the cancer is associated with high TMB and MSI-L or MSS. In some embodiments, the cancer is endometrial cancer associated with high TMB. In some related embodiments, the endometrial cancer is associated with high TMB and MSI-H. In some related embodiments, the endometrial cancer is associated with high TMB and MSI-L or MSS.

In some embodiments, a cancer is a mismatch repair deficient (dMMR) cancer.

In some embodiments, a cancer is a hypermutated cancer. In some embodiments, a cancer harbors a mutation in polymerase epsilon (POLE). In some embodiments, a cancer harbors a mutation in polymerase delta (POLD).

In some embodiments, a cancer is endometrial cancer (e.g. MSI-H or MSS/MSI-L endometrial cancer). In some embodiments, a cancer is a MSI-H cancer comprising a mutation in POLE or POLD (e.g. a MSI-H non-endometrial cancer comprising a mutation in POLE or POLD).

In some embodiments, the cancer is an advanced cancer. In some embodiments, the cancer is a metastatic cancer. In some embodiments, the cancer is a recurrent cancer (e.g. a recurrent gynecological cancer such as recurrent epithelial ovarian cancer, recurrent fallopian tube cancer, recurrent primary peritoneal cancer, or recurrent endometrial cancer). In one embodiment, the cancer is recurrent or advanced.

In one embodiment, the cancer is selected from: appendiceal cancer, bladder cancer, breast cancer, cervical cancer, colorectal cancer, endometrial cancer, esophageal cancer (in particular esophageal squamous cell carcinoma), fallopian tube cancer, gastric cancer, glioma (such as diffuse intrinsic pontine glioma), head and neck cancer (in particular head and neck squamous cell carcinoma and oropharyngeal cancer), leukemia (in particular acute lymphoblastic leukemia, acute myeloid leukemia) lung cancer (in particular non-small cell lung cancer (NSCLC)), lymphoma (in particular Hodgkin's lymphoma, non-Hodgkin's lymphoma), melanoma, mesothelioma (in particular malignant pleural mesothelioma), Merkel cell carcinoma, neuroblastoma, oral cancer, osteosarcoma, ovarian cancer, prostate cancer, renal cancer, salivary gland tumor, sarcoma (in particular Ewing's sarcoma or rhabdomyosarcoma) squamous cell carcinoma, soft tissue sarcoma, thymoma, thyroid cancer, urothelial cancer, uterine cancer, vaginal cancer, vulvar cancer or Wilms tumor. In a further embodiment, the cancer is selected from: appendiceal cancer, bladder cancer, cervical cancer, colorectal cancer, esophageal cancer, head and neck cancer, melanoma, mesothelioma, NSCLC, prostate cancer and urothelial cancer. In a further embodiment, the cancer is selected from cervical cancer, endometrial cancer, head and neck cancer (in particular head and neck squamous cell carcinoma and oropharyngeal cancer), lung cancer (in particular NSCLC), lymphoma (in particular non-Hodgkin's lymphoma), melanoma, oral cancer, thyroid cancer, urothelial cancer or uterine cancer. In another embodiment, the cancer is selected from head and neck cancer (in particular head and neck squamous cell carcinoma and oropharyngeal cancer), lung cancer (in particular NSCLC), urothelial cancer, melanoma or cervical cancer.

In one embodiment, the human has a solid tumor. In one embodiment, the solid tumor is an advanced solid tumor. In one embodiment, the cancer is selected from head and neck cancer, squamous cell carcinoma of the head and neck (SCCHN or HNSCC), gastric cancer, melanoma, renal cell carcinoma (RCC), esophageal cancer, non-small cell lung carcinoma, prostate cancer, colorectal cancer, ovarian cancer and pancreatic cancer. In one embodiment, the cancer is selected from the group consisting of: colorectal cancer, cervical cancer, bladder cancer, urothelial cancer, head and neck cancer, melanoma, mesothelioma, non-small cell lung carcinoma, prostate cancer, esophageal cancer, and esophageal squamous cell carcinoma. In one aspect the human has one or more of the following: SCCHN, colorectal cancer, esophageal cancer, cervical cancer, bladder cancer, breast cancer, head and neck cancer, ovarian cancer, melanoma, renal cell carcinoma (RCC), esophageal squamous cell carcinoma, non-small cell lung carcinoma, mesothelioma (e.g. pleural malignant mesothelioma), and prostate cancer.

In another aspect the human has a liquid tumor such as diffuse large B cell lymphoma (DLBCL), multiple myeloma, chronic lymphoblastic leukemia, follicular lymphoma, acute myeloid leukemia and chronic myelogenous leukemia.

In one embodiment, the cancer is head and neck cancer. In one embodiment, the cancer is HNSCC. Squamous cell carcinoma is a cancer that arises from particular cells called squamous cells. Squamous cells are found in the outer layer of skin and in the mucous membranes, which are the moist tissues that line body cavities such as the airways and intestines. Head and neck squamous cell carcinoma (HNSCC) develops in the mucous membranes of the mouth, nose, and throat. HNSCC is also known as SCCHN and squamous cell carcinoma of the head and neck.

HNSCC can occur in the mouth (oral cavity), the middle part of the throat near the mouth (oropharynx), the space behind the nose (nasal cavity and paranasal sinuses), the upper part of the throat near the nasal cavity (nasopharynx), the voicebox (larynx), or the lower part of the throat near the larynx (hypopharynx). Depending on the location, the cancer can cause abnormal patches or open sores (ulcers) in the mouth and throat, unusual bleeding or pain in the mouth, sinus congestion that does not clear, sore throat, earache, pain when swallowing or difficulty swallowing, a hoarse voice, difficulty breathing, or enlarged lymph nodes. HNSCC can metastasize to other parts of the body, such as the lymph nodes, lungs or liver. Tobacco use and alcohol consumption are the two most important risk factors for the development of HNSCC, and their contributions to risk are synergistic. In addition, the human papillomavirus (HPV), especially HPV-16, is now a well-established independent risk factor. Patients with HNSCC have a relatively poor prognosis. Recurrent/metastatic (R/M) HNSCC is especially challenging, regardless of human papillomavirus (HPV) status, and currently, few effective treatment options are available in the art. HPV-negative HNSCC is associated with a locoregional relapse rate of 19-35% and a distant metastatic rate of 14-22% following standard of care, compared with rates of 9-18% and 5-12%, respectively, for HPV-positive HNSCC. The median overall survival for patients with R/M disease is 10-13 months in the setting of first line chemotherapy and 6 months in the second line setting. The current standard of care is platinum-based doublet chemotherapy with or without cetuximab. Second line standard of care options include cetuximab, methotrexate, and taxanes. All of these chemotherapeutic agents are associated with significant side effects, and only 10-13% of patients respond to treatment. HNSCC regressions from existing systemic therapies are transient and do not add significantly increased longevity, and virtually all patients succumb to their malignancy.

In one embodiment, the cancer is head and neck cancer. In one embodiment the cancer is head and neck squamous cell carcinoma (HNSCC). In one embodiment, the cancer is recurrent/metastatic (R/M) HNSCC. In one embodiment, the cancer is recurring/refractory (R/R) HNSCC. In one embodiment, the cancer is HPV-negative or HPV-positive HNSCC. In one embodiment, the cancer is a locally advanced HNSCC. In one embodiment, the cancer is HNSCC, such as (R/M) HNSCC, in PD-L1 positive patients having a CPS of 1% or a TPS≥50%. The CPS or TPS is as determined by an FDA- or EMA-approved test, such as the Dako IHC 22C3 PharmDx assay. In one embodiment, the cancer is HNSCC in PD-1 inhibitor experienced or PD-1 inhibitor naïve patients. In one embodiment, the cancer is HNSCC in PD-1 inhibitor experienced or PD-1 inhibitor naïve patients.

In one embodiment, the head and neck cancer is oropharyngeal cancer. In one embodiment, the head and neck cancer is an oral cancer (i.e. a mouth cancer).

In one embodiment, the cancer is lung cancer. In some embodiments, the lung cancer is a squamous cell carcinoma of the lung. In some embodiments, the lung cancer is small cell lung cancer (SCLC). In some embodiments, the lung cancer is non-small cell lung cancer (NSCLC), such as squamous NSCLC. In some embodiments, the lung cancer is an ALK-translocated lung cancer (e.g. ALK-translocated NSCLC). In some embodiments, the cancer is NSCLC with an identified ALK translocation. In some embodiments, the lung cancer is an EGFR-mutant lung cancer (e.g. EGFR-mutant NSCLC). In some embodiments, the cancer is NSCLC with an identified EGFR mutation. In one embodiment, the cancer is NSCLC in PD-L1 positive patients having a TPS 1% or a TPS≥50%. The TPS is as determined by an FDA- or EMA-approved test, such as the Dako IHC 22C3 PharmDx assay or the VENTANA PD-L1 (SP263) assay. In one embodiment, the cancer is melanoma. In some embodiments, the melanoma is an advanced melanoma. In some embodiments, the melanoma is a metastatic melanoma. In some embodiments, the melanoma is a MSI-H melanoma. In some embodiments, the melanoma is a MSS melanoma. In some embodiments, the melanoma is a POLE-mutant melanoma. In some embodiments, the melanoma is a POLD-mutant melanoma. In some embodiments, the melanoma is a high TMB melanoma.

In one embodiment, the cancer is colorectal cancer. In some embodiments, the colorectal cancer is an advanced colorectal cancer. In some embodiments, the colorectal cancer is a metastatic colorectal cancer. In some embodiments, the colorectal cancer is a MSI-H colorectal cancer. In some embodiments, the colorectal cancer is a MSS colorectal cancer. In some embodiments, the colorectal cancer is a POLE-mutant colorectal cancer. In some embodiments, the colorectal cancer is a POLD-mutant colorectal cancer. In some embodiments, the colorectal cancer is a high TMB colorectal cancer.

In some embodiments, the cancer is a gynecologic cancer (i.e. a cancer of the female reproductive system such as ovarian cancer, fallopian tube cancer, cervical cancer, vaginal cancer, vulvar cancer, uterine cancer, or primary peritoneal cancer, or breast cancer). In some embodiments, cancers of the female reproductive system include, but are not limited to, ovarian cancer, cancer of the fallopian tube(s), peritoneal cancer, and breast cancer.

In some embodiments, the cancer is ovarian cancer (e.g. serous or clear cell ovarian cancer). In some embodiments, the cancer is fallopian tube cancer (e.g. serous or clear cell fallopian tube cancer). In some embodiments, the cancer is primary peritoneal cancer (e.g. serous or clear cell primary peritoneal cancer).

In some embodiments, the ovarian cancer is an epithelial carcinoma. Epithelial carcinomas make up 85% to 90% of ovarian cancers. While historically considered to start on the surface of the ovary, new evidence suggests at least some ovarian cancer begins in special cells in a part of the fallopian tube. The fallopian tubes are small ducts that link a woman's ovaries to her uterus that are a part of a woman's reproductive system. In a normal female reproductive system, there are two fallopian tubes, one located on each side of the uterus. Cancer cells that begin in the fallopian tube may go to the surface of the ovary early on. The term “ovarian cancer” is often used to describe epithelial cancers that begin in the ovary, in the fallopian tube, and from the lining of the abdominal cavity, call the peritoneum. In some embodiments, the cancer is or comprises a germ cell tumor. Germ cell tumors are a type of ovarian cancer that develops in the egg-producing cells of the ovaries. In some embodiments, a cancer is or comprises a stromal tumor. Stromal tumors develop in the connective tissue cells that hold the ovaries together, which sometimes is the tissue that makes female hormones called estrogen.

In some embodiments, the cancer is or comprises a granulosa cell tumor. Granulosa cell tumors may secrete estrogen resulting in unusual vaginal bleeding at the time of diagnosis. In some embodiments, a gynecologic cancer is associated with homologous recombination repair deficiency/homologous repair deficiency (HRD) and/or BRCA1/2 mutation(s). In some embodiments, a gynecologic cancer is platinum-sensitive. In some embodiments, a gynecologic cancer has responded to a platinum-based therapy. In some embodiments, a gynecologic cancer has developed resistance to a platinum-based therapy. In some embodiments, a gynecologic cancer has at one time shown a partial or complete response to platinum-based therapy (e.g. a partial or complete response to the last platinum-based therapy or to the penultimate platinum-based therapy). In some embodiments, a gynecologic cancer is now resistant to platinum-based therapy.

In some embodiments, the cancer is breast cancer. Usually breast cancer either begins in the cells of the milk producing glands, known as the lobules, or in the ducts. Less commonly breast cancer can begin in the stromal tissues. These include the fatty and fibrous connective tissues of the breast. Over time the breast cancer cells can invade nearby tissues such the underarm lymph nodes or the lungs in a process known as metastasis. The stage of a breast cancer, the size of the tumor and its rate of growth are all factors which determine the type of treatment that is offered. Treatment options include surgery to remove the tumor, drug treatment which includes chemotherapy and hormonal therapy, radiation therapy and immunotherapy. The prognosis and survival rate varies widely; the five year relative survival rates vary from 98% to 23% depending on the type of breast cancer that occurs. Breast cancer is the second most common cancer in the world with approximately 1.7 million new cases in 2012 and the fifth most common cause of death from cancer, with approximately 521,000 deaths. Of these cases, approximately 15% are triple-negative, which do not express the estrogen receptor, progesterone receptor (PR) or HER2. In some embodiments, triple negative breast cancer (TNBC) is characterized as breast cancer cells that are estrogen receptor expression negative (<1% of cells), progesterone receptor expression negative (<1% of cells), and HER2-negative. In one embodiment, the cancer is TNBC in PD-L1 positive patients having PD-L1 expressing tumor-infiltrating immune cells (IC) of ≥1%. The IC is as determined by an FDA- or EMA-approved test, such as the Ventana PD-L1 (SP142) assay.

In some embodiments, the cancer is estrogen receptor (ER)-positive breast cancer, ER-negative breast cancer, PR-positive breast cancer, PR-negative breast cancer, HER2-positive breast cancer, HER2-negative breast cancer, BRCA1/2-positive breast cancer, BRCA1/2-negative cancer, or TNBC. In some embodiments, the breast cancer is a metastatic breast cancer. In some embodiments, the breast cancer is an advanced breast cancer. In some embodiments, the cancer is a stage II, stage Ill or stage IV breast cancer. In some embodiments, the cancer is a stage IV breast cancer. In some embodiments, the breast cancer is a triple negative breast cancer.

In one embodiment, the cancer is endometrial cancer. Endometrial carcinoma is the most common cancer of the female genital, tract accounting for 10-20 per 100,000 person-years.

The annual number of new cases of endometrial cancer (EC) is estimated at about 325 thousand worldwide. Further, EC is the most commonly occurring cancer in post-menopausal women. About 53% of endometrial cancer cases occur in developed countries. In 2015, approximately 55,000 cases of EC were diagnosed in the U.S. and no targeted therapies are currently approved for use in EC. There is a need for agents and regimens that improve survival for advanced and recurrent EC in 1 L and 2 L settings. Approximately 10,170 people were predicted to die from EC in the U.S. in 2016. The most common histologic form is endometrioid adenocarcinoma, representing about 75-80% of diagnosed cases. Other histologic forms include uterine papillary serous (less than 10%), clear cell 4%, mucinous 1%, squamous less than 1% and mixed about 10%.

From the pathogenetic point of view, EC falls into two different types, so-called types I and II. Type I tumors are low-grade and estrogen-related endometrioid carcinomas (EEC), while type II are non-endometrioid (NEEC) (mainly serous and clear cell) carcinomas. The World Health Organization has updated the pathologic classification of EC, recognizing nine different subtypes of EC, but EEC and serous carcinoma (SC) account for the vast majority of cases. EECs are estrogen-related carcinomas, which occur in perimenopausal patients, and are preceded by precursor lesions (endometrial hyperplasia/endometrioid intraepithelial neoplasia). Microscopically, low-grade EEC (EEC 1-2) contains tubular glands, somewhat resembling the proliferative endometrium, with architectural complexity with fusion of the glands and cribriform pattern. High-grade EEC shows solid pattern of growth. In contrast, SC occurs in postmenopausal patients in absence of hyperestrogenism. At the microscopic level, SC shows thick, fibrotic or edematous papillae with prominent stratification of tumor cells, cellular budding, and anaplastic cells with large, eosinophilic cytoplasms. The vast majority of EEC are low-grade tumors (grades 1 and 2), and are associated with good prognosis when they are restricted to the uterus. Grade 3 EEC (EEC3) is an aggressive tumor, with increased frequency of lymph node metastasis. SCs are very aggressive, unrelated to estrogen stimulation, mainly occurring in older women. EEC 3 and SC are considered high-grade tumors. SC and EEC3 have been compared using the surveillance, epidemiology and End Results (SEER) program data from 1988 to 2001. They represented 10% and 15% of EC respectively, but accounted for 39% and 27% of cancer death respectively. Endometrial cancers can also be classified into four molecular subgroups: (1) ultramutated/POLE-mutant; (2) hypermutated MSI+(e.g., MSI-H or MSI-L); (3) copy number low/micro satellite stable (MSS); and (4) copy number high/serous-like. Approximately 28% of cases are MSI-high. (Murali, Lancet Oncol. (2014). In some embodiments, the patient has a mismatch repair deficient subset of 2 L endometrial cancer. In some embodiments, the endometrial cancer is metastatic endometrial cancer. In some embodiments, the patient has a MSS endometrial cancer. In some embodiments, the patient has a MSI-H endometrial cancer.

In one embodiment, the cancer is cervical cancer. In some embodiments, the cervical cancer is an advanced cervical cancer. In some embodiments, the cervical cancer is a metastatic cervical cancer. In some embodiments, the cervical cancer is a MSI-H cervical cancer. In some embodiments, the cervical cancer is a MSS cervical cancer. In some embodiments, the cervical cancer is a POLE-mutant cervical cancer. In some embodiments, the cervical cancer is a POLD-mutant cervical cancer. In some embodiments, the cervical cancer is a high TMB cervical cancer. In one embodiment, the cancer is cervical cancer in PD-L1 positive patients having a CPS 1%. The CPS is as determined by an FDA- or EMA-approved test, such as the Dako IHC 22C3 PharmDx assay.

In one embodiment, the cancer is uterine cancer. In some embodiments, the uterine cancer is an advanced uterine cancer. In some embodiments, the uterine cancer is a metastatic uterine cancer. In some embodiments, the uterine cancer is a MSI-H uterine cancer. In some embodiments, the uterine cancer is a MSS uterine cancer. In some embodiments, the uterine cancer is a POLE-mutant uterine cancer. In some embodiments, the uterine cancer is a POLD-mutant uterine cancer. In some embodiments, the uterine cancer is a high TMB uterine cancer. In one embodiment, the cancer is urothelial cancer. In some embodiments, the urothelial cancer is an advanced urothelial cancer. In some embodiments, the urothelial cancer is a metastatic urothelial cancer. In some embodiments, the urothelial cancer is a MSI-H urothelial cancer. In some embodiments, the urothelial cancer is a MSS urothelial cancer. In some embodiments, the urothelial cancer is a POLE-mutant urothelial cancer. In some embodiments, the urothelial cancer is a POLD-mutant urothelial cancer. In some embodiments, the urothelial cancer is a high TMB urothelial cancer. In one embodiment, the cancer is urothelial carcinoma in PD-L1 positive patients having a CPS 10%. The CPS is as determined by an FDA- or EMA-approved test, such as the Dako IHC 22C3 PharmDx assay. In one embodiment, the cancer is urothelial carcinoma in PD-L1 positive patients having PD-L1 expressing tumor-infiltrating immune cells (IC) of ≥5%. The IC is as determined by an FDA- or EMA-approved test, such as the Ventana PD-L1 (SP142) assay.

In one embodiment, the cancer is thyroid cancer. In some embodiments, the thyroid cancer is an advanced thyroid cancer. In some embodiments, the thyroid cancer is a metastatic thyroid cancer. In some embodiments, the thyroid cancer is a MSI-H thyroid cancer. In some embodiments, the thyroid cancer is a MSS thyroid cancer. In some embodiments, the thyroid cancer is a POLE-mutant thyroid cancer. In some embodiments, the thyroid cancer is a POLD-mutant thyroid cancer. In some embodiments, the thyroid cancer is a high TMB thyroid cancer.

Tumors may be a hematopoietic (or hematologic or hematological or blood-related) cancer, for example, cancers derived from blood cells or immune cells, which may be referred to as “liquid tumors”. Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, monoclonal gammopathy of undetermined (or unknown or unclear) significance (MGUS) and Waldenstrom's macroglobulinemia; lymphomas such as non-Hodgkin's lymphoma, Hodgkin's lymphoma, and the like.

In one embodiment, the cancer is a gastric cancer (GC) or a gastroesophageal junction cancer (GEJ). In one embodiment, the cancer is GC or GEJ in PD-L1 positive patients having a CPS≥1%. The CPS is as determined by an FDA- or EMA-approved test, such as the Dako IHC 22C3 PharmDx assay.

In one embodiment, the cancer is esophageal squamous cell carcinoma (ESCC). In one embodiment, the cancer is ESCC in PD-L1 positive patients having a CPS≥10%. The CPS is as determined by an FDA- or EMA-approved test, such as the Dako IHC 22C3 PharmDx assay.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid or myelocytic) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

In one embodiment, the cancer is non-Hodgkin's lymphoma. Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B cell lymphomas include follicular lymphoma (FL); small lymphocytic lymphoma (SLL); marginal zone lymphoma (MZL), including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large B cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphomas (T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenström's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

In one embodiment, the treatment is first line or second line treatment of HNSCC. In one embodiment, the treatment is first line or second line treatment of recurrent/metastatic HNSCC.

In one embodiment the treatment is first line treatment of recurrent/metastatic (1 L R/M) HNSCC. In one embodiment, the treatment is first line treatment of 1 L R/M HNSCC that is PD-L1 positive. In one embodiment the treatment is second line treatment of recurrent/metastatic (2 L R/M) HNSCC.

In one embodiment, the treatment is first line, second line, third line, fourth line or fifth line treatment of PD-1/PD-L1-naïve HNSCC. In one embodiment, the treatment first line, second line, third line, fourth line or fifth line treatment of PD-1/PD-L1 experienced HNSCC.

In some embodiments, the treatment of cancer is first line treatment of cancer. In one embodiment, the treatment of cancer is second line treatment of cancer. In some embodiments, the treatment is third line treatment of cancer. In some embodiments, the treatment is fourth line treatment of cancer. In some embodiments, the treatment is fifth line treatment of cancer. In some embodiments, prior treatment to said second line, third line, fourth line or fifth line treatment of cancer comprises one or more of radiotherapy, chemotherapy, surgery or radiochemotherapy.

In one embodiment, the prior treatment comprises treatment with diterpenoids, such as paclitaxel, nab-paclitaxel or docetaxel; vinca alkaloids, such as vinblastine, vincristine, or vinorelbine; platinum coordination complexes, such as cisplatin or carboplatin; nitrogen mustards such as cyclophosphamide, melphalan, or chlorambucil; alkyl sulfonates such as busulfan; nitrosoureas such as carmustine; triazenes such as dacarbazine; actinomycins such as dactinomycin; anthrocyclins such as daunorubicin or doxorubicin; bleomycins; epipodophyllotoxins such as etoposide or teniposide; antimetabolite anti-neoplastic agents such as fluorouracil, methotrexate, cytarabine, mecaptopurine, thioguanine, or gemcitabine; methotrexate; camptothecins such as irinotecan or topotecan; rituximab; ofatumumab; trastuzumab; cetuximab; bexarotene; sorafenib; erbB inhibitors such as lapatinib, erlotinib or gefitinib; pertuzumab; ipilimumab; nivolumab; FOLFOX; capecitabine; FOLFIRI; bevacizumab; atezolizumab; selicrelumab; obinotuzumab or any combinations thereof. In one embodiment, prior treatment to said second line treatment, third line, fourth line or fifth line treatment of cancer comprises ipilimumab and nivolumab. In one embodiment, prior treatment to said second line treatment, third line, fourth line or fifth line treatment of cancer comprises FOLFOX, capecitabine, FOLFIRI/bevacizumab and atezolizumab/selicrelumab. In one embodiment, prior treatment to said second line treatment, third line, fourth line or fifth line treatment of cancer comprises carboplatin/Nab-paclitaxel. In one embodiment, prior treatment to said second line treatment, third line, fourth line or fifth line treatment of cancer comprises nivolumab and electrochemotherapy. In one embodiment, prior treatment to said second line treatment, third line, fourth line or fifth line treatment of cancer comprises radiotherapy, cisplatin and carboplatin/paclitaxel.

In one embodiment, the treatment is first line or second line treatment of head and neck cancer (in particular head and neck squamous cell carcinoma and oropharyngeal cancer). In one embodiment, the treatment is first line or second line treatment of recurrent/metastatic HNSCC. In one embodiment the treatment is first line treatment of recurrent/metastatic (1 L R/M) HNSCC. In one embodiment, the treatment is first line treatment of 1 L R/M HNSCC that is PD-L1 positive. In one embodiment the treatment is second line treatment of recurrent/metastatic (2 L R/M) HNSCC.

In one embodiment, the treatment is first line, second line, third line, fourth line or fifth line treatment of PD-1/PD-L1-naïve HNSCC. In one embodiment, the treatment is first line, second line, third line, fourth line or fifth line treatment of PD-1/PD-L1 experienced HNSCC.

In some embodiments, the treatment results in one or more of increased tumor infiltrating lymphocytes including cytotoxic T cells, helper T cell and NK cells, increased T cells, increased granzyme B+ cells, reduced proliferating tumor cells and increased activated T cells as compared to levels prior to treatment (e.g. baseline level). Activated T cells may be observed by greater OX40 and human leukocyte antigen DR expression. In some embodiments, treatment results in upregulation of PD-1 and/or PD-L1 as compared to levels prior to treatment (e.g. baseline level).

In one embodiment, the methods of the present invention further comprise administering at least one neo-plastic agent or cancer adjuvant to said human. The methods of the present invention may also be employed with other therapeutic methods of cancer treatment. Typically, any anti-neoplastic agent or cancer adjuvant that has activity versus a tumor, such as a susceptible tumor being treated may be co-administered in the treatment of cancer in the present invention. Examples of such agents can be found in Cancer Principles and Practice of Oncology by V. T. Devita, T. S. Lawrence, and S. A. Rosenberg (editors), 10th edition (Dec. 5, 2014), Lippincott Williams & Wilkins Publishers.

In one embodiment, the human has previously been treated with one or more different cancer treatment modalities. In some embodiments, at least some of the patients in the cancer patient population have previously been treated with one or more therapies, such as surgery, radiotherapy, chemotherapy or immunotherapy. In some embodiments, at least some of the patients in the cancer patient population have previously been treated with chemotherapy (e.g. platinum-based chemotherapy). For example, a patient who has received two lines of cancer treatment can be identified as a 2 L cancer patient (e.g. a 2 L NSCLC patient). In some embodiments, a patient has received two lines or more lines of cancer treatment (e.g. a 2 L+ cancer patient such as a 2 L+ endometrial cancer patient). In some embodiments, a patient has not been previously treated with an antibody therapy, such as an anti-PD-1 therapy. In some embodiments, a patient previously received at least one line of cancer treatment (e.g. a patient previously received at least one line or at least two lines of cancer treatment). In some embodiments, a patient previously received at least one line of treatment for metastatic cancer (e.g. a patient previously received one or two lines of treatment for metastatic cancer). In some embodiments, a subject is resistant to treatment with a PD-1 inhibitor. In some embodiments, a subject is refractory to treatment with a PD-1 inhibitor. In some embodiments, a method described herein sensitizes the subject to treatment with a PD-1 inhibitor.

In certain embodiments, the cancer to be treated is PD-L1 positive. For example, in certain embodiments, the cancer to be treated exhibits PD-L1+ expression (e.g., high PD-L1 expression). Methods of detecting a biomarker, such as PD-L1 for example, on a cancer or tumor, are routine in the art and are contemplated herein. Non-limiting examples include immunohistochemistry, immunofluorescence and fluorescence activated cell sorting (FACS).

In various embodiments, the method of the invention is employed as a first, second, third or later line of treatment. A line of treatment refers to a place in the order of treatment with different medications or other therapies received by a patient. First-line therapy regimens are treatments given first, whereas second- or third-line therapy is given after the first-line therapy or after the second-line therapy, respectively. Therefore, first-line therapy is the first treatment for a disease or condition. In patients with cancer, first-line therapy, sometimes referred to as primary therapy or primary treatment, can be surgery, chemotherapy, radiation therapy, or a combination of these therapies. Typically, a patient is given a subsequent chemotherapy regimen (second- or third-line therapy), either because the patient did not show a positive clinical outcome or only showed a sub-clinical response to a first- or second-line therapy or showed a positive clinical response but later experienced a relapse, sometimes with disease now resistant to the earlier therapy that elicited the earlier positive response.

If the safety and the clinical benefit offered by the therapeutic combination of the invention are confirmed, this combination of a PD-1 inhibitor, a TGFβ inhibitor, an ATM inhibitor and radiotherapy warrants a first-line setting in cancer patients.

In some embodiments, the therapeutic combination of the invention is applied in a later line of treatment, particularly a second-line or higher treatment of the cancer. There is no limitation to the prior number of therapies provided that the subject underwent at least one round of prior cancer therapy. The round of prior cancer therapy refers to a defined schedule/phase for treating a subject with, e.g., one or more chemotherapeutic agents, radiotherapy or chemoradiotherapy, and the subject failed with such previous treatment, which was either completed or terminated ahead of schedule. One reason could be that the cancer was resistant or became resistant to prior therapy. The current standard of care (SoC) for treating cancer patients often involves the administration of toxic and old chemotherapy regimens. The SoC is associated with high risks of strong adverse events that are likely to interfere with the quality of life (such as secondary cancers). The toxicity profile of the therapeutic combination of the invention is expected to be much better than the SoC chemotherapy. In one embodiment, the combined administration of the PD-1 inhibitor, TGFβ inhibitor, ATM inhibitor and radiotherapy, may be as effective and better tolerated than SoC chemotherapy in patients with cancer resistant to mono- and/or poly-chemotherapy, radiotherapy or chemoradiotherapy. As the modes of action of the PD-1 inhibitor, TGFβ inhibitor, ATM inhibitor and radiotherapy are different, it is thought that the likelihood that administration of the therapeutic treatment of the invention may lead to enhanced immune-related adverse events (irAE) is small.

In a preferred embodiment, the PD-1 inhibitor, TGFβ inhibitor, ATM inhibitor and radiotherapy are administered in a second-line or higher treatment, more preferably a second-line treatment, of the cancer selected from the group of pre-treated relapsing metastatic NSCLC, unresectable locally advanced NSCLC, pre-treated SCLC ED, SCLC unsuitable for systemic treatment, pre-treated relapsing (recurrent) or metastatic SCCHN, recurrent SCCHN eligible for re-irradiation, and pre-treated microsatellite status instable low (MSI-L) or microsatellite status stable (MSS) metastatic colorectal cancer (mCRC). SCLC and SCCHN are particularly systemically pre-treated. MSI-L/MSS mCRC occurs in 85% of all mCRC.

In certain embodiments that employ an anti-PD-L1/TGFβ Trap in the combination therapy, the dosing regimen comprises administering the anti-PD-L1/TGFβ Trap at a dose of about 1200 mg to about 3000 mg (e.g., about 1200 mg to about 3000 mg, about 1200 mg to about 2900 mg, about 1200 mg to about 2800 mg, about 1200 mg to about 2700 mg, about 1200 mg to about 2600 mg, about 1200 mg to about 2500 mg, about 1200 mg to about 2400 mg, about 1200 mg to about 2300 mg, about 1200 mg to about 2200 mg, about 1200 mg to about 2100 mg, about 1200 mg to about 2000 mg, about 1200 mg to about 1900 mg, about 1200 mg to about 1800 mg, about 1200 mg to about 1700 mg, about 1200 mg to about 1600 mg, about 1200 mg to about 1500 mg, about 1200 mg to about 1400 mg, about 1200 mg to about 1300 mg, about 1300 mg to about 3000 mg, about 1400 mg to about 3000 mg, about 1500 mg to about 3000 mg, about 1600 mg to about 3000 mg, about 1700 mg to about 3000 mg, about 1800 mg to about 3000 mg, about 1900 mg to about 3000 mg, about 2000 mg to about 3000 mg, about 2100 mg to about 3000 mg, about 2200 mg to about 3000 mg, about 2300 mg to about 3000 mg, about 2400 mg to about 3000 mg, about 2500 mg to about 3000 mg, about 2600 mg to about 3000 mg, about 2700 mg to about 3000 mg, about 2800 mg to about 3000 mg, about 2900 mg to about 3000 mg, about 1200, about 1300, about 1400, about 1500, about 1600, about 1700, about 1800, about 1900, about 2000, about 2100, about 2200, about 2300, about 2400, about 2500 mg, about 2600 mg, about 2700 mg, about 2800 mg, about 2900 mg, or about 3000 mg). In certain embodiments, about 1200 mg of anti-PD-L1/TGFβ Trap molecule is administered to a subject once every two weeks. In certain embodiments, about 1800 mg of anti-PD-L1/TGFβ Trap molecule is administered to a subject once every three weeks. In certain embodiments, about 2400 mg of anti-PD-L1/TGFβ Trap molecule is administered to a subject once every three weeks.

In certain embodiments that employ an ATM inhibitor, such as Compound A, A1 or A2, in the combination therapy, the dosing regimen comprises administering the ATM inhibitor at a dose of about 1 to 1000 mg, e.g., about 1 mg to about 900 mg, about 1 mg to about 800 mg, about 1 mg to about 800 mg, about 1 mg to about 700 mg, about 1 mg to about 600 mg, about 1 mg to about 500 mg, about 1 mg to about 400 mg, about 1 mg to about 300 mg, about 1 mg to about 200 mg, about 1 mg to about 100 mg, about 10 mg to about 1000 mg, about 10 mg to about 900 mg, about 10 mg to about 800 mg, about 10 mg to about 700 mg, about 10 mg to about 600 mg, about 10 mg to about 500 mg, about 10 mg to about 400 mg, about 10 mg to about 300 mg, about 20 mg to about 1000 mg, about 200 mg to about 900 mg, about 20 mg to about 800 mg, about 20 mg to about 700 mg, about 20 mg to about 600 mg, about 20 mg to about 500 mg, about 20 mg to about 400 mg, about 20 mg to about 300 mg, about 20 mg to about 200 mg, about 25 mg to about 1000 mg, about 25 mg to about 900 mg, about 25 mg to about 800 mg, about 25 mg to about 700 mg, about 25 mg to about 600 mg, about 25 mg to about 500 mg, about 25 mg to about 400 mg, about 25 mg to about 300 mg). In certain embodiments, about 25 to 500 mg ATM inhibitor, most preferably Compound A or A1, is administered to a subject daily, preferably once daily. In certain embodiments, about 25 to 350 mg Compound A1 is administered to a subject daily. In certain embodiments, about 25, 50, 75, 100, 125, 150, 175, 200, 225, 250, 275, 300, 325, 350, 375, or 400 mg of ATM inhibitor, preferably Compound A or A1, is administered to a subject daily.

In certain embodiments, the radiotherapy comprises about 35-70 Gy/20-35 fractions. In some embodiments, the radiotherapy is given either with standard fractionation (1.8 to 2 Gy per day for 5 days a week) up to a total dose of 50-70 Gy. Other fractionation schedules could also be envisioned, for example, a lower dose per fraction but given twice daily. Higher daily doses over a shorter period of time can also be given. In one embodiment, stereotactic radiotherapy as well as the gamma knife are used. In the palliative setting, other fractionation schedules are also widely used for example 25 Gy in 5 fractions or 30 Gy in 10 fractions. For radiotherapy, the duration of treatment will be the time frame when radiotherapy is given. These interventions apply to treatment given with electrons, photons and protons, alfa-emitters or other ions, treatment with radio-nucleotides, for example, treatment with¹³¹ I given to patients with thyroid cancer, as well in patients treated with boron capture neutron therapy.

Concurrent treatment considered necessary for the patient's well-being may be given at discretion of the treating physician. In some embodiments, the present invention provides methods of treating, stabilizing or decreasing the severity or progression of one or more diseases or disorders described herein comprising administering to a patient in need thereof a PD-1 inhibitor, a TGFβ inhibitor, an ATM inhibitor and radiotherapy in combination with an additional therapeutic agent, such as a chemotherapeutic agent. In certain embodiments, the chemotherapeutic agent is selected from the group of etoposide, doxorubicin, topotecan, irinotecan, fluorouracil, a platin, an anthracycline, and a combination thereof.

The PD-1 inhibitor, TGFβ inhibitor, ATM inhibitor and radiotherapy are administered using any amount and any route of administration effective for treating or decreasing the severity of a disorder provided above. The exact amount required will vary from subject to subject, depending on the species, age, and general condition of the subject, the severity of the infection, the particular agent, its mode of administration, and the like.

In some embodiments, the PD-1 inhibitor, TGFβ inhibitor, ATM inhibitor and radiotherapy are administered simultaneously, separately or sequentially and in any order. The PD-1 inhibitor, TGFβ inhibitor, ATM inhibitor and radiotherapy are administered to the patient in any order (i.e., simultaneously or sequentially) and the compounds may be in separate compositions, formulations or unit dosage forms, or together in a single composition, formulation or unit dosage form. In one embodiment, the PD-1 inhibitor, TGFβ inhibitor, ATM inhibitor and radiotherapy are administered simultaneously or sequentially in any order, in jointly therapeutically effective amounts, (for example in synergistically effective amounts), e.g. in daily or intermittently dosages corresponding to the amounts described herein. The individual combination partners of the PD-1 inhibitor, TGFβ inhibitor, ATM inhibitor and radiotherapy may be administered separately at different times during the course of therapy or concurrently. Typically, in such combination therapies, individual compounds are formulated into separate pharmaceutical compositions or medicaments. When the compounds are separately formulated, the individual compounds and the radiation can be administered simultaneously or sequentially, optionally via different routes in case of the compounds. Optionally, the treatment regimens for each of the PD-1 inhibitor, TGFβ inhibitor, ATM inhibitor and radiotherapy have different but overlapping delivery regimens, e.g., daily, twice daily, vs. a single administration, or weekly. In certain embodiments, the PD-1 inhibitor, TGFβ inhibitor and ATM inhibitor are administered simultaneously in the same composition comprising the PD-1 inhibitor, TGFβ inhibitor and ATM inhibitor. In certain embodiments, the PD-1 inhibitor, TGFβ inhibitor and ATM inhibitor are administered simultaneously in separate compositions, i.e., wherein the PD-1 inhibitor, TGFβ inhibitor and ATM inhibitor are administered simultaneously each in a separate unit dosage form. Preferably, the PD-1 inhibitor and TGFβ inhibitor are fused and administered in a separate unit dosage form from the ATM inhibitor and the PD-1 inhibitor and TGFβ inhibitor are administered simultaneously or sequentially in any order with the ATM inhibitor and radiotherapy. It will be appreciated that the PD-1 inhibitor, TGFβ inhibitor, ATM inhibitor and radiotherapy are administered on the same day or on different days and in any order as according to an appropriate dosing protocol. The instant invention is therefore to be understood as embracing all such regimens of simultaneous or alternating treatment and the term “administering” is to be interpreted accordingly.

In some embodiments, the anti-PD-L1/TGFβ Trap, ATM inhibitor and radiotherapy are administered simultaneously, separately or sequentially and in any order. The anti-PD-L1/TGFβ Trap, ATM inhibitor and radiotherapy are administered to the patient in any order (i.e., simultaneously or sequentially) in separate compositions, formulations or unit dosage forms, or together in a single composition, formulation or unit dosage form. In one embodiments, a method of treating a proliferative disease may comprise administration of a combination of an anti-PD-L1/TGFβ Trap, an ATM inhibitor and radiotherapy wherein the individual combination partners are administered simultaneously or sequentially in any order, in jointly therapeutically effective amounts, (for example in synergistically effective amounts), e.g. in daily or intermittently dosages corresponding to the amounts described herein. The individual combination partners of the anti-PD-L1/TGFβ Trap, ATM inhibitor and radiotherapy may be administered separately at different times during the course of therapy or concurrently in divided or single combination forms. Typically, in such combination therapies, the individual compounds are formulated into separate pharmaceutical compositions or medicaments. When separately formulated, the individual compounds can be administered simultaneously or sequentially, optionally via different routes. Optionally, the treatment regimens for each of the anti-PD-L1/TGFβ Trap, ATM inhibitor and radiotherapy have different but overlapping delivery regimens, e.g., daily, twice daily, vs. a single administration, or weekly. The anti-PD-L1/TGFβ Trap may be delivered prior to, substantially simultaneously with, or after, the ATM inhibitor and/or radiotherapy. In certain embodiments, the anti-PD-L1/TGFβ Trap is administered simultaneously in the same composition comprising the anti-PD-L1/TGFβ Trap and ATM inhibitor. In certain embodiments, the anti-PD-L1/TGFβ Trap and ATM inhibitor are administered simultaneously in separate compositions, i.e., wherein the anti-PD-L1/TGFβ Trap and ATM inhibitor are administered simultaneously each in a separate unit dosage form. It will be appreciated that the anti-PD-L1/TGFβ Trap, ATM inhibitor and radiotherapy are administered on the same day or on different days and in any order as according to an appropriate dosing protocol. The instant invention is therefore to be understood as embracing all such regimens of simultaneous or alternating treatment and the term “administering” is to be interpreted accordingly.

In some embodiments, the combination regimen comprises the steps of: (a) under the direction or control of a physician, the subject receiving the fused PD-1 inhibitor and TGFβ inhibitor, as well as the ATM inhibitor, prior to first receipt of radiotherapy; and (b) under the direction or control of a physician, the subject receiving radiotherapy. In some embodiments, the combination regimen comprises the steps of: (a) under the direction or control of a physician, the subject receiving the radiotherapy prior to first receipt of the fused PD-1 inhibitor and TGFβ inhibitor, as well as the ATM inhibitor; and (b) under the direction or control of a physician, the subject receiving the fused PD-1 inhibitor and TGFβ inhibitor, as well as the ATM inhibitor. In some embodiments, the combination regimen comprises the steps of: (a) under the direction or control of a physician, the subject receiving the radiotherapy, as well as the ATM inhibitor, prior to first receipt of the fused PD-1 inhibitor and TGFβ inhibitor; and (b) under the direction or control of a physician, the subject receiving the fused PD-1 inhibitor and TGFβ inhibitor. In some embodiments, the combination regimen comprises the steps of: (a) under the direction or control of a physician, the subject receiving the fused PD-1 inhibitor and TGFβ inhibitor prior to first receipt of radiotherapy and the ATM inhibitor; and (b) under the direction or control of a physician, the subject receiving radiotherapy and the ATM inhibitor. In some embodiments, the combination regimen comprises the steps of: (a) under the direction or control of a physician, the subject receiving the fused PD-1 inhibitor and TGFβ inhibitor prior to first receipt of the radiotherapy, as well as the ATM inhibitor; and (b) under the direction or control of a physician, the subject receiving radiotherapy, as well as the ATM inhibitor. In some embodiments, the combination regimen comprises the steps of: (a) under the direction or control of a physician, the subject receiving the fused PD-1 inhibitor and TGFβ inhibitor, as well as radiotherapy, prior to first receipt of the ATM inhibitor; and (b) under the direction or control of a physician, the subject receiving the ATM inhibitor. In some embodiments, the combination regimen comprises the steps of: (a) under the direction or control of a physician, the subject receiving the ATM inhibitor, as well as radiotherapy, prior to first receipt of the fused PD-1 inhibitor and TGFβ inhibitor; and (b) under the direction or control of a physician, the subject receiving the fused PD-1 inhibitor and TGFβ inhibitor.

Also provided is a combination comprising a PD-1 inhibitor, a TGFβ inhibitor and an ATM inhibitor. Also provided is a combination comprising an anti-PD-L1/TGFβ Trap and an ATM inhibitor. In some embodiments, the combination comprising a PD-1 inhibitor, a TGFβ inhibitor and an ATM inhibitor or the combination comprising an anti-PD-L1/TGFβ Trap and an ATM inhibitor is for use as a medicament in further combination with radiotherapy or for use in the treatment of cancer in further combination with radiotherapy.

It shall be understood that, in the various embodiments described above, the PD-1 inhibitor and the TGFβ inhibitor are preferably fused and, more preferably, correspond to an anti-PD-L1/TGFβ Trap.

### Pharmaceutical Formulations and Kits

In some embodiments, the present invention provides a pharmaceutically acceptable composition comprising a PD-1 inhibitor. In some embodiments, the present invention provides a pharmaceutically acceptable composition comprising a TGFβ inhibitor. In some embodiments, the present invention provides a pharmaceutically acceptable composition comprising anti-PD-L1/TGFβ Trap. In some embodiments, the present invention provides a pharmaceutically acceptable composition comprising an ATM inhibitor, preferably Compound A, or a pharmaceutically acceptable salt thereof. In some embodiments, the present invention provides a pharmaceutically acceptable composition of a chemotherapeutic agent. In some embodiments, the present invention provides a pharmaceutical composition comprising a PD-1 inhibitor and a TGFβ inhibitor. In some embodiments, the present invention provides a pharmaceutical composition comprising a TGFβ inhibitor and an ATM inhibitor. In some embodiments, the present invention provides a pharmaceutical composition comprising a PD-1 inhibitor and an ATM inhibitor. In some embodiments, the present invention provides a pharmaceutical composition comprising an anti-PD-L1/TGFβ Trap and an ATM inhibitor. In some embodiments, the present invention provides a pharmaceutical composition comprising a PD-1 inhibitor, a TGFβ inhibitor and an ATM inhibitor. The pharmaceutically acceptable composition may comprise at least a further pharmaceutically acceptable excipient or adjuvant, such as a pharmaceutically acceptable carrier.

In some embodiments, a composition comprising the fused PD-1 inhibitor and TGFβ inhibitor, e.g., an anti-PD-L1/TGFβ Trap, is separate from a composition comprising an ATM inhibitor, e.g., Compound A. In some embodiments, the PD-1 inhibitor and TGFβ inhibitor are fused e.g., as an anti-PD-L1/TGFβ Trap, and present with an ATM inhibitor, preferably Compound A in the same composition.

Examples of such pharmaceutically acceptable compositions are described further below and herein.

The compositions of the present invention may be in a variety of forms. These include, for example, liquid, semi-solid and solid dosage forms, such as liquid solutions (e.g., injectable and infusible solutions), dispersions or suspensions, tablets, pills, powders, liposomes, and suppositories. Compositions of the present invention are administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally, subcutaneously or intravenously. In a preferred embodiment, the PD-1 inhibitor or TGFβ inhibitor is administered by intravenous infusion or injection. In another preferred embodiment, the PD-1 inhibitor or TGFβ inhibitor is administered by intramuscular or subcutaneous injection. In a preferred embodiment, anti-PD-L1/TGFβ Trap is administered by intravenous infusion or injection. In another preferred embodiment, anti-PD-L1/TGFβ Trap is administered by intramuscular or subcutaneous injection. In a preferred embodiment, the ATM inhibitor is administered orally.

In some embodiments, anti-PD-L1/TGFβ Trap is administered intravenously (e.g., as an intravenous infusion) or subcutaneously, preferably intravenously. More preferably, anti-PD-L1/TGFβ Trap is administered as an intravenous infusion. In some embodiments, anti-PD-L1/TGFβ Trap is administered at a dose of about 1200 mg, 1800 mg or 2400 mg. In some embodiments, anti-PD-L1/TGFβ Trap is administered at a dose of about 1200 mg, 1800 mg or 2400 mg once every two weeks (Q2WV) or once every three weeks (Q3WV).

Pharmaceutically acceptable carriers, adjuvants or vehicles that are used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Liquid dosage forms for oral administration include, but are not limited to, pharmaceutically acceptable emulsions, microemulsions, solutions, suspensions, syrups and elixirs. The liquid dosage forms may additionally contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S. P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the compounds of the invention, it is often desirable to slow absorption from subcutaneous or intramuscular injection. This may be accomplished by the use of a liquid suspension of crystalline or amorphous material with poor water solubility. The rate of absorption then depends upon its rate of dissolution that, in turn, may depend upon crystal size and crystalline form. Alternatively, delayed absorption of parenterally administered PD-1 inhibitor, TGFβ inhibitor and/or ATM inhibitor, is accomplished by dissolving or suspending the compound in an oil vehicle. Injectable depot forms are made by forming microencapsulated matrices of PD-1 inhibitor, TGFβ inhibitor and/or ATM inhibitor in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories, which can be prepared by mixing the compounds of this invention with suitable non-irritating excipients or carriers such as cocoa butter, polyethylene glycol or a suppository wax, which are solid at ambient temperature but liquid at body temperature and therefore melt in the rectum or vaginal cavity and release the active compound.

Dosage forms for oral administration include capsules, tablets, pills, powders, and granules, aqueous suspensions or solutions. In solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol and silicic acid, b) binders such as, for example, carboxymethylcellulose, alginates, gelatin, polyvinylpyrrolidinone, sucrose and acacia, c) humectants such as glycerol, d) disintegrating agents such as agar-agar, calcium carbonate, potato or tapioca starch, alginic acid, certain silicates and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hardfilled gelatin capsules using such excipients as lactose or milk sugar as well as high molecular weight polyethylene glycols and the like. The solid dosage forms of tablets, dragées, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The PD-1 inhibitor, TGFβ inhibitor and/or ATM inhibitor can also be in micro-encapsulated form with one or more excipients as noted above. The solid dosage forms of tablets, dragées, capsules, pills, and granules can be prepared with coatings and shells such as enteric coatings, release controlling coatings and other coatings well known in the pharmaceutical formulating art. In such solid dosage forms, the PD-1 inhibitor, TGFβ inhibitor and/or ATM inhibitor may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of the PD-1 inhibitor, TGFβ inhibitor and/or ATM inhibitor include ointments, pastes, creams, lotions, gels, powders, solutions, sprays, inhalants or patches. The active component is admixed under sterile conditions with a pharmaceutically acceptable carrier and any needed preservatives or buffers as may be required. Exemplary carriers for topical administration of compounds of this are mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Pharmaceutically acceptable compositions of this invention are optionally administered by nasal aerosol or inhalation. Such compositions are prepared according to techniques well-known in the art of pharmaceutical formulation and are prepared as solutions in saline, employing benzyl alcohol or other suitable preservatives, absorption promoters to enhance bioavailability, fluorocarbons, and/or other conventional solubilizing or dispersing agents.

In a further aspect, the invention relates to a kit comprising a PD-1 inhibitor and a package insert comprising instructions for using the PD-1 inhibitor in combination with an ATM inhibitor, a TGFβ inhibitor and radiotherapy to treat or delay progression of a cancer in a subject. Also provided is a kit comprising an ATM inhibitor and a package insert comprising instructions for using the ATM inhibitor in combination with a PD-1 inhibitor, a TGFβ inhibitor and radiotherapy to treat or delay progression of a cancer in a subject. Also provided is a kit comprising a TGFβ inhibitor and a package insert comprising instructions for using the TGFβ inhibitor in combination with a PD-1 inhibitor, an ATM inhibitor and radiotherapy to treat or delay progression of a cancer in a subject. Also provided is a kit comprising anti-PD-L1/TGFβ Trap and a package insert comprising instructions for using the anti-PD-L1/TGFβ Trap in combination with an ATM inhibitor and radiotherapy to treat or delay progression of a cancer in a subject. Also provided is a kit comprising a PD-1 inhibitor and an ATM inhibitor, and a package insert comprising instructions for using the PD-1 inhibitor and the ATM inhibitor in combination with a TGFβ inhibitor and radiotherapy to treat or delay progression of a cancer in a subject. Also provided is a kit comprising a TGFβ inhibitor and an ATM inhibitor, and a package insert comprising instructions for using the TGFβ inhibitor and the ATM inhibitor in combination with a PD-1 inhibitor and radiotherapy to treat or delay progression of a cancer in a subject. Also provided is a kit comprising a PD-1 inhibitor and a TGFβ inhibitor, and a package insert comprising instructions for using the PD-1 inhibitor and the TGFβ inhibitor in combination with an ATM inhibitor and radiotherapy to treat or delay progression of a cancer in a subject.

Also provided is a kit comprising anti-PD-L1/TGFβ Trap and an ATM inhibitor, and a package insert comprising instructions for using anti-PD-L1/TGFβ Trap, the ATM inhibitor and radiotherapy to treat or delay progression of a cancer in a subject. Also provided is a kit comprising a PD-1 inhibitor, a TGFβ inhibitor and an ATM inhibitor, and a package insert comprising instructions for using the PD-1 inhibitor, the TGFβ inhibitor, the ATM inhibitor and radiotherapy to treat or delay progression of a cancer in a subject. The kit can comprise a first container, a second container, a third container and a package insert, wherein the first container comprises at least one dose of the PD-1 inhibitor, the second container comprises at least one dose of the ATM inhibitor, the third container comprises at least one dose of the TGFβ inhibitor and the package insert comprises instructions for treating a subject for cancer using the three compounds and radiotherapy. In some embodiments, the kit comprises a first container, a second container and a package insert, wherein the first container comprises at least one dose of anti-PD-L1/TGFβ Trap, the second container comprises at least one dose of the ATM inhibitor and the package insert comprises instructions for treating a subject for cancer using the two compounds and radiotherapy. The first, second and third containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. The instructions can state that the medicaments are intended for use in treating a subject having a cancer that tests positive for PD-L1, e.g., by means of an immunohistochemical (IHC) assay, FACS or LC/MS/MS.

Further Diagnostic, Predictive, Prognostic and/or Therapeutic Methods

The disclosure further provides diagnostic, predictive, prognostic and/or therapeutic methods, which are based, at least in part, on determination of the identity of the expression level of a marker of interest. In particular, the amount of human PD-L1 in a cancer patient sample can be used to predict whether the patient is likely to respond favorably to cancer therapy utilizing the therapeutic combination of the invention. In some embodiments, the amount of human TGFβ in a cancer patient sample, preferably a serum sample, can be used to predict whether the patient is likely to respond favorably to cancer therapy utilizing the therapeutic combination of the invention.

Any suitable sample can be used for the method. Non-limiting examples of such include one or more of a serum sample, plasma sample, whole blood, pancreatic juice sample, tissue sample, tumor lysate or a tumor sample, which can be an isolated from a needle biopsy, core biopsy and needle aspirate. For example, tissue, plasma or serum samples are taken from the patient before treatment and optionally on treatment with the therapeutic combination of the invention. The expression levels obtained on treatment are compared with the values obtained before starting treatment of the patient. The information obtained may be prognostic in that it can indicate whether a patient has responded favorably or unfavorably to cancer therapy.

It is to be understood that information obtained using the diagnostic assays described herein may be used alone or in combination with other information, such as, but not limited to, expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient, and the like. In a particular aspect, the expression level can be used in a diagnostic panel each of which contributes to the final diagnosis, prognosis, or treatment selected for a patient.

Any suitable method can be used to measure the PD-L1 or TGFβ protein, DNA, RNA, or other suitable read-outs for PD-L1 or TGFβ levels, examples of which are described herein and/or are well known to the skilled artisan.

In some embodiments, determining the PD-L1 or TGFβ level comprises determining the PD-L1 or TGFβ expression. In some preferred embodiments, the PD-L1 or TGFβ level is determined by the PD-L1 or TGFβ protein concentration in a patient sample, e.g., with PD-L1 or TGFβ specific ligands, such as antibodies or specific binding partners. The binding event can, e.g., be detected by competitive or non-competitive methods, including the use of a labeled ligand or PD-L1 or TGFβ specific moieties, e.g., antibodies, or labeled competitive moieties, including a labeled PD-L1 or TGFβ standard, which compete with marker proteins for the binding event. If the marker specific ligand is capable of forming a complex with PD-L1 or TGFβ, the complex formation can indicate PD-L1 or TGFβ expression in the sample. In various embodiments, the biomarker protein level is determined by a method comprising quantitative western blot, multiple immunoassay formats, ELISA, immunohistochemistry, histochemistry, or use of FACS analysis of tumor lysates, immunofluorescence staining, a bead-based suspension immunoassay, Luminex technology, or a proximity ligation assay. In a preferred embodiment, the PD-L1 or TGFβ expression is determined by immunohistochemistry using one or more primary anti-PD-L1 or anti-TGFβ antibodies. In another embodiment, the biomarker RNA level is determined by a method comprising microarray chips, RT-PCR, qRT-PCR, multiplex qPCR or in-situ hybridization. In one embodiment of the invention, a DNA or RNA array comprises an arrangement of poly-nucleotides presented by or hybridizing to the PD-L1 or TGFβ gene immobilized on a solid surface. For example, to the extent of determining the PD-L1 or TGFβ mRNA, the mRNA of the sample can be isolated, if necessary, after adequate sample preparation steps, e.g., tissue homogenization, and hybridized with marker specific probes, in particular on a microarray platform with or without amplification, or primers for PCR-based detection methods, e.g., PCR extension labeling with probes specific for a portion of the marker mRNA.

Several approaches have been described for quantifying PD-L1 protein expression in IHC assays of tumor tissue sections (Thompson et al. (2004) PNAS 101(49): 17174; Thompson et al. (2006) Cancer Res. 66: 3381; Gadiot et al. (2012) Cancer 117: 2192; Taube et al. (2012) Sci Transl Med 4, 127ra37; and Toplian et al. (2012) New Eng. J Med. 366 (26): 2443). One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression is at least 1%, and preferably 5% of total tumor cells.

The level of PD-L1 or TGFβ mRNA expression may be compared to the mRNA expression levels of one or more reference genes that are frequently used in quantitative RT-PCR, such as ubiquitin C. In some embodiments, a level of PD-L1 or TGFβ expression (protein and/or mRNA) by malignant cells and/or by infiltrating immune cells within a tumor is determined to be “overexpressed” or “elevated” based on comparison with the level of PD-L1 or TGFβ expression (protein and/or mRNA) by an appropriate control. For example, a control PD-L1 or TGFβ protein or mRNA expression level may be the level quantified in non-malignant cells of the same type or in a section from a matched normal tissue.

In a preferred embodiment, the efficacy of the therapeutic combination of the invention is predicted by means of PD-L1 or TGFβ expression in tumor samples. Immunohistochemistry with anti-PD-L1 or anti-TGFβ primary antibodies can be performed on serial cuts of formalin fixed and paraffin embedded specimens from patients treated with an anti-PD-L1 antibody or an anti-TGFβ antibody.

This disclosure also provides a kit for determining if the combination of the invention is suitable for therapeutic treatment of a cancer patient, comprising means for determining a protein level of PD-L1 or TGFβ, or the expression level of its RNA, in a sample isolated from the patient and instructions for use. In another aspect, the kit further comprises an anti-PD-L1 antibody for immunotherapy. In one aspect of the invention, the determination of a high PD-L1 or TGFβ level indicates increased PFS or OS when the patient is treated with the therapeutic combination of the invention. In one embodiment of the kit, the means for determining the PD-L1 or TGFβ protein level are antibodies with specific binding to PD-L1 or TGFβ, respectively.

In still another aspect, the invention provides a method for advertising a PD-1 inhibitor in combination with a TGFβ inhibitor, an ATM inhibitor and radiotherapy, comprising promoting, to a target audience, the use of the combination for treating a subject with a cancer, optionally, based on PD-L1 and/or TGFβ expression in samples taken from the subject. In still another aspect, the invention provides a method for advertising an ATM inhibitor in combination with radiotherapy, a PD-1 inhibitor and a TGFβ inhibitor, wherein the PD-1 inhibitor and TGFβ inhibitor are preferably fused, comprising promoting, to a target audience, the use of the combination for treating a subject with a cancer, optionally, based on PD-L1 and/or TGFβ expression in samples taken from the subject. In still another aspect, the invention provides a method for advertising a TGFβ inhibitor in combination with a PD-1 inhibitor, an ATM inhibitor and radiotherapy, comprising promoting, to a target audience, the use of the combination for treating a subject with a cancer, optionally, based on PD-L1 and/or TGFβ expression in samples taken from the subject. In still another aspect, the invention provides a method for advertising an anti-PD-L1/TGFβ Trap in combination with an ATM inhibitor and radiotherapy, comprising promoting, to a target audience, the use of the combination for treating a subject with a cancer, optionally, based on PD-L1 and/or TGFβ expression in samples taken from the subject. In still another aspect, the invention provides a method for advertising a combination comprising a PD-1 inhibitor, a TGFβ inhibitor and an ATM inhibitor in combination with radiotherapy, comprising promoting, to a target audience, the use of the combination (including the radiotherapy) for treating a subject with a cancer, optionally, based on PD-L1 and/or TGFβ expression in samples taken from the subject. Promotion may be conducted by any means available. In some embodiments, the promotion is by a package insert accompanying a commercial formulation of the therapeutic combination of the invention. The promotion may also be by a package insert accompanying a commercial formulation of the PD-1 inhibitor, TGFβ inhibitor, ATM inhibitor or another medicament (when treatment is a therapy with the therapeutic combination of the invention and a further medicament). In some embodiments, the promotion is by a package insert where the package insert provides instructions to receive therapy with the therapeutic combination of the invention after measuring PD-L1 and/or TGFβ expression levels, and in some embodiments, in combination with another medicament. In some embodiments, the promotion is followed by the treatment of the patient with the therapeutic combination of the invention with or without another medicament. In some embodiments, the package insert indicates that the therapeutic combination of the invention is to be used to treat the patient if the patient's cancer sample is characterized by high PD-L1 and/or high TGFβ biomarker levels. In some embodiments, the package insert indicates that the therapeutic combination of the invention is not to be used to treat the patient if the patient's cancer sample expresses low PD-L1 and/or low TGFβ biomarker levels. In some embodiments, a high PD-L1 and/or high TGFβ biomarker level means a measured PD-L1 and/or TGFβ level that correlates with a likelihood of increased PFS and/or OS when the patient is treated with the therapeutic combination of the invention, and vice versa. In some embodiments, the PFS and/or OS is decreased relative to a patient who is not treated with the therapeutic combination of the invention. In some embodiments, the promotion is by a package insert where the package inset provides instructions to receive therapy with anti-PD-L1/TGFβ Trap in combination with an ATM inhibitor and radiotherapy after first measuring PD-L1 and/or TGFβ. In some embodiments, the promotion is followed by the treatment of the patient with anti-PD-L1/TGFβ Trap in combination with an ATM inhibitor and radiotherapy with or without another medicament. Further methods of advertising and instructing, or business methods applicable in accordance with the invention are described (for other drugs and biomarkers) in US 2012/0089541, for example.

The following embodiments are preferred:
1. A PD-1 inhibitor, a TGFβ inhibitor and an ATM inhibitor for use in a method of treating a cancer in a subject, wherein the method comprises administering the PD-1 inhibitor, the TGFβ inhibitor and the ATM inhibitor to the subject in combination with radiotherapy.
2. A PD-1 inhibitor for use in a method of treating a cancer in a subject, wherein the method comprises administering the PD-1 inhibitor to the subject in combination with a TGFβ inhibitor, an ATM inhibitor, and radiotherapy.
3. A TGFβ inhibitor for use in a method of treating a cancer in a subject, wherein the method comprises administering the TGFβ inhibitor to the subject in combination with a PD-1 inhibitor, an ATM inhibitor, and radiotherapy.
4. An ATM inhibitor for use in a method of treating a cancer in a subject, wherein the method comprises administering the ATM inhibitor to the subject in combination with a PD-1 inhibitor, a TGFβ inhibitor, and radiotherapy.
5. A PD-1 inhibitor and a TGFβ inhibitor for use in a method of treating a cancer in a subject, wherein the method comprises administering the PD-1 inhibitor and the TGFβ inhibitor to the subject in combination with an ATM inhibitor and radiotherapy; and
   wherein the PD-1 inhibitor and the TGFβ inhibitor are fused.
6. A method of treating a cancer in a subject, wherein the method comprises administering a PD-1 inhibitor, a TGFβ inhibitor and an ATM inhibitor to the subject in combination with radiotherapy.
7. Use of a PD-1 inhibitor, a TGFβ inhibitor and an ATM inhibitor for the manufacture of a medicament for a method of treating a cancer in a subject, wherein the method comprises administering the PD-1 inhibitor, the TGFβ inhibitor and the ATM inhibitor to the subject in combination with radiotherapy.
8. Use of a PD-1 inhibitor for the manufacture of a medicament for a method of treating a cancer in a subject, wherein the method comprises administering the PD-1 inhibitor to the subject in combination with a TGFβ inhibitor, an ATM inhibitor, and radiotherapy.
9. Use of a TGFβ inhibitor for the manufacture of a medicament for a method of treating a cancer in a subject, wherein the method comprises administering the TGFβ inhibitor to the subject in combination with a PD-1 inhibitor, an ATM inhibitor, and radiotherapy.
10. Use of an ATM inhibitor for the manufacture of a medicament for a method of treating a cancer in a subject, wherein the method comprises administering the ATM inhibitor to the subject in combination with a PD-1 inhibitor, a TGFβ inhibitor, and radiotherapy.
11. Use of a PD-1 inhibitor and a TGFβ inhibitor for the manufacture of a medicament for a method of treating a cancer in a subject, wherein the method comprises administering the PD-1 inhibitor and the TGFβ inhibitor to the subject in combination with an ATM inhibitor and radiotherapy; and
   wherein the PD-1 inhibitor and the TGFβ inhibitor are fused.
12. The compounds for use, method of treatment or use according to any one of items 1 to 11, wherein the PD-1 inhibitor is capable of inhibiting the interaction between PD-1 and PD-L1.
13. The compounds for use, method of treatment or use according to item 12, wherein the PD-1 inhibitor is an anti-PD-1 or anti-PD-L1 antibody.
14. The compounds for use, method of treatment or use according to item 13, wherein the PD-1 inhibitor is an anti-PD-L1 antibody.
15. The compounds for use, method of treatment or use according to item 14, wherein the anti-PD-L1 antibody comprises a heavy chain sequence, which comprises a CDR1 having the sequence of SEQ ID NO: 1, a CDR2 having the sequence of SEQ ID NO: 2 and a CDR3 having the sequence of SEQ ID NO: 3, and a light chain sequence, which comprises a CDR1 having the sequence of SEQ ID NO: 4, a CDR2 having the sequence of SEQ ID NO: 5 and a CDR3 having the sequence of SEQ ID NO: 6.
16. The compounds for use, method of treatment or use according to any one of items 1 to 15, wherein the TGFβ inhibitor is capable of inhibiting the interaction between a TGFβ and a TGFβ receptor.
17. The compounds for use, method of treatment or use according to any one of items 1 to 16, wherein the TGFβ inhibitor is a TGFβ receptor or a fragment thereof capable of binding TGFβ.
18. The compounds for use, method of treatment or use according to item 17, wherein the TGFβ receptor is TGFβ receptor II or a fragment thereof capable of binding TGFβ.
19. The compounds for use, method of treatment or use according to item 18, wherein the TGFβ receptor is an extracellular domain of TGFβ receptor II or a fragment thereof capable of binding TGFβ.
20. The compounds for use, method of treatment or use according to any one of items 1 to 19, wherein the TGFβ inhibitor has at least 80%, preferably 90%, more preferably 95%, sequence identity to the full-length amino acid sequence of any one of SEQ ID NO: 11, SEQ ID NO: 12 and SEQ ID NO: 13 and is capable of binding TGFβ.
21. The compounds for use, method of treatment or use according to any one of items 1 to 20, wherein the TGFβ inhibitor has at least 80% sequence identity to the full-length amino acid sequence of SEQ ID NO: 11 and is capable of binding TGFβ.
22. The compounds for use, method of treatment or use according to any one of items 1 to 19, wherein the TGFβ inhibitor comprises the sequence of any one of SEQ ID NO: 11, SEQ ID NO: 12 and SEQ ID NO: 13.
23. The compounds for use, method of treatment or use according to item 22, wherein the TGFβ inhibitor comprises the sequence of SEQ ID NO: 11.
24. The compounds for use, method of treatment or use according to any one of items 1 to 4, 6-10 and 12-23, wherein the PD-1 inhibitor and the TGFβ inhibitor are fused.
25. The compounds for use, method of treatment or use according to any one of items 5, 11 and 24, wherein the PD-1 inhibitor and the TGFβ inhibitor are fused in a molecule comprising (a) an antibody or a fragment thereof capable of binding PD-L1 and inhibiting the interaction between PD-1 and PD-L1 and (b) the extracellular domain of TGFβRII or a fragment thereof capable of binding TGFβ and inhibiting the interaction between TGFβ and a TGFβ receptor.
26. The compounds for use, method of treatment or use according to item 25, wherein the fusion molecule is one of the respective fusion molecules disclosed in WO 2015/118175 or WO 2018/205985.
27. The compounds for use, method of treatment or use according to item 25, the extracellular domain of the TGFβRII or the fragment thereof is fused to each of the heavy chain sequences of the antibody or the fragment thereof.
28. The compounds for use, method of treatment or use according to item 27, wherein the fusion between the extracellular domains of TGFβRII or fragments thereof and the heavy chain sequences of the antibody or the fragment thereof occurs via a linker sequence.
29. The compounds for use, method of treatment or use according to item 28, wherein the amino acid sequence of the light chain sequences and the sequences comprising the heavy chain sequence and the extracellular domain of TGFβRII or the fragment thereof respectively correspond to the sequences selected from the group consisting of: (1) SEQ ID NO: 7 and SEQ ID NO: 8, (2) SEQ ID NO: 15 and SEQ ID NO: 17, and (3) SEQ ID NO: 15 and SEQ ID NO: 18.
30. The compounds for use, method of treatment or use according to item 25, wherein the amino acid sequence of the fusion molecule is identical to the amino acid sequence of bintrafusp alfa.
31. The compounds for use, method of treatment or use according to any one of items 5, 11 and 24, wherein the fused PD-1 inhibitor and TGFβ inhibitor is bintrafusp alfa.
32. The compounds for use, method of treatment or use according to any one of items 1 to 31, wherein the ATM inhibitor has an IC₅₀ below 1 μM.
33. The compounds for use, method of treatment or use according to any one of item 1 to 32, wherein the ATM inhibitor is an imidazo[4,5-c]quinoline derivative.
34. The compounds for use, method of treatment or use according to item 33, wherein the ATM inhibitor is a compound of formula (I)

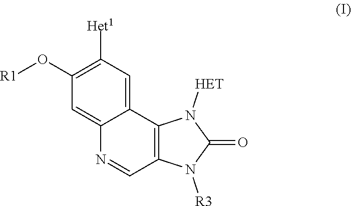

where
R1 denotes methyl,
R3 denotes methyl or H,
A in each case independently denotes unbranched or branched alkyl having 1, 2, 3, 4, 5, 6, 7, 8, 9 or 10 C atoms, where, independently of one another, 1, 2, 3, 4, 5, 6 or 7 H atoms may be replaced by Hal,
Het¹ is selected from the group consisting of pyridinyl, pyrimidinyl, pyrazolyl, triazolyl, imidazolyl, bezimidazolyl, imidazo[4,5-b]pyridinyl, and benzodiazolyl, each of which may be unsubstituted or mono-, di- or trisubstituted, independently of one another, by Hal, A, CN, —(CY₂)ₚ—OY, —(CY₂)ₚ—NYY, —(CY₂)ₚ—COOY, —(CY₂)ₚ—CO—NYY, —(CY₂)ₚ—NY—COY, —Het² and/or —SO₂—Het²,
Het² denotes a monocyclic saturated heterocycle having 2, 3, 4, 5, 6 or 7 C atoms and 1, 2, 3 or 4 N, O and/or S atoms, which may be unsubstituted or monosubstituted by A, HET denotes a 5- or 6-membered aromatic heterocycle having 1, 2 or 3 N atoms and optionally an O atom or S atom, where this heterocycle is linked to the N atom of the skeleton via the ring C atom, and is selected from the group consisting of pyridinyl, pyrimidinyl, pyrazolyl, thiazolyl, imidazolyl; pyrrolo[3,2-c]pyridinyl, pyrrolo[2,3-b]pyridinyl and quinolinyl; and where this heterocycle may be unsubstituted or substituted by one, two or three substituents, which are selected, independently of one another, from the group consisting of: Hal, A, Het², CN, —(CY₂)ₚ—OY, —(CY₂)ₚ—OZ, —(CY₂)ₚ—O-Het², —(CY₂)ₚ—O—(CY₂)ₜ-Het², —(CY₂)ₚ—O—(CY₂)ₜ—NYY, —(CY₂)ₚ—O—(CY₂)ₜ—OY, —(CY₂)ₚ—O—(CY₂)ₜ—POAA, —(CY₂)ₚ—NYY, —(CY₂)ₚ—COOY, —(CY₂)ₚ—CO—NYY, —(CY₂)ₚ—NY—COY, —SO₂—Het², CyA, —(CY₂)ₚ—O—(CY₂)ₜ—SO₂—Y, —(CY₂)ₚ—NY—SO₂—Y, and —(CY₂)ₚ—SO₂—Y,
Y denotes H or A,
Z denotes unbranched or branched alkenyl having 2, 3, 4, 5, 6, 7, 8, 9 or 10 C atoms, where, independently of one another, 1, 2, 3, 4, 5, 6 or 7 H atoms may be replaced by Hal,
CyA denotes cycloalkyl having 3, 4, 5, 6, 7 or 8 ring C atoms which is unsubstituted or mono- or polysubstituted, independently of one another, by Hal, A, CN, —(CY₂)ₚ—OY, —(CY₂)ₚ—NYY, —(CY₂)ₚ—COOY, —(CY₂)ₚ—CO—NYY and/or —(CY₂)ₚ—NY—COY,
Hal denotes F, Cl, Br or I, and
p denotes 0, 1, 2, 3, 4, 5 or 6,
t denotes 1, 2, 3, 4, 5 or 6,
and/or pharmaceutically acceptable salt thereof.
35. The compounds for use, method of treatment or use according to item 34, wherein the ATM inhibitor is 3-Fluoro-4-[7-methoxy-3-methyl-8-(1-methyl-1 H-pyrazol-4-yl)-2-oxo-2,3-dihydroimidazo[4,5-c]-quinolin-1-yl]benzonitrile or a pharmaceutically acceptable salt thereof.
36. The compounds for use, method of treatment or use according to item 34, wherein the ATM inhibitor is 8-(1,3-Dimethyl-1 H-pyrazol-4-yl)-1-(3-fluoro-5-methoxy-pyridin-4-yl)-7-methoxy-3-methyl-1,3-dihydro-imidazo[4,5-c]quinolin-2-one or a pharmaceutically acceptable salt thereof.
37. The compounds for use, method of treatment or use according to item 36, wherein the ATM inhibitor is 8-(1,3-Dimethyl-1 H-pyrazol-4-yl)-1-(Sa)-(3-fluoro-5-methoxy-pyridin-4-yl)-7-methoxy-3-methyl-1,3-dihydro-imidazo[4,5-c]quinolin-2-one or a pharmaceutically acceptable salt thereof.
38. A PD-1 inhibitor, a TGFβ inhibitor and an ATM inhibitor for use in a method of treating a cancer in a subject, wherein the method comprises administering the PD-1 inhibitor, the TGFβ inhibitor and the ATM inhibitor to the subject in combination with radiotherapy;
   wherein the PD-1 and the TGFβ inhibitor are fused and the amino acid sequence of the fusion molecule is identical to the amino acid sequence of bintrafusp alfa; and
   wherein the ATM inhibitor is 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, preferably 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one, or a pharmaceutically acceptable salt thereof.
39. An ATM inhibitor for use in a method of treating a cancer in a subject, wherein the method comprises administering the ATM inhibitor to the subject in combination with a PD-1 inhibitor, a TGFβ inhibitor, and radiotherapy;
   wherein the PD-1 and the TGFβ inhibitor are fused and the amino acid sequence of the fusion molecule is identical to the amino acid sequence of bintrafusp alfa; and
   wherein the ATM inhibitor is 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, preferably 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one, or a pharmaceutically acceptable salt thereof.
40. A PD-1 inhibitor and a TGFβ inhibitor for use in a method of treating a cancer in a subject, wherein the method comprises administering the PD-1 inhibitor and the TGFβ inhibitor to the subject in combination with an ATM inhibitor and radiotherapy; and
   wherein the PD-1 and the TGFβ inhibitor are fused and the amino acid sequence of the fusion molecule is identical to the amino acid sequence of bintrafusp alfa; and
   wherein the ATM inhibitor is 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, preferably 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one, or a pharmaceutically acceptable salt thereof.
41. A method of treating a cancer in a subject, wherein the method comprises administering a PD-1 inhibitor, a TGFβ inhibitor and an ATM inhibitor to the subject in combination with radiotherapy;
   wherein the PD-1 and the TGFβ inhibitor are fused and the amino acid sequence of the fusion molecule is identical to the amino acid sequence of bintrafusp alfa; and
   wherein the ATM inhibitor is 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, preferably 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one, or a pharmaceutically acceptable salt thereof.
42. Use of a PD-1 inhibitor, a TGFβ inhibitor and an ATM inhibitor for the manufacture of a medicament for a method of treating a cancer in a subject, wherein the method comprises administering the PD-1 inhibitor, the TGFβ inhibitor and the ATM inhibitor to the subject in combination with radiotherapy;
   wherein the PD-1 and the TGFβ inhibitor are fused and the amino acid sequence of the fusion molecule is identical to the amino acid sequence of bintrafusp alfa; and
   wherein the ATM inhibitor is 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, preferably 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one, or a pharmaceutically acceptable salt thereof.
43. Use of an ATM inhibitor for the manufacture of a medicament for a method of treating a cancer in a subject, wherein the method comprises administering the ATM inhibitor to the subject in combination with a PD-1 inhibitor, a TGFβ inhibitor, and radiotherapy;
   wherein the PD-1 and the TGFβ inhibitor are fused and the amino acid sequence of the fusion molecule is identical to the amino acid sequence of bintrafusp alfa; and
   wherein the ATM inhibitor is 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, preferably 8-(1,3-Dimethyl-1 H-pyrazol-4-yl)-1-(Sa)-(3-fluoro-5-methoxy-pyridin-4-yl)-7-methoxy-3-methyl-1,3-dihydro-imidazo[4,5-c]quinolin-2-one, or a pharmaceutically acceptable salt thereof.
44. Use of a PD-1 inhibitor and a TGFβ inhibitor for the manufacture of a medicament for a method of treating a cancer in a subject, wherein the method comprises administering the PD-1 inhibitor and the TGFβ inhibitor to the subject in combination with an ATM inhibitor and radiotherapy;
   wherein the PD-1 and the TGFβ inhibitor are fused and the amino acid sequence of the fusion molecule is identical to the amino acid sequence of bintrafusp alfa; and
   wherein the ATM inhibitor is 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, preferably 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one, or a pharmaceutically acceptable salt thereof.
45. The compounds for use, method of treatment or use according to any one of items 1 to 44, wherein the cancer is selected from the group consisting of carcinoma, lymphoma, leukemia, blastoma, and sarcoma.
46. The compounds for use, method of treatment or use according to any one of items 1 to 45, wherein the cancer is selected from the group consisting of squamous cell carcinoma, myeloma, small-cell lung cancer, non-small cell lung cancer, glioma, Hodgkin's lymphoma, non-Hodgkin's lymphoma, acute myeloid leukemia, multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma, cervical cancer, brain cancer, stomach cancer, bladder cancer, hepatoma, breast cancer, colon carcinoma, biliary tract cancer, and head and neck cancer.
47. The compounds for use, method of treatment or use according to any one of items 1 to 46, wherein the PD-1 inhibitor, TGFβ inhibitor, ATM inhibitor and radiotherapy are administered in a first-line treatment of the cancer.
48. The compounds for use, method of treatment or use according to any one of items 1 to 46, wherein the subject underwent at least one round of prior cancer therapy.
49. The compounds for use, method of treatment or use according item 48, wherein the cancer was resistant or became resistant to prior therapy.
50. The compounds for use, method of treatment or use according to any one of items 1 to 46, wherein the PD-1 inhibitor, TGFβ inhibitor, ATM inhibitor and radiotherapy are administered in a second-line or higher treatment of the cancer.
51. The compounds for use, method of treatment or use according to item 50, wherein the cancer is selected from the group consisting of pre-treated relapsing metastatic NSCLC, unresectable locally advanced NSCLC, pre-treated SCLC ED, SCLC unsuitable for systemic treatment, pre-treated relapsing or metastatic SCCHN, recurrent SCCHN eligible for re-irradiation, and pre-treated microsatellite status instable low (MSI-L) or microsatellite status stable (MSS) metastatic colorectal cancer (mCRC).
52. The compounds for use, method of treatment or use according to any one of items 1 to 51, wherein the radiotherapy comprises about 35-70 Gy/20-35 fractions.
53. The compounds for use, method of treatment or use according to any one of items 1 to 52, wherein the radiotherapy is selected from a treatment given with electrons, photons, protons, alfa-emitters, other ions, radio-nucleotides, boron capture neutrons and combinations thereof.
54. The compounds for use, method of treatment or use according to any one of items 1 to 53, wherein the PD-L1 inhibitor and the TGFβ inhibitor are fused and administered via intravenous infusion.
55. The compounds for use, method of treatment or use according to any one of items 1 to 54, wherein the PD-L1 inhibitor and the TGFβ inhibitor are fused and administered at a dose of about 1200 mg, 1800 mg or 2400 mg.
56. The compounds for use, method of treatment or use according to any one of items 1 to 54, wherein the PD-L1 inhibitor and the TGFβ inhibitor are fused and administered once every two weeks, preferably, 1200 mg, or once every three weeks, preferably, 1800 mg or 2400 mg.
57. The compounds for use, method of treatment or use according to any one of items 1 to 56, wherein the ATM inhibitor is administered orally.
58. The compounds for use, method of treatment or use according to any one of items 1 to 57, wherein the ATM inhibitor is administered once daily (QD) or twice daily (BID).
59. The compounds for use, method of treatment or use according to any one of items 1 to 58, wherein the ATM inhibitor is administered at a dose of about 20 to 500, preferably 25 to 400 mg per day.
60. The compounds for use, method of treatment or use according to any one of items 1 to 59, wherein the method comprises a lead phase, optionally followed by a maintenance phase after completion of the lead phase.
61. The compounds for use, method of treatment or use according to item 60, wherein the PD-1 inhibitor, TGFβ inhibitor, ATM inhibitor and radiotherapy are administered concurrently in either the lead or maintenance phase and optionally non-concurrently in the other phase, or the PD-1 inhibitor, TGFβ inhibitor, ATM inhibitor and radiotherapy are administered non-concurrently in the lead and maintenance phase, or two or more of the PD-1 inhibitor, TGFβ inhibitor, ATM inhibitor and radiotherapy are administered concurrently and the others non-concurrently in the lead and maintenance phase.
62. The compounds for use, method of treatment or use according to item 61, wherein the concurrent administration occurs sequentially in either order or substantially simultaneously.
63. The compounds for use, method of treatment or use according to any one of items 60 to 62, wherein the maintenance phase comprises administration of the PD-1 inhibitor alone or concurrently with the ATM inhibitor, the TGFβ inhibitor and/or radiotherapy, or none of them.
64. The compounds for use, method of treatment or use according to any one of items 60 to 62, wherein the PD-1 inhibitor and TGFβ inhibitor are fused and the maintenance phase comprises administration of the fused PD-1 inhibitor and TGFβ inhibitor alone or concurrently with the ATM inhibitor and/or radiotherapy, or none of them.
65. The compounds for use, method of treatment or use according to any one of items 60 to 64, wherein the lead phase comprises the concurrent administration of the PD-1 inhibitor, TGFβ inhibitor, ATM inhibitor and radiotherapy.
66. The compounds for use, method of treatment or use according to any one of items 1 to 65, wherein the cancer is selected based on PD-L1 expression in samples taken from the subject.
67. A pharmaceutical composition comprising a PD-1 inhibitor, a TGFβ inhibitor, an ATM inhibitor and at least a pharmaceutically acceptable excipient or adjuvant.
68. The pharmaceutical composition according to item 67, wherein the PD-1 inhibitor and the TGFβ inhibitor are fused.
69. The pharmaceutical composition according to item 67 or 68, wherein the ATM inhibitor is 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one, preferably 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one, or a pharmaceutically acceptable salt thereof.
70. The pharmaceutical composition according to any one of items 67 to 69 for use in therapy, preferably for use in treating cancer.
71. The pharmaceutical composition for use according to item 70, wherein the therapy further comprises radiotherapy.
72. A kit comprising a PD-1 inhibitor and a package insert comprising instructions for using the PD-1 inhibitor in combination with an ATM inhibitor, a TGFβ inhibitor and radiotherapy to treat or delay progression of a cancer in a subject.
73. A kit comprising an ATM inhibitor and a package insert comprising instructions for using the ATM inhibitor in combination with a PD-1 inhibitor, a TGFβ inhibitor and radiotherapy to treat or delay progression of a cancer in a subject.
74. A kit comprising a TGFβ inhibitor and a package insert comprising instructions for using the TGFβ inhibitor in combination with a PD-1 inhibitor, an ATM inhibitor and radiotherapy to treat or delay progression of a cancer in a subject.
75. A kit comprising anti-PD-L1/TGFβ Trap and a package insert comprising instructions for using the anti-PD-L1/TGFβ Trap in combination with an ATM inhibitor and radiotherapy to treat or delay progression of a cancer in a subject.
76. The kit according to any one of items 72 to 75, wherein the instructions state that the medicaments are intended for use in treating a subject having a cancer that tests positive for PD-L1 expression.
77. A method for advertising a PD-1 inhibitor, a TGFβ inhibitor, an ATM inhibitor and radiotherapy comprising promoting, to a target audience, the use of the combination for treating a subject with a cancer, preferably a cancer selected based on PD-L1 expression in samples taken from the subject.

All the references cited herein are incorporated by reference in the disclosure of the invention hereby.

It is to be understood that this invention is not limited to the particular molecules, pharmaceutical compositions, uses and methods described herein, as such matter can, of course, vary. It is also to be understood that the terminology used herein is for the purpose of describing particular embodiments only and is not intended to limit the scope of the present invention, which is only defined by the appended claims. The techniques that are essential according to the invention are described in detail in the specification. Other techniques which are not described in detail correspond to known standard methods that are well known to a person skilled in the art, or the techniques are described in more detail in cited references, patent applications or standard literature. Provided that no other hints in the application are given, they are used as examples only, they are not considered to be essential according to the invention, but they can be replaced by other suitable tools and biological materials.

Although methods and materials similar or equivalent to those described herein can be used in the practice or testing of the present invention, suitable examples are described below. Within the examples, standard reagents and buffers that are free from contaminating activities (whenever practical) are used. The examples are particularly to be construed such that they are not limited to the explicitly demonstrated combinations of features, but the exemplified features may be unrestrictedly combined again provided that the technical problem of the invention is solved. Similarly, the features of any claim can be combined with the features of one or more other claims. The present invention having been described in summary and in detail, is illustrated and not limited by the following examples.

### EXAMPLES

### Example 1: Evaluation of the Antitumor Activity of Bintrafusp Alfa and Radiotherapy (RT) with the ATM Inhibitor, Compound A, in the 4T1 Mammary Tumor Model

The antitumor activity of bintrafusp alfa and radiotherapy (RT) with the ATM inhibitor, Compound A, was evaluated in the 4T1 mammary tumor model.

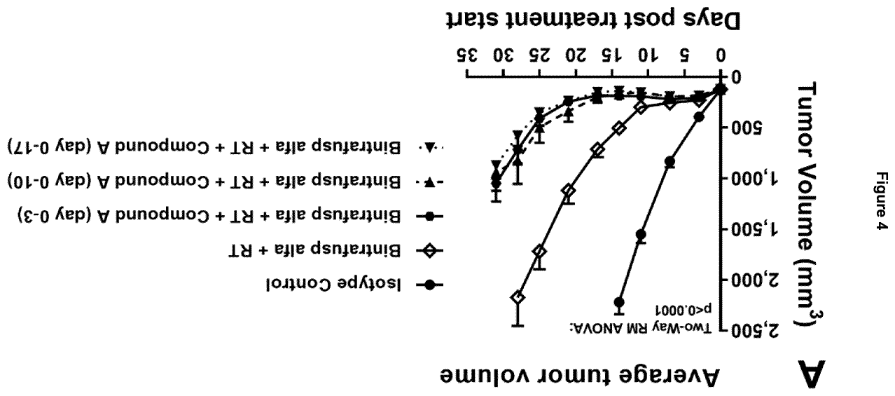

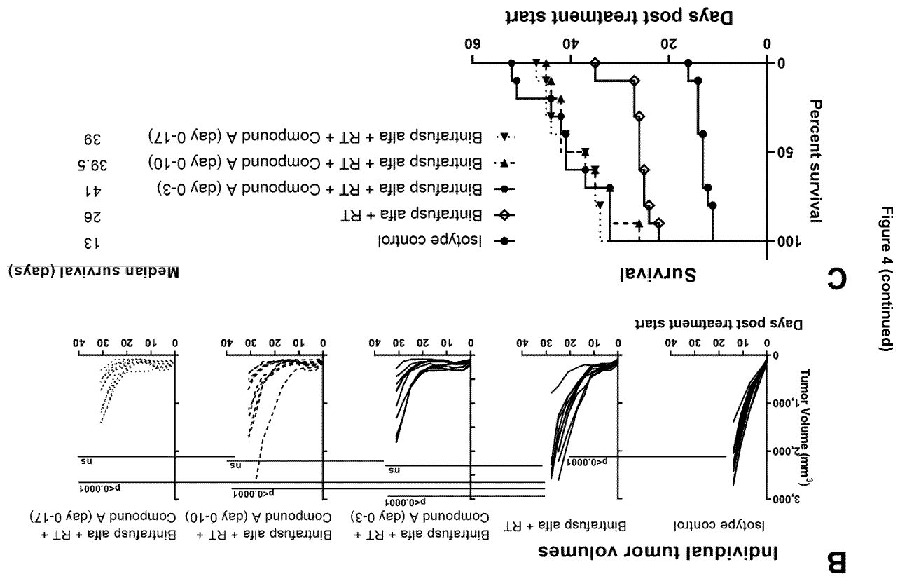

Dual combination therapy with bintrafusp alfa and RT significantly inhibited tumor growth relative to the isotype control, which lacked the PD-L1 binding function while still being capable of binding TGF-β(p<0.0001, day 13). The combination of bintrafusp alfa and RT with Compound A further enhanced antitumor activity relative to bintrafusp alfa and RT dual combination therapy (p<0.0001, day 13 and day 28) (FIG. 3A-B). In addition, median survival was significantly prolonged with the combination of bintrafusp alfa, RT, and Compound A (34 days) compared with bintrafusp alfa and RT dual combination therapy (26 days, p<0.0001) or isotype control (13 days, p<0.0001) (FIG. 3C).

### Example 2: Evaluating the Influence of the Dosing Schedule of Compound A on the Triple Combination of Bintrafusp Alfa, RT, and Compound A in the 4T1 Mammary Tumor Model

The effect of different dosing schedules of Compound A on the antitumor activity of the triple combination therapy was evaluated in the 4T1 mammary tumor model.

Similar to Example 1, dual combination therapy with bintrafusp alfa and RT significantly inhibited tumor growth relative to isotype control (p<0.0001, day 14), but the combination of bintrafusp alfa, RT, and Compound A further enhanced antitumor activity relative to bintrafusp alfa and RT dual combination therapy, regardless of the dosing schedule of Compound A (p<0.0001, day 14 and day 28) (FIG. 4A-B). In fact, there was no significant difference between the antitumor activity of the triple combination therapy when Compound A was given on days 0-3, days 0-10, or days 0-17. The median survival was also not significantly different between triple combination therapy dosed with Compound A on days 0-3 (41 days), days 0-10 (39.5 days), or days 0-17 (39 days). However, triple combination therapy, regardless of Compound A dosing schedule, did significantly prolong median survival relative to bintrafusp alfa and RT dual combination therapy (26 days, p<0.0001) or isotype control (13 days, p<0.0001) (FIG. 4C).

### Example 3: Evaluating the Triple Combination of Bintrafusp Alfa, RT, and Compound A in Comparison with Dual and Monotherapy Controls in the 4T1 Mammary Tumor Model

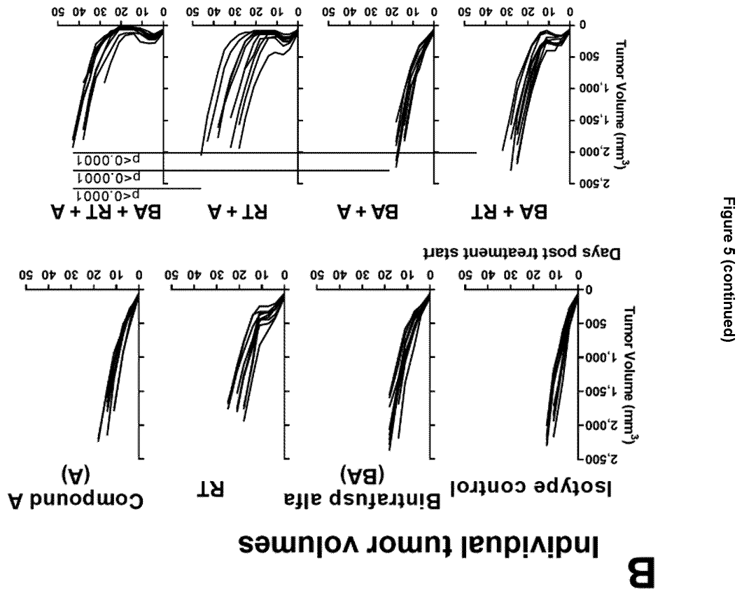

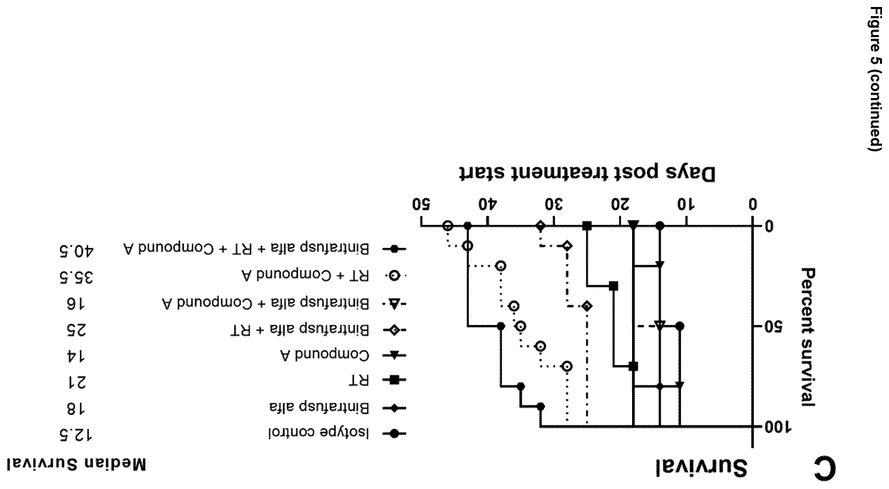

Example 3 evaluated the triple combination of bintrafusp alfa, RT, and Compound A in comparison with dual and monotherapy controls in the 4T1 mammary tumor model. Relative to isotype control, tumor growth was significantly inhibited with bintrafusp alfa (p<0.0001, day 14), RT (p<0.0001, day 14), or Compound A (p<0.0001, day 14) monotherapies (FIG. 5A-B). Although bintrafusp alfa and Compound A dual combination therapy did not further enhance antitumor activity relative to bintrafusp alfa and Compound A monotherapies (p>0.05, day 18), bintrafusp alfa and RT dual combination therapy enhanced antitumor activity relative to bintrafusp alfa (p<0.0001, day 18) and RT (p<0.0001, day 18) monotherapies, and RT and Compound A dual combination therapy enhanced antitumor activity relative to RT (p<0.0001, day 18) and Compound A (p<0.0001, day 18) monotherapies (FIG. 5A-B). Furthermore, the combination of bintrafusp alfa, RT, and Compound A significantly enhanced antitumor activity relative to bintrafusp alfa+RT (p<0.0001, day 28), bintrafusp alfa+Compound A (p<0.0001, day 18), and RT+Compound A (p<0.0001, day 28). Triple combination therapy also prolonged median survival (40.5 days) relative to monotherapies and dual combination therapies, though not significantly relative to RT+Compound A (35.5 days) (FIG. 5C).

### Example 4: Evaluating the Triple Combination of Bintrafusp Alfa, RT, and Compound A in Comparison with the Dual Combination of Bintrafusp Alfa and RT in the 4T1 Mammary Tumor Model

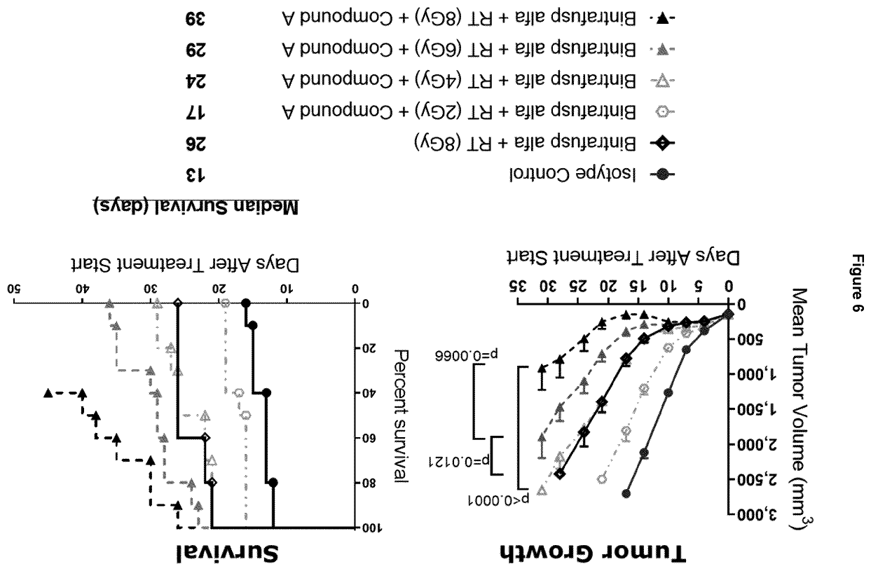

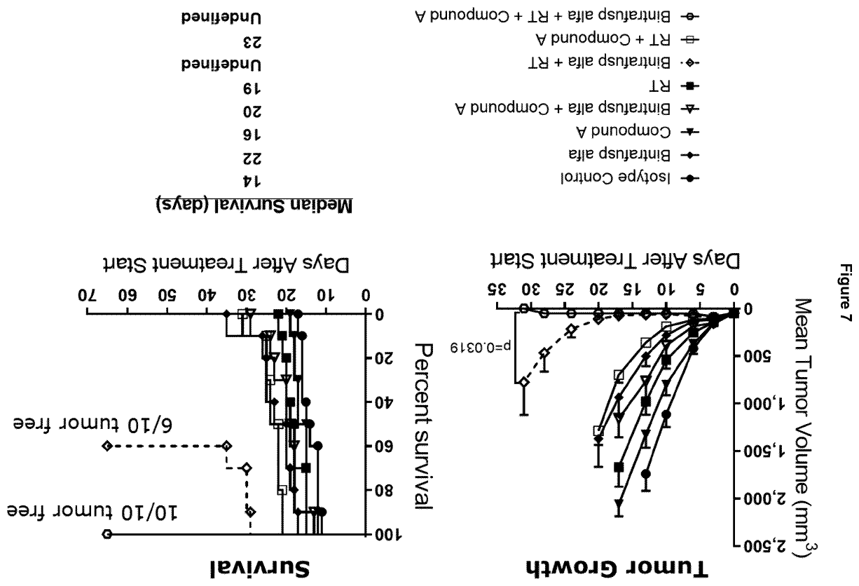

The 4T1 murine breast cancer cell line was injected into Balb/c mice and tumor bearing animals were treated with bintrafusp alfa+RT (8Gy, QDx4) or bintrafusp alfa+Compound A plus various doses of RT (2Gy, QDx4; 4Gy, QDx4; 6Gy, QDx4; 8Gy, QDx4). In this model, the combination of bintrafusp alfa+Compound A+8Gy x4 RT showed the greatest inhibition of tumor growth relative to isotype control (p<0.0001, day 14), bintrafusp alfa+8Gy, QDx4 RT (p<0.0001, day 21) or bintrafusp alfa+Compound A+6Gy, QDx4 RT (p=0.0066). The bintrafusp alfa+Compound A+8Gy, QDx4 RT group showed the greatest overall median survival (median survival=39 days) verses bintrafusp alfa+8Gy, QDx4 RT (median survival=26 days; p<0.0001) or bintrafusp alfa+Compound A+6Gy, QDx4 RT (median survival=29 days; p=0.0038). The triple combination therapy of bintrafusp alfa+Compound A+4Gy, QDx4 RT showed equivalent efficacy and survival to the dual combination therapy of bintrafusp alfa+8Gy, QDx4 RT (p>0.9999, tumor growth at day 21; p=0.5124, median survival). The triple combination therapy of bintrafusp alfa+Compound A+6Gy, QDx4 RT showed superior efficacy and survival to the dual combination therapy of bintrafusp alfa+8Gy, QDx4 RT (p>0.0.0121, tumor growth at day 21; p=0.0017, median survival) (FIG. 6).

### Example 5: Evaluating the Triple Combination of Bintrafusp Alfa, RT, and Compound A in Comparison with Dual and Monotherapy Controls in the MC38 Murine Colon Carcinoma Model

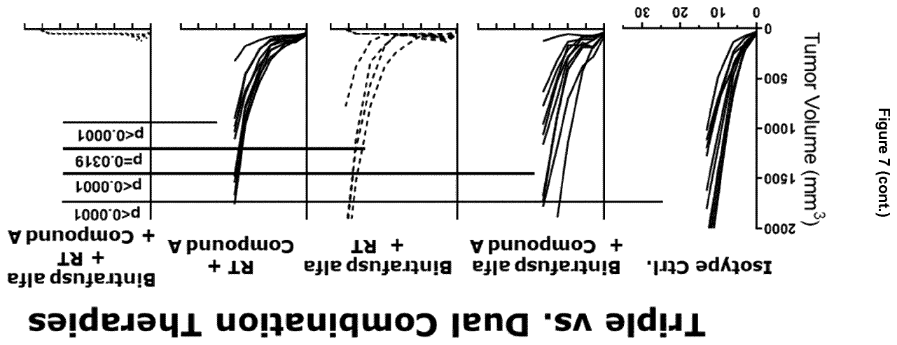

In an intramuscular MC38 murine colon carcinoma model, the combination of bintrafusp alfa+Compound A+RT showed significantly greater tumor growth inhibition relative to isotype control (p<0.0001, day 13), bintrafusp alfa+Compound A (p<0.0001, day 17), bintrafusp alfa+RT (p<0.0319, day 31) or Compound A+RT (p<0.0001, day 20). The bintrafusp alfa+Compound A+RT treatment resulted in 10 of 10 mice showing tumor free survival at day 65, whereas the bintrafusp alfa+RT dual combination resulted in 6 of 10 mice showing tumor free survival. No other treatment group in the study produced showed a complete tumor regression and long term tumor free survival (FIG. 7).

### Materials and Methods of Examples 1-5

Cell Lines

4T1 murine breast cancer cells were obtained from the American Type Culture Collection (ATCC). 4T1 cells were cultured in RPM11640 medium supplemented with 10% FBS, 2 mM L-glutamine, 10 mM HEPES, 1 mM sodium pyruvate, 4500 mg/L glucose, and 1500 mg/L sodium bicarbonate. The MC38 murine colon carcinoma cell line was cultured in DMEM medium supplemented with 10% FBS, 4500 mg/L glucose and 2 mM L-glutamine. Cells were passaged before in vivo implantation and adherent cells were harvested with TrypLE Express (Gibco) or 0.25% trypsin.

Mice

BALB/c and C57BL/6 mice were obtained from Charles River Laboratories. All mice used for experiments were 6- to 12-week-old females. Mice were housed with ad libitum access to food and water in pathogen-free facilities.

Murine Tumor Models

4T1 Tumor Model

For efficacy and survival studies of Examples 1 to 3, 4T1 cells, 0.5×10⁵, were inoculated intramuscularly (i.m.) in the thigh of BALB/c mice on day −7. Treatment was initiated 7 days later on day 0, and mice were sacrificed when tumor volumes reached ˜2000 mm³.

For the efficacy study of Examples 4 and 5, 0.5×10⁵ 4T1 cells were inoculated intramuscularly (i.m.) into the right thigh of BALB/c mice on day −7. Treatment was initiated on day 0 when average tumor volume reached ˜150 mm³. Mice were sacrificed when tumor volumes reached ˜2500 mm³.

MC38 Tumor Model

For the efficacy study, 0.25×10⁵ MC38 cells were inoculated intramuscularly into the right thigh of C57BL/6 mice on day −7. Treatment was initiated on day 0 when average tumor volume reached ˜50 mm³. Mice were sacrificed when tumor volumes reached ˜2500 mm³.

Treatment

For all studies, mice were randomized into treatment groups on the day of treatment initiation (day 0).

Bintrafusp Alfa and Isotype Control

In tumor-bearing mice, bintrafusp alfa (492 μg or 164 μg) or isotype control (400 μg or 133 μg) were administered with an intravenous injection (i.v.) in 0.2 mL PBS. Exact dose and treatment schedules for each experiment are listed in the figure legends. The isotype control of bintrafusp alfa is a mutated version of bintrafusp alfa, which completely lacks PD-L1 binding (while still being capable of binding TGF-β).

Radiotherapy (RT)

To deliver the 2, 4, 6 or 8 Gy/day dose of RT, a collimator device with lead shielding was used to localize delivery to the tumor-bearing thigh of mice. This region was irradiated by timed exposure to a Cesium-137 gamma irradiator (GammaCell 40 Exactor, MDS Nordion, Ottawa, ON, Canada). RT was given once per day for four days (days 0-3). Exact dose and treatment schedules for each experiment are listed in the figure legends.

Compound A and Vehicle Control

Compound A, specifically Compound A1, was administered via oral gavage (p.o.) at 100 mg/kg (10 μL/g). The vehicle control of Compound A, 0.5% Methocel K4M Premium+0.25% Tween 20 in water, was administered p.o. (10 μL/g). Exact dose and treatment schedules for each experiment are listed in the figure legends. Tumor-bearing mice were treated with 1 dose per day for 4, 5, 11, or 18 days (days 0-3, 0-4, 0-10, or 0-17).

Tumor Growth and Survival

Tumor sizes were measured twice per week with digital calipers and recorded automatically using WinWedge software in Examples 1-3 or StudyLog software in Examples 4 and 5. Tumor volumes were calculated with the following formula: tumor volume (mm³)=tumor length×width×height×0.5236. Tumor growth inhibition (TGI) was calculated with the following formula: TGI (%)=[1−(Ti−T0)/(Vi−V0)]×100, where Ti is the average tumor volume (mm³) of a treatment group on a given day, TO is the average tumor volume of the treatment group on the first day of treatment, Vi is the average tumor volume of the vehicle control group on the same day as Ti, and V0 is the average tumor volume of the vehicle control group on the first day of treatment.

To compare the percentage survival between different treatment groups, Kaplan-Meier survival curves were generated. Body weight was measured twice weekly and mice were sacrificed when their tumor volume exceeded 2,000 mm³.

Statistical Analyses

Statistical analyses were performed using GraphPad Prism Software, version 8.0 or 8.0.1. Tumor volume data are presented graphically as mean±SEM by symbols or as individual mice by lines. To assess differences in tumor volumes between treatment groups two-way analysis of variance (ANOVA) was performed followed by Tukey's or Sidak's multiple comparison test. A Kaplan-Meier plot was generated to show survival by treatment group and significance was assessed by log-rank (Mantel-Cox) test.

SEQUENCE LISTINGS

| SEQ | | |
|---|---|---|
| ID NO. | Sequence | Description |
| 1 | SYIMM | Bintrafusp alfa CDRH1 |
| 2 | SIYPSGGITFYADTVKG | Bintrafusp alfa CDRH2 |
| 3 | IKLGTVTTVDY | Bintrafusp alfa CDRH3 |
| 4 | TGTSSDVGGYNYVS | Bintrafusp alfa CDRL1 |
| 5 | DVSNRPS | Bintrafusp alfa CDRL2 |
| 6 | SSYTSSSTRV | Bintrafusp alfa CDRL3 |
| 7 | QSALTQPASVSGSPGQSITISCTGTSSDVGGYNYVSWYQQHP | Bintrafusp alfa light chain |
| | GKAPKLMIYDVSNRPSGVSNRFSGSKSGNTASLTISGLQAEDE | |
| | ADYYCSSYTSSSTRVFGTGTKVTVLGQPKANPTVTLFPPSSEE | |
| | LQANKATLVCLISDFYPGAVTVAWKADGSPVKAGVETTKPSK | |
| | QSNNKYAASSYLSLTPEQWKSHRSYSCQVTHEGSTVEKTVAP | |
| | TECS | |
| 8 | EVQLLESGGGLVQPGGSLRLSCAASGFTFSSYIMMWVRQAP | Bintrafusp alfa heavy |
| | GKGLEWVSSIYPSGGITFYADTVKGRFTISRDNSKNTLYLQMN | chain |
| | SLRAEDTAVYYCARIKLGTVTTVDYWGQGTLVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKRVEPKSCDKTHTCPPCPAPELLGGPSVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPIEKTI | |
| | SKAKGQPREPQVYTLPPSREEMTKNQVSLTCLVKGFYPSDIA | |
| | VEWESNGQPENNYKTTPPVLDSDGSFFLYSKLTVDKSRWQQ | |
| | GNVFSCSVMHEALHNHYTQKSLSLSPGAGGGGSGGGGSGG | |
| | GGSGGGGSGIPPHVQKSVNNDMIVTDNNGAVKFPQLCKFCD | |
| | VRFSTCDNQKSCMSNCSITSICEKPQEVCVAVWRKNDENITLE | |
| | TVCHDPKLPYHDFILEDAASPKCIMKEKKKPGETFFMCSCSSD | |
| | ECNDNHFSEEYNTSNPD | |
| 9 | MGRGLLRGLWPLHIVLWTRIASTIPPHVQKSDVEMEAQKDEIIC | TGFβRII isoform A |
| | PSCNRTAHPLRHINNDMIVTDNNGAVKFPQLCKFCDVRFSTC | |
| | DNQKSCMSNCSITSICEKPQEVCVAVWRKNDENITLETVCHDP | |
| | KLPYHDFILEDAASPKCIMKEKKKPGETFFMCSCSSDECNDNII | |
| | FSEEYNTSNPDLLLVIFQVTGISLLPPLGVAISVIIIFYCYRVNRQ | |
| | QKLSSTWETGKTRKLMEFSEHCAIILEDDRSDISSTCANNINHN | |
| | TELLPIELDTLVGKGRFAEVYKAKLKQNTSEQFETVAVKIFPYE | |
| | EYASWKTEKDIFSDINLKHENILQFLTAEERKTELGKQYWLITA | |
| | FHAKGNLQEYLTRHVISWEDLRKLGSSLARGIAHLHSDHTPCG | |
| | RPKMPIVHRDLKSSNILVKNDLTCCLCDFGLSLRLDPTLSVDDL | |
| | ANSGQVGTARYMAPEVLESRMNLENVESFKQTDVYSMALVL | |
| | WEMTSRCNAVGEVKDYEPPFGSKVREHPCVESMKDNVLRDR | |
| | GRPEIPSFWLNHQGIQMVCETLTECWDHDPEARLTAQCVAER | |
| | FSELEHLDRLSGRSCSEEKIPEDGSLNTTK | |
| 10 | MGRGLLRGLWPLHIVLWTRIASTIPPHVQKSVNNDMIVTDNNG | TGFβRII isoform B |
| | AVKFPQLCKFCDVRFSTCDNQKSCMSNCSITSICEKPQEVCVA | |
| | VWRKNDENITLETVCHDPKLPYHDFILEDAASPKCIMKEKKKP | |
| | GETFFMCSCSSDECNDNIIFSEEYNTSNPDLLLVIFQVTGISLLP | |
| | PLGVAISVIIIFYCYRVNRQQKLSSTWETGKTRKLMEFSEHCAII | |
| | LEDDRSDISSTCANNINHNTELLPIELDTLVGKGRFAEVYKAKL | |
| | KQNTSEQFETVAVKIFPYEEYASWKTEKDIFSDINLKHENILQFL | |
| | TAEERKTELGKQYWLITAFHAKGNLQEYLTRHVISWEDLRKLG | |
| | SSLARGIAHLHSDHTPCGRPKMPIVHRDLKSSNILVKNDLTCCL | |
| | CDFGLSLRLDPTLSVDDLANSGQVGTARYMAPEVLESRMNLE | |
| | NVESFKQTDVYSMALVLWEMTSRCNAVGEVKDYEPPFGSKV | |
| | REHPCVESMKDNVLRDRGRPEIPSFWLNHQGIQMVCETLTEC | |
| | WDHDPEARLTAQCVAERFSELEHLDRLSGRSCSEEKIPEDGS | |
| | LNTTK | |
| 11 | IPPHVQKSVNNDMIVTDNNGAVKFPQLCKFCDVRFSTCDNQK | TGFβRII extracellular |
| | SCMSNCSITSICEKPQEVCVAVWRKNDENITLETVCHDPKLPY | domain fragment |
| | HDFILEDAASPKCIMKEKKKPGETFFMCSCSSDECNDNIIFSEE | |
| | YNTSNPD | |
| 12 | GAVKFPQLCKFCDVRFSTCDNQKSCMSNCSITSICEKPQEVC | TGFβRII extracellular |
| | VAVWRKNDENITLETVCHDPKLPYHDFILEDAASPKCIMKEKK | domain fragment |
| | KPGETFFMCSCSSDECNDNIIFSEEYNTSNPD | |
| 13 | VKFPQLCKFCDVRFSTCDNQKSCMSNCSITSICEKPQEVCVAV | TGFβRII extracellular |
| | WRKNDENITLETVCHDPKLPYHDFILEDAASPKCIMKEKKKPG | domain fragment |
| | ETFFMCSCSSDECNDNIIFSEEYNTSNPD | |
| 14 | QVQLVQSGAEVKKPGASVKVSCKASGYTFTSYWMHWVRQA | Anti-PD-L1 antibody |
| | PGQGLEWMGRIGPNSGFTSYNEKFKNRVTMTRDTSTSTVYM | heavy chain |
| | ELSSLRSEDTAVYYCARGGSSYDYFDYWGQGTTVTVSSASTK | |
| | GPSVFPLAPCSRSTSESTAALGCLVKDYFPEPVTVSWNSGAL | |
| | TSGVHTFPAVLQSSGLYSLSSVVTVPSSSLGTKTYTCNVDHKP | |
| | SNTKVDKRVESKYGPPCPPCPAPEAAGGPSVFLFPPKPKDTL | |
| | MISRTPEVTCVVVDVSQEDPEVQFNWYVDGVEVHNAKTKPRE | |
| | EQFNSTYRVVSVLTVLHQDWLNGKEYKCKVSNKGLPSSIEKTI | |
| | SKAKGQPREPQVYTLPPSQEEMTKNQVSLTCLVKGFYPSDIA | |
| | VEWESNGQPENNYKTTPPVLDSDGSFFLYSRLTVDKSRWQE | |
| | GNVFSCSVMHEALHNHYTQKSLSLSLGA | |
| 15 | DIVLTQSPASLAVSPGQRATITCRASESVSIHGTHLMHWYQQK | Anti-PD-L1 antibody light |
| | PGQPPKLLIYAASNLESGVPARFSGSGSGTDFTLTINPVEAED | chain |
| | TANYYCQQSFEDPLTFGQGTKLEIKRTVAAPSVFIFPPSDEQL | |
| | KSGTASVVCLLNNFYPREAKVQWKVDNALQSGNSQESVTEQ | |
| | DSKDSTYSLSSTLTLSKADYEKHKVYACEVTHQGLSSPVTKSF | |
| | NRGEC | |
| 16 | EVQLLESGGGLVQPGGSLRLSCAASGFTFSSYIMMWVRQAP | Anti-PD-L1 antibody |
| | GKGLEWVSSIYPSGGITFYADTVKGRFTISRDNSKNTLYLQMN | heavy chain |
| | SLRAEDTAVYYCARIKLGTVTTVDYWGQGTLVTVSSASTKGPS | |
| | VFPLAPSSKSTSGGTAALGCLVKDYFPEPVTVSWNSGALTSG | |
| | VHTFPAVLQSSGLYSLSSVVTVPSSSLGTQTYICNVNHKPSNT | |
| | KVDKRVEPKSCDKTHTCPPCPAPELLGGPSVFLFPPKPKDTL | |
| | MISRTPEVTCVVVDVSHEDPEVKFNWYVDGVEVHNAKTKPRE | |
| | EQYNSTYRVVSVLTVLHQDWLNGKEYKCKVSNKALPAPIEKTI | |
| | SKAKGQPREPQVYTLPPSREEMTKNQVSLTCLVKGFYPSDIA | |
| | VEWESNGQPENNYKTTPPVLDSDGSFFLYSKLTVDKSRWQQ | |
| | GNVFSCSVMHEALHNHYTQKSLSLSPGA | |
| 17 | QVQLVQSGAEVKKPGASVKVSCKASGYTFTSYWMHWVRQA | Anti-PD-L1:TGFβRII |
| | PGQGLEWMGRIGPNSGFTSYNEKFKNRVTMTRDTSTSTVYM | fusion protein heavy chain |
| | ELSSLRSEDTAVYYCARGGSSYDYFDYWGQGTTVTVSSASTK | as disclosed in WO |
| | GPSVFPLAPCSRSTSESTAALGCLVKDYFPEPVTVSWNSGAL | 2018/205985 |
| | TSGVHTFPAVLQSSGLYSLSSVVTVPSSSLGTKTYTCNVDHKP | |
| | SNTKVDKRVESKYGPPCPPCPAPEAAGGPSVFLFPPKPKDTL | |
| | MISRTPEVTCVVVDVSQEDPEVQFNWYVDGVEVHNAKTKPRE | |
| | EQFNSTYRVVSVLTVLHQDWLNGKEYKCKVSNKGLPSSIEKTI | |
| | SKAKGQPREPQVYTLPPSQEEMTKNQVSLTCLVKGFYPSDIA | |
| | VEWESNGQPENNYKTTPPVLDSDGSFFLYSRLTVDKSRWQE | |
| | GNVFSCSVMHEALHNHYTQKSLSLSLGAGGGGSGGGGSGG | |
| | GGSGGGGSGGAVKFPQLCKFCDVRFSTCDNQKSCMSNCSIT | |
| | SICEKPQEVCVAVWRKNDENITLETVCHDPKLPYHDFILEDAA | |
| | SPKCIMKEKKKPGETFFMCSCSSDECNDNIIFSEEYNTSNPD | |
| 18 | QVQLVQSGAEVKKPGASVKVSCKASGYTFTSYWMHWVRQA | Anti-PD-L1:TGFβRII |
| | PGQGLEWMGRIGPNSGFTSYNEKFKNRVTMTRDTSTSTVYM | fusion protein heavy chain |
| | ELSSLRSEDTAVYYCARGGSSYDYFDYWGQGTTVTVSSASTK | as disclosed in WO |
| | GPSVFPLAPCSRSTSESTAALGCLVKDYFPEPVTVSWNSGAL | 2018/205985 |
| | TSGVHTFPAVLQSSGLYSLSSVVTVPSSSLGTKTYTCNVDHKP | |
| | SNTKVDKRVESKYGPPCPPCPAPEAAGGPSVFLFPPKPKDTL | |
| | MISRTPEVTCVVVDVSQEDPEVQFNWYVDGVEVHNAKTKPRE | |
| | EQFNSTYRVVSVLTVLHQDWLNGKEYKCKVSNKGLPSSIEKTI | |
| | SKAKGQPREPQVYTLPPSQEEMTKNQVSLTCLVKGFYPSDIA | |
| | VEWESNGQPENNYKTTPPVLDSDGSFFLYSRLTVDKSRWQE | |
| | GNVFSCSVMHEALHNHYTQKSLSLSLGAGGGGSGGGGSGG | |
| | GGSGGGGSGGGGSGVKFPQLCKFCDVRFSTCDNQKSCMSN | |
| | CSITSICEKPQEVCVAVWRKNDENITLETVCHDPKLPYHDFILE | |
| | DAASPKCIMKEKKKPGETFFMCSCSSDECNDNIIFSEEYNTSN | |
| | PD | |
| 19 | SYWMH | CDRH1 ofanti-PD-L1 |
| | | antibody as disclosed in |
| | | WO 2018/205985 |
| 20 | RIX1PNSGX2TSYNEKFKN, wherein X1 is H or G and | CDRH2 ofanti-PD-L1 |
| | wherein X2 is G or F | antibody as disclosed in |
| | | WO 2018/205985 |
| 21 | GGSSYDYFDY | CDRH3 ofanti-PD-L1 |
| | | antibody as disclosed in |
| | | WO 2018/205985 |
| 22 | RASESVSIHGTHLMH | CDRL1 ofanti-PD-L1 |
| | | antibody as disclosed in |
| | | WO 2018/205985 |
| 23 | AASNLES | CDRL2 ofanti-PD-L1 |
| | | antibody as disclosed in |
| | | WO 2018/205985 |
| 24 | QQSFEDPLT | CDRL3 ofanti-PD-L1 |
| | | antibody as disclosed in |
| | | WO 2018/205985 |
| 25 | QSALTQPASVSGSPGQSITISCTGTSSDVGGYNYVSWYQQHP | Bintrafusp alfa light chain |
| | GKAPKLMIYDVSNRPSGVSNRFSGSKSGNTASLTISGLQAEDE | variable region |
| | ADYYCSSYTSSSTRVFGTGTKVTVL | |
| 26 | EVQLLESGGGLVQPGGSLRLSCAASGFTFSSYIMMWVRQAP | Bintrafusp alfa heavy |
| | GKGLEWVSSIYPSGGITFYADTVKGRFTISRDNSKNTLYLQMN | chain variable region |
| | SLRAEDTAVYYCARIKLGTVTTVDYWGQGTLVTVSS | |

1-13. (canceled)

14. A kit comprising a molecule and a package insert,
wherein the molecule comprises (a) an antibody or a fragment thereof capable of binding PD-L1 and inhibiting the interaction between PD-1 and PD-L1 and (b) the extracellular domain of TGFβRII or a fragment thereof capable of binding TGFβ and inhibiting the interaction of TGFβ and the TGFβRII; and
wherein the package insert comprises instructions for using the molecule in combination with an ATM inhibitor and radiotherapy to treat or delay progression of a cancer in a subject.

15. A kit comprising an ATM inhibitor and a package insert,
wherein the package insert comprises instructions for using the ATM inhibitor in combination with a PD-1 inhibitor, a TGFβ inhibitor and radiotherapy to treat or delay progression of a cancer in a subject.

16. A method of treating cancer in a subject, the method comprising administering a PD-1 inhibitor, a TGFβ inhibitor and an ATM inhibitor to the subject in combination with radiotherapy.

17. The method of claim 16, wherein the PD-1 inhibitor is an anti-PD-L1 antibody or a fragment thereof capable of binding PD-L1.

18. The method of claim 17, wherein the anti-PD-L1 antibody or fragment thereof comprises a heavy chain sequence, which comprises a CDR1 having the sequence of SEQ ID NO: 1, a CDR2 having the sequence of SEQ ID NO: 2 and a CDR3 having the sequence of SEQ ID NO: 3, and a light chain sequence, which comprises a CDR1 having the sequence of SEQ ID NO: 4, a CDR2 having the sequence of SEQ ID NO: 5 and a CDR3 having the sequence of SEQ ID NO: 6; or
wherein the anti-PD-L1 antibody or fragment thereof comprises a heavy chain sequence, which comprises a CDR1 having the sequence of SEQ ID NO: 19, a CDR2 having the sequence of SEQ ID NO: 20 and a CDR3 having the sequence of SEQ ID NO: 21, and a light chain sequence, which comprises a CDR1 having the sequence of SEQ ID NO: 22, a CDR2 having the sequence of SEQ ID NO: 23 and a CDR3 having the sequence of SEQ ID NO: 24.

19. The method according to claim 17, wherein the anti-PD-L1 antibody or a fragment thereof capable of binding PD-L1 and the TGFβ inhibitor are fused in a molecule comprising (a) an antibody or a fragment thereof capable of binding PD-L1 and inhibiting the interaction between PD-1 and PD-L1 and (b) the extracellular domain of TGFβRII or a fragment thereof capable of binding TGFβ and inhibiting the interaction of TGFβ and the TGFβRII.

20. The method according to claim 18, wherein the anti-PD-L1 antibody or a fragment thereof capable of binding PD-L1 and the TGFβ inhibitor are fused in a molecule comprising (a) an antibody or a fragment thereof capable of binding PD-L1 and inhibiting the interaction between PD-1 and PD-L1 and (b) the extracellular domain of TGFβRII or a fragment thereof capable of binding TGFβ and inhibiting the interaction of TGFβ and the TGFβRII.

21. The method according to claim 19, wherein the extracellular domain of the TGFβRII or the fragment thereof is fused to each of the heavy chain sequences of the antibody or the fragment thereof, and wherein the light chain sequences and the sequences comprising the heavy chain sequence and the extracellular domain of TGFβRII or the fragment thereof respectively correspond to the sequences selected from the group consisting of: (1) SEQ ID NO: 7 and SEQ ID NO: 8, (2) SEQ ID NO: 15 and SEQ ID NO: 17, and (3) SEQ ID NO: 15 and SEQ ID NO: 18.

22. The method according to claim 20, wherein the amino acid sequence of the fused PD-1 inhibitor and TGFβ inhibitor is identical to the amino acid sequence of bintrafusp alfa.

23. The method according to claim 20, wherein the fused PD-1 inhibitor and TGFβ inhibitor is administered at a dose of 1200 mg once every two weeks or at a dose of 1800 mg or 2400 mg once every three weeks.

24. The method according to claim 16, wherein the ATM inhibitor is an imidazo[4,5-c]quinoline derivative.

25. The method according to claim 24, wherein the ATM inhibitor is 8-(1,3-Dimethyl-1H-pyrazol-4-yl)-1-(3-fluoro-5-methoxy-pyridin-4-yl)-7-methoxy-3-methyl-1,3-dihydro-imidazo[4,5-c]quinolin-2-one or a pharmaceutically acceptable salt thereof.

26. The method according to claim 25, wherein the ATM inhibitor is 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one or a pharmaceutically acceptable salt thereof.

27. The method according to claim 16, wherein the ATM inhibitor is administered at a dose of 25 to 400 mg per day.

28. A method for treating cancer in a subject, the method comprising administering a PD-1 inhibitor, a TGFβ inhibitor and an ATM inhibitor to the subject in combination with radiotherapy; and wherein the PD-1 inhibitor and TGFβ inhibitor are fused in a molecule having the amino acid sequence of bintrafusp alfa and the ATM inhibitor is 8-(1,3-Dimethyl-1H-pyrazol-4-yl)-1-(Sa)-(3-fluoro-5-methoxy-pyridin-4-yl)-7-methoxy-3-methyl-1,3-dihydro-imidazo[4,5-c]quinolin-2-one or a pharmaceutically acceptable salt thereof.

29. The method of claim 28, wherein the cancer is selected from the group consisting of squamous cell carcinoma, myeloma, small-cell lung cancer, non-small cell lung cancer, glioma, Hodgkin's lymphoma, non-Hodgkin's lymphoma, acute myeloid leukemia, multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma, cervical cancer, brain cancer, stomach cancer, bladder cancer, hepatoma, breast cancer, colon carcinoma, biliary tract cancer, and head and neck cancer.


SEQUENCE LISTING (26 sequences)

SEQ ID NO: 1 - protein, 5 aa, Artificial Sequence
  from human Fab library
SYIMM

SEQ ID NO: 2 - protein, 17 aa, Artificial Sequence
  from human Fab library
SIYPSGGITFYADTVKG

SEQ ID NO: 3 - protein, 11 aa, Artificial Sequence
  from human Fab library
IKLGTVTTVDY

SEQ ID NO: 4 - protein, 14 aa, Artificial Sequence
  from human Fab library
TGTSSDVGGYNYVS

SEQ ID NO: 5 - protein, 7 aa, Artificial Sequence
  from human Fab library
DVSNRPS

SEQ ID NO: 6 - protein, 10 aa, Artificial Sequence
  from human Fab library
SSYTSSSTRV

SEQ ID NO: 7 - protein, 216 aa, Artificial Sequence
  from human Fab library
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 8 - protein, 607 aa, Artificial Sequence
  Synthetic peptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 9 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 10 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 11 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 12 - protein, 117 aa, Homo sapiens
GAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 13 - protein, 115 aa, Homo sapiens
VKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 14 - protein, 446 aa, Artificial Sequence
  Description of Artificial Sequence Synthetic polypeptide
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGA

SEQ ID NO: 15 - protein, 218 aa, Artificial Sequence
  Description of Artificial Sequence Synthetic polypeptide
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 450 aa, Artificial Sequence
  Description of Artificial Sequence Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 17 - protein, 584 aa, Artificial Sequence
  Description of Artificial Sequence Synthetic polypeptide
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 18 - protein, 587 aa, Artificial Sequence
  Description of Artificial Sequence Synthetic polypeptide
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGGGGSGVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 19 - protein, 5 aa, Mus musculus
SYWMH

SEQ ID NO: 20 - protein, 17 aa, Artificial sequence
  Description of Artificial Sequence Synthetic polypeptide
  VARIANT 3: His or Gly
  VARIANT 8: Gly or Phe
RIXPNSGXTSYNEKFKN

SEQ ID NO: 21 - protein, 10 aa, Mus musculus
GGSSYDYFDY

SEQ ID NO: 22 - protein, 15 aa, Mus musculus
RASESVSIHGTHLMH

SEQ ID NO: 23 - protein, 7 aa, Mus musculus
AASNLES

SEQ ID NO: 24 - protein, 9 aa, Mus musculus
QQSFEDPLT

SEQ ID NO: 25 - protein, 110 aa, Artificial Sequence
  From human Fab library
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 26 - protein, 120 aa, Artificial Sequence
  From human Fab library
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS